(19) United States
(12) Patent Application Publication
(10) Pub. No.: US 2025/0041434 A1
(43) Pub. Date: Feb. 6, 2025

(54) CEA ASSAY FOR PATIENT SELECTION IN CANCER THERAPY

(71) Applicant: SANOFI, Paris (FR)

(72) Inventors: Mustapha CHADJAA, Paris (FR); Anne-Laure BAUCHET, Paris (FR); Céline COMBEAU, Paris (FR); Christophe HENRY, Paris (FR); Brigitte DEMERS, Paris (FR); Samira BENSFIA, Cambridge, MA (US)

(73) Assignee: SANOFI, Paris, FR (FR)

(21) Appl. No.: 18/715,666

(22) PCT Filed: Dec. 1, 2022
(86) PCT No.: PCT/EP2022/084107

§ 371 Date: May 31, 2024

Related U.S. Application Data
(60) Provisional application No. 63/385,375, filed on Nov. 29, 2022.

(30) Foreign Application Priority Data
Dec. 2, 2021 (EP) 21306692.1

(51) Int. Cl. A61K 47/68; A61K 31/519; A61K 31/555; A61K 33/243; A61K 45/06; A61P 35/00; C07K 16/28; C07K 16/30; G01N 33/574; G01N 33/68

(52) U.S. Cl. CPC A61K 47/68033; A61K 31/519; A61K 31/555; A61K 33/243; A61K 45/06; A61K 47/6853; A61P 35/00; C07K 16/2818; C07K 16/3007; G01N 33/57423; G01N 33/57446; G01N 33/6893; G01N 2474/20

(57) ABSTRACT

Provided are methods for identifying and treating patients with cancer, where the cancer expresses CEACAM5. The methods include measuring circulating CEA to identify patients likely to benefit from treatment with an agent specific for CEACAM5. Such patients can have high circulating CEA and only low or medium expression of CEACAM5 on tumor cells as measured by immunohistochemistry (IHC). The agent specific for CEACAM5 can be tusamitamab ravtansine. Such agent can be used in combination with one or more additional agents to treat the cancer. In certain embodiments the cancer is non-squamous non-small cell lung cancer (NSQ NSCLC).

### REFERENCE TO SEQUENCE LISTING

The instant application contains a Sequence Listing which has been submitted electronically in .xml format and is hereby incorporated by reference in its entirety. Said .xml copy, created on Nov. 28, 2022, is named PR94291_S287_WO_SANOFI_SEQUENCES.xml.

### FIELD

The present disclosure relates to the field of cancer therapy.

### BACKGROUND

Despite recent advances in the treatment of cancer, there remains a need for effective new treatment at the time of disease progression after first-line therapy. Current therapeutic approaches for subsequent systemic options combining an inhibitor of angiogenesis with a systemic cytotoxic agent such as docetaxel entail serious hematological and other toxicities. Docetaxel, pemetrexed, or gemcitabine used as single cytotoxic agents offer very limited other options. Therefore, targeted cytotoxic therapies may offer an improvement in safety and tolerability as well as efficacy.

One feature that can be used to target some tumor cells is surface expression of carcinoembryonic antigen-related cell adhesion molecule 5 (CEACAM5), first described in 1965 as a tumor-associated antigen in human colon cancer tissue extracts (Gold P et al., J Exp Med. 1965; 122(3):467-481). High levels of CEACAM5 expression have since been observed in several epithelial tumors, whereas in normal adult tissue, its expression is limited to few tissues (Hammarstrom S., Semin Cancer Biol. 1999; 9(2):67-81; Thompson J A., Tumor Biol. 1995; 16(1):10-16).

Tusamitamab ravtansine is an antibody to CEACAM5 conjugated to the cytotoxic maytansinoid agent N2′-deacetyl-N-2′(4-methyl-4-mercapto-1-oxopentyl)-maytansine (DM4). Encouraging preliminary anti-tumor activity of tusamitamab ravtansine in participants heavily pre-treated for NSQ NSCLC has been demonstrated in an ongoing study (TED13751).

### SUMMARY

The disclosure is based at least in based at least in part on the observation that particular levels of circulating carcinoembryonic antigen (CEA) are useful as a biomarker for selection of patients for treatment of cancers which typically express CEA Cell Adhesion Molecule 5 (CEACAM5) on their cells.

An aspect of the disclosure is a method of treating a cancer in a subject, comprising
measuring circulating carcinoembryonic antigen (CEA) in a subject in need of treatment of cancer; and
administering to the subject an effective amount of an antibody-drug conjugate (ADC) comprising an anti-CEACAM5 antibody if the circulating CEA in the subject is ≥5 ng/mL (≥5 μg/L),

thereby treating the cancer.

An aspect of the disclosure is a circulating carcinoembryonic antigen (CEA) for use as a biomarker in methods as disclosed herein.

In some embodiments, a circulating carcinoembryonic antigen (CEA) may be for use as a biomarker in a method for treating a cancer in a subject, as disclosed herein.

In some embodiments, a circulating carcinoembryonic antigen (CEA) may be for use as a biomarker in a method for diagnosing a cancer in a subject, as disclosed herein.

An aspect of the disclosure is a circulating carcinoembryonic antigen (CEA) for use as a biomarker in a method for treating a cancer in a subject, the method comprising

measuring circulating carcinoembryonic antigen (CEA) in a subject in need of treatment of cancer; and

administering to the subject an effective amount of an antibody-drug conjugate (ADC) comprising an anti-CEACAM5 antibody if the circulating CEA in the subject is about ≥5 ng/mL,

thereby treating the cancer.

In a use as disclosed herein, the biomarker is measured in an isolated biological sample.

In some embodiments, the disclosure relates to an antibody-drug conjugate (ADC) comprising an anti-CEACAM5 antibody conjugated to a cytotoxic agent, for use in a method of treating a cancer in a subject in need thereof,

the anti-CEACAM5 antibody comprising a HCDR1 having the amino acid sequence of SEQ ID NO: 1, a HCDR2 having the amino acid sequence of SEQ ID NO: 2, a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5,

the cytotoxic agent being maytansinoid or maytansinoid analog, and

the method comprising measuring circulating carcinoembryonic antigen (CEA) in said subject and administering to said subject an effective amount of said ADC if the circulating CEA in said subject is about ≥5 ng/mL, thereby treating the cancer.

In some embodiments, the disclosure relates to a use of an antibody-drug conjugate (ADC) comprising an anti-CEACAM5 antibody conjugated to a cytotoxic agent, for manufacturing a medicament for use in a method of treating a cancer in a subject in need thereof,

the anti-CEACAM5 antibody comprising a HCDR1 having the amino acid sequence of SEQ ID NO: 1, a HCDR2 having the amino acid sequence of SEQ ID NO: 2, a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5,

the cytotoxic agent being maytansinoid or maytansinoid analog, and

the method comprising measuring circulating carcinoembryonic antigen (CEA) in said subject and administering to said subject an effective amount of said medicament if the circulating CEA in said subject is about ≥5 ng/mL, thereby treating the cancer.

“Biomarker” intends to refer a biological molecule, for example a protein or a metabolite, that is differentially present, increased or decreased, in a biological sample obtained from a subject or a group of subjects having a first phenotype, e.g., having a disease such as a cancer, as compared to a biological sample from a subject or a group of subjects having a second phenotype, e.g., not having the disease. In use, the biomarker is isolated from the subject.

“Sample” or “biological sample” intends to refer to a biological material isolated from a subject. The biological sample may contain any biological material suitable for detecting the biomarker, i.e., circulating carcinoembryonic antigen (CEA), and may comprise cellular and/or non-cellular material from the subject. The sample may be isolated from any suitable biological tissue or fluid such as, for example, kidney tissue, blood, blood plasma (plasma), blood serum (serum), urine, or cerebral spinal fluid (CSF). In some embodiments, a biological sample is a blood plasma (plasma) or a blood serum (serum) sample,

In a use as disclosed herein, the measured circulating carcinoembryonic antigen (CEA) in a subject in need of treatment of cancer may be compared to a value of reference. A value of reference may be 5 ng/mL A measured circulating carcinoembryonic antigen (CEA) in a subject about ≥5 ng/mL may be indicative of a cancer.

A “value of reference” intends to refer to a level of the circulating CEA that is indicative of a particular disease state, phenotype, such as cancer, or lack thereof, as well as combinations of disease states, phenotypes, or lack thereof, in the concerned subject.

An aspect of the disclosure is a method of diagnosing a cancer in a subject, the method comprising at least the steps of:
a) measuring in a biological sample an amount of circulating carcinoembryonic antigen (CEA) in said subject; and
b) comparing the measured amount obtained at step a) with a value of reference, said value of reference being 5 ng/mL,

a measured amount obtained at step a) about ≥5 ng/mL being indicative of a cancer.

In certain embodiments, the anti-CEACAM5 antibody is tusamitamab.

In certain embodiments, the ADC is tusamitamab ravtansine.

In certain embodiments, the cancer is selected from the group consisting of colorectal cancer, gastric cancer, gastroesophageal junction cancer, esophageal cancer, lung cancer, uterine cervix cancer, pancreatic cancer, ovarian cancer, thyroid cancer, bladder cancer, endometrial cancer, breast cancer, liver cancer, biliary tract cancer (e.g., cholangiocarcinoma), prostate cancer, and skin cancer.

In certain embodiments, the cancer is gastric cancer, gastroesophageal junction cancer, or esophageal cancer.

In certain embodiments, the cancer is gastric cancer.

In certain embodiments, the cancer is lung cancer.

In certain embodiments, the cancer is non-squamous non-small cell lung cancer (NSQ NSCLC).

In certain embodiments, the cancer is advanced or metastatic.

In certain embodiments, the NSQ NSCLC is advanced or metastatic.

In certain embodiments, the circulating CEA is measured after performing immunohistochemistry analysis of CEACAM5 expression on tumor cells of the subject.

In certain embodiments, the subject has negative or low CEACAM5 expression on tumor cells (≥2+ intensity in <1% of cells) as measured by immunohistochemistry.

In certain embodiments, the subject has moderate CEACAM5 expression on tumor cells (≥2+ intensity in ≥1% and <50% of cells) as measured by immunohistochemistry.

In certain embodiments, the subject has high CEACAM5 expression on tumor cells (≥2+ intensity in ≥50% of cells) as measured by immunohistochemistry.

In certain embodiments, prior to treatment the circulating CEA is about ≥20 ng/mL.

In certain embodiments, prior to treatment the circulating CEA is about ≥25 ng/mL.

In certain embodiments, prior to treatment the circulating CEA is about ≥30 ng/mL.

In certain embodiments, prior to treatment the circulating CEA is about ≥35 ng/mL.

In certain embodiments, prior to treatment the circulating CEA is about ≥40 ng/mL.

In certain embodiments, prior to treatment the circulating CEA is about ≥45 ng/mL.

In certain embodiments, prior to treatment the circulating CEA is about ≥50 ng/mL.

In certain embodiments, prior to treatment the circulating CEA is about ≥55 ng/mL.

In certain embodiments, prior to treatment the circulating CEA is about ≥60 ng/mL.

In certain embodiments, prior to treatment the circulating CEA is about ≥61 ng/mL.

In certain embodiments, prior to treatment the circulating CEA is about ≥62 ng/mL.

In certain embodiments, prior to treatment the circulating CEA is about ≥63 ng/mL.

In certain embodiments, prior to treatment the circulating CEA is about ≥64 ng/mL.

In certain embodiments, prior to treatment the circulating CEA is about ≥65 ng/mL.

In certain embodiments, prior to treatment the circulating CEA is about ≥66 ng/mL.

In certain embodiments, prior to treatment the circulating CEA is about ≥67 ng/mL.

In certain embodiments, prior to treatment the circulating CEA is about ≥68 ng/mL.

In certain embodiments, prior to treatment the circulating CEA is about ≥69 ng/mL.

In certain embodiments, prior to treatment the circulating CEA is about ≥70 ng/mL.

In certain embodiments, prior to treatment the circulating CEA is about ≥71 ng/mL.

In certain embodiments, prior to treatment the circulating CEA is about ≥72 ng/mL.

In certain embodiments, prior to treatment the circulating CEA is about ≥73 ng/mL.

In certain embodiments, prior to treatment the circulating CEA is about ≥74 ng/mL.

In certain embodiments, prior to treatment the circulating CEA is about ≥75 ng/mL.

In certain embodiments, prior to treatment the circulating CEA is about ≥76 ng/mL.

In certain embodiments, prior to treatment the circulating CEA is about ≥77 ng/mL.

In certain embodiments, prior to treatment the circulating CEA is about ≥78 ng/mL.

In certain embodiments, prior to treatment the circulating CEA is about ≥79 ng/mL.

In certain embodiments, prior to treatment the circulating CEA is about ≥80 ng/mL

In certain embodiments, prior to treatment the circulating CEA is about ≥85 ng/mL.

In certain embodiments, prior to treatment the circulating CEA is about ≥90 ng/mL.

In certain embodiments, prior to treatment the circulating CEA is about ≥95 ng/mL.

In certain embodiments, prior to treatment the circulating CEA is about ≥100 ng/mL.

In certain embodiments, the tusamitamab ravtansine is administered in a dose of about 60 mg/m² to about 190 mg/m² (e.g., about once every two or three weeks).

In certain embodiments, the tusamitamab ravtansine is administered about once every two weeks. In certain embodiments, the tusamitamab ravtansine is administered about once every three weeks. In certain embodiments, the tusamitamab ravtansine is administered about once every four weeks. In certain embodiments, the tusamitamab ravtansine is administered about once every five weeks. In certain embodiments, the tusamitamab ravtansine is administered about once every six weeks.

In certain embodiments, the tusamitamab ravtansine is administered in a dose of about 60 mg/m² (e.g., about once every two or three weeks).

In certain embodiments, the tusamitamab ravtansine is administered in a dose of about 100 mg/m² (e.g., about once every two or three weeks).

In certain embodiments, the tusamitamab ravtansine is administered in a dose of about 120 mg/m² (e.g., about once every two or three weeks).

In certain embodiments, the tusamitamab ravtansine is administered in a dose of about 150 mg/m² (e.g., about once every two or three weeks).

In certain embodiments, the tusamitamab ravtansine is administered in a dose of about 170 mg/m² (e.g., about once every two or three weeks).

In certain embodiments, the tusamitamab ravtansine is administered in a dose of about ≥100 mg/m² (e.g., 100 mg/m²) about once every two weeks.

In certain embodiments, the tusamitamab ravtansine is administered in a dose of about ≥100 mg/m² (e.g., 100 mg/m²) about once every three weeks.

In certain embodiments, the tusamitamab ravtansine is administered in a dose of about 100 mg/m² about once every two weeks.

In certain embodiments, the tusamitamab ravtansine is administered in a dose of about 100 mg/m² about once every three weeks.

In certain embodiments, the method further comprises administering to the subject an effective amount of at least one additional agent effective to treat the cancer.

In certain embodiments, the additional agent is selected from the group consisting of an immune checkpoint inhibitor (ICI), a platinum-based chemotherapy (e.g., cisplatin or carboplatin), pemetrexed, anti-VEGFR2, FOLFOX, FOLFIRI, TAS-102, anti-EGFR, and any combination thereof.

In certain embodiments, the ICI is an anti-PD-1 antibody.

In certain embodiments, the anti-PD-1 antibody is selected from the group consisting of pembrolizumab, nivolumab, cemiplimab, sintilimab, dostarlimab, and tislelizumab.

In certain embodiments, the anti-PD-1 antibody is pembrolizumab.

In certain embodiments, the ICI is an anti-PD-L1 antibody.

In certain embodiments, the anti-PD-L1 antibody is selected from the group consisting of atezolizumab, avelumab, durvalumab, envafolimab, BMS-936559, CK-301, CS-1001, SHR-1316 (HTI-1088), CBT-502 (TQB-2450), and any combination thereof.

In certain embodiments, the anti-PD-L1 antibody is selected from the group consisting of atezolizumab, avelumab, and durvalumab.

In certain embodiments, the method comprises administering to the subject an effective amount of tusamitamab ravtansine and pembrolizumab.

In certain embodiments, the method further comprises administering to the subject an effective amount of a platinum-based chemotherapy.

In certain embodiments, the platinum-based chemotherapy is selected from cisplatin and carboplatin.

In certain embodiments, the method further comprises administering to the subject an effective amount of pemetrexed.

In certain embodiments, the method comprises administering to the subject an effective amount of tusamitamab ravtansine, pembrolizumab, and cisplatin.

In certain embodiments, the method comprises administering to the subject an effective amount of tusamitamab ravtansine, pembrolizumab, cisplatin, and pemetrexed.

In certain embodiments, the method comprises administering to the subject an effective amount of tusamitamab ravtansine, pembrolizumab, and carboplatin.

In certain embodiments, the method comprises administering to the subject an effective amount of tusamitamab ravtansine, pembrolizumab, carboplatin, and pemetrexed.

Another aspect of the disclosure is a method of treating a cancer in a subject, comprising
selecting a subject having cancer with a circulating carcinoembryonic antigen (CEA) about ≥5 ng/mL; and
administering to the subject an effective amount of an antibody-drug conjugate (ADC) comprising an anti-CEACAM5 antibody,

thereby treating the cancer.

Another aspect of the disclosure is a circulating carcinoembryonic antigen (CEA) for use as a biomarker in a method of treating a cancer in a subject, the method comprising

selecting a subject having cancer with a circulating carcinoembryonic antigen (CEA) about ≥5 ng/mL; and

administering to the subject an effective amount of an antibody-drug conjugate (ADC) comprising an anti-CEACAM5 antibody,

thereby treating the cancer.

In certain embodiments, the anti-CEACAM5 antibody is tusamitamab.

In certain embodiments, the ADC is tusamitamab ravtansine.

In certain embodiments, the cancer is selected from the group consisting of colorectal cancer, gastric cancer, gastroesophageal junction cancer, esophageal cancer, lung cancer, uterine cervix cancer, pancreatic cancer, ovarian cancer, thyroid cancer, bladder cancer, endometrial cancer, breast cancer, liver cancer, biliary tract cancer (e.g., cholangiocarcinoma), prostate cancer, and skin cancer.

In certain embodiments, the cancer is gastric cancer, gastroesophageal junction cancer, or esophageal cancer.

In certain embodiments, the cancer is gastric cancer.

In certain embodiments, the cancer is lung cancer.

In certain embodiments, the cancer is non-squamous non-small cell lung cancer (NSQ NSCLC).

In certain embodiments, the cancer is advanced or metastatic.

In certain embodiments, the NSQ NSCLC is advanced or metastatic.

In certain embodiments, the circulating CEA is measured after performing immunohistochemistry analysis of CEACAM5 expression on tumor cells of the subject.

In certain embodiments, the subject has negative or low CEACAM5 expression on tumor cells (≥2+ intensity in <1% of cells) as measured by immunohistochemistry.

In certain embodiments, the subject has moderate CEACAM5 expression on tumor cells (≥2+ intensity in ≥1% and <50% of cells) as measured by immunohistochemistry.

In certain embodiments, the subject has high CEACAM5 expression on tumor cells (≥2+ intensity in ≥50% of cells) as measured by immunohistochemistry.

In certain embodiments, prior to treatment the circulating CEA is about ≥20 ng/mL.

In certain embodiments, prior to treatment the circulating CEA is about ≥25 ng/mL.

In certain embodiments, prior to treatment the circulating CEA is about ≥30 ng/mL.

In certain embodiments, prior to treatment the circulating CEA is about ≥35 ng/mL.

In certain embodiments, prior to treatment the circulating CEA is about ≥40 ng/mL.

In certain embodiments, prior to treatment the circulating CEA is about ≥45 ng/mL.

In certain embodiments, prior to treatment the circulating CEA is about ≥50 ng/mL.

In certain embodiments, prior to treatment the circulating CEA is about ≥55 ng/mL.

In certain embodiments, prior to treatment the circulating CEA is about ≥60 ng/mL.

In certain embodiments, prior to treatment the circulating CEA is about ≥61 ng/mL.

In certain embodiments, prior to treatment the circulating CEA is about ≥62 ng/mL.

In certain embodiments, prior to treatment the circulating CEA is about ≥63 ng/mL.

In certain embodiments, prior to treatment the circulating CEA is about ≥64 ng/mL.

In certain embodiments, prior to treatment the circulating CEA is about ≥65 ng/mL.

In certain embodiments, prior to treatment the circulating CEA is about ≥66 ng/mL.

In certain embodiments, prior to treatment the circulating CEA is about ≥67 ng/mL.

In certain embodiments, prior to treatment the circulating CEA is about ≥68 ng/mL.

In certain embodiments, prior to treatment the circulating CEA is about ≥69 ng/mL.

In certain embodiments, prior to treatment the circulating CEA is about ≥70 ng/mL.

In certain embodiments, prior to treatment the circulating CEA is about ≥71 ng/mL.

In certain embodiments, prior to treatment the circulating CEA is about ≥72 ng/mL.

In certain embodiments, prior to treatment the circulating CEA is about ≥73 ng/mL.

In certain embodiments, prior to treatment the circulating CEA is about ≥74 ng/mL.

In certain embodiments, prior to treatment the circulating CEA is about ≥75 ng/mL.

In certain embodiments, prior to treatment the circulating CEA is about ≥76 ng/mL.

In certain embodiments, prior to treatment the circulating CEA is about ≥77 ng/mL.

In certain embodiments, prior to treatment the circulating CEA is about ≥78 ng/mL.

In certain embodiments, prior to treatment the circulating CEA is about ≥79 ng/mL.

In certain embodiments, prior to treatment the circulating CEA is about ≥80 ng/mL

In certain embodiments, prior to treatment the circulating CEA is about ≥85 ng/mL.

In certain embodiments, prior to treatment the circulating CEA is about ≥90 ng/mL.

In certain embodiments, prior to treatment the circulating CEA is about ≥95 ng/mL.

In certain embodiments, prior to treatment the circulating CEA is about ≥100 ng/mL.

In certain embodiments, the tusamitamab ravtansine is administered in a dose of about 60 mg/m² to about 190 mg/m² (e.g., about once every two or three weeks).

In certain embodiments, the tusamitamab ravtansine is administered about once every two weeks. In certain embodiments, the tusamitamab ravtansine is administered about once every three weeks. In certain embodiments, the tusamitamab ravtansine is administered about once every four weeks. In certain embodiments, the tusamitamab ravtansine is administered about once every five weeks. In certain embodiments, the tusamitamab ravtansine is administered about once every six weeks.

In certain embodiments, the tusamitamab ravtansine is administered in a dose of about 60 mg/m² (e.g., about once every two or three weeks).

In certain embodiments, the tusamitamab ravtansine is administered in a dose of about 100 mg/m² (e.g., about once every two or three weeks).

In certain embodiments, the tusamitamab ravtansine is administered in a dose of about 120 mg/m² (e.g., about once every two or three weeks).

In certain embodiments, the tusamitamab ravtansine is administered in a dose of about 150 mg/m² (e.g., about once every two or three weeks).

In certain embodiments, the tusamitamab ravtansine is administered in a dose of about 170 mg/m² (e.g., about once every two or three weeks).

In certain embodiments, the tusamitamab ravtansine is administered in a dose of about ≥100 mg/m² (e.g., 100 mg/m²) about once every two weeks.

In certain embodiments, the tusamitamab ravtansine is administered in a dose of about ≥100 mg/m² (e.g., 100 mg/m²) about once every three weeks.

In certain embodiments, the tusamitamab ravtansine is administered in a dose of about 100 mg/m² about once every two weeks.

In certain embodiments, the tusamitamab ravtansine is administered in a dose of about 100 mg/m² about once every three weeks.

In certain embodiments, the method further comprises administering to the subject an effective amount of at least one additional agent effective to treat the cancer.

In certain embodiments, the additional agent is selected from the group consisting of an immune checkpoint inhibitor (ICI), a platinum-based chemotherapy (e.g., cisplatin or carboplatin), pemetrexed, anti-VEGFR2, FOLFOX, FOLFIRI, TAS-102, anti-EGFR, and any combination thereof.

In certain embodiments, the ICI is an anti-PD-1 antibody.

In certain embodiments, the anti-PD-1 antibody is selected from the group consisting of pembrolizumab, nivolumab, cemiplimab, sintilimab, dostarlimab, and tislelizumab.

In certain embodiments, the anti-PD-1 antibody is pembrolizumab.

In certain embodiments, the ICI is an anti-PD-L1 antibody.

In certain embodiments, the anti-PD-L1 antibody is selected from the group consisting of atezolizumab, avelumab, durvalumab, envafolimab, BMS-936559, CK-301, CS-1001, SHR-1316 (HTI-1088), CBT-502 (TQB-2450), and any combination thereof.

In certain embodiments, the anti-PD-L1 antibody is selected from the group consisting of atezolizumab, avelumab, and durvalumab.

In certain embodiments, the method comprises administering to the subject an effective amount of tusamitamab ravtansine and pembrolizumab.

In certain embodiments, the method further comprises administering to the subject an effective amount of a platinum-based chemotherapy.

In certain embodiments, the platinum-based chemotherapy is selected from cisplatin and carboplatin.

In certain embodiments, the method further comprises administering to the subject an effective amount of pemetrexed.

In certain embodiments, the method comprises administering to the subject an effective amount of tusamitamab ravtansine, pembrolizumab, and cisplatin.

In certain embodiments, the method comprises administering to the subject an effective amount of tusamitamab ravtansine, pembrolizumab, cisplatin, and pemetrexed.

In certain embodiments, the method comprises administering to the subject an effective amount of tusamitamab ravtansine, pembrolizumab, and carboplatin.

In certain embodiments, the method comprises administering to the subject an effective amount of tusamitamab ravtansine, pembrolizumab, carboplatin, and pemetrexed.

Another aspect of the disclosure is a method of treating a cancer in a subject, comprising
measuring a first circulating carcinoembryonic antigen (CEA) in a subject in need of treatment of cancer;
administering to the subject a first effective amount of an antibody-drug conjugate (ADC) comprising an anti-CEACAM5 antibody if the first measured circulating CEA in the subject prior to the administering is about ≥5 ng/mL;
measuring a second circulating CEA in the subject after the administering step; and
administering to the subject a second effective amount of the ADC if the second measured circulating CEA in the subject is less than the first measured circulating CEA,

thereby treating the cancer.

Another aspect of the disclosure is a circulating carcinoembryonic antigen (CEA) for use as a biomarker in a method of treating a cancer in a subject, the method comprising

measuring a first circulating carcinoembryonic antigen (CEA) in a subject in need of treatment of cancer;

administering to the subject a first effective amount of an antibody-drug conjugate (ADC) comprising an anti-CEACAM5 antibody if the first measured circulating CEA in the subject prior to the administering is about ≥5 ng/mL;

measuring a second circulating CEA in the subject after the administering step; and

administering to the subject a second effective amount of the ADC if the second measured circulating CEA in the subject is less than the first measured circulating CEA,

thereby treating the cancer.

In certain embodiments, the anti-CEACAM5 antibody is tusamitamab.

In certain embodiments, the ADC is tusamitamab ravtansine.

In certain embodiments, the cancer is selected from the group consisting of colorectal cancer, gastric cancer, gastroesophageal junction cancer, esophageal cancer, lung cancer, uterine cervix cancer, pancreatic cancer, ovarian cancer, thyroid cancer, bladder cancer, endometrial cancer, breast cancer, liver cancer, biliary tract cancer (e.g., cholangiocarcinoma), prostate cancer, and skin cancer.

In certain embodiments, the cancer is gastric cancer.

In certain embodiments, the cancer is lung cancer.

In certain embodiments, the cancer is non-squamous non-small cell lung cancer (NSQ NSCLC).

In certain embodiments, the cancer is advanced or metastatic.

In certain embodiments, the NSQ NSCLC is advanced or metastatic.

In certain embodiments, the circulating CEA is measured after performing immunohistochemistry analysis of CEACAM5 expression on tumor cells of the subject.

In certain embodiments, the subject has negative or low CEACAM5 expression on tumor cells (≥2+ intensity in <1% of cells) as measured by immunohistochemistry.

In certain embodiments, the subject has moderate CEACAM5 expression on tumor cells (≥2+ intensity in ≥1% and <50% of cells) as measured by immunohistochemistry.

In certain embodiments, the subject has high CEACAM5 expression on tumor cells (≥2+ intensity in ≥50% of cells) as measured by immunohistochemistry.

In certain embodiments, prior to treatment the circulating CEA is about ≥20 ng/mL.

In certain embodiments, prior to treatment the circulating CEA is about ≥25 ng/mL.

In certain embodiments, prior to treatment the circulating CEA is about ≥30 ng/mL.

In certain embodiments, prior to treatment the circulating CEA is about ≥35 ng/mL.

In certain embodiments, prior to treatment the circulating CEA is about ≥40 ng/mL.

In certain embodiments, prior to treatment the circulating CEA is about ≥45 ng/mL.

In certain embodiments, prior to treatment the circulating CEA is about ≥50 ng/mL.

In certain embodiments, prior to treatment the circulating CEA is about ≥55 ng/mL.

In certain embodiments, prior to treatment the circulating CEA is about ≥60 ng/mL.

In certain embodiments, prior to treatment the circulating CEA is about ≥61 ng/mL.

In certain embodiments, prior to treatment the circulating CEA is about ≥62 ng/mL.

In certain embodiments, prior to treatment the circulating CEA is about ≥63 ng/mL.

In certain embodiments, prior to treatment the circulating CEA is about ≥64 ng/mL.

In certain embodiments, prior to treatment the circulating CEA is about ≥65 ng/mL.

In certain embodiments, prior to treatment the circulating CEA is about ≥66 ng/mL.

In certain embodiments, prior to treatment the circulating CEA is about ≥67 ng/mL.

In certain embodiments, prior to treatment the circulating CEA is about ≥68 ng/mL.

In certain embodiments, prior to treatment the circulating CEA is about ≥69 ng/mL.

In certain embodiments, prior to treatment the circulating CEA is about ≥70 ng/mL.

In certain embodiments, prior to treatment the circulating CEA is about ≥71 ng/mL.

In certain embodiments, prior to treatment the circulating CEA is about ≥72 ng/mL.

In certain embodiments, prior to treatment the circulating CEA is about ≥73 ng/mL.

In certain embodiments, prior to treatment the circulating CEA is about ≥74 ng/mL.

In certain embodiments, prior to treatment the circulating CEA is about ≥75 ng/mL.

In certain embodiments, prior to treatment the circulating CEA is about ≥76 ng/mL.

In certain embodiments, prior to treatment the circulating CEA is about ≥77 ng/mL.

In certain embodiments, prior to treatment the circulating CEA is about ≥78 ng/mL.

In certain embodiments, prior to treatment the circulating CEA is about ≥79 ng/mL.

In certain embodiments, prior to treatment the circulating CEA is about ≥80 ng/mL

In certain embodiments, prior to treatment the circulating CEA is about ≥85 ng/mL.

In certain embodiments, prior to treatment the circulating CEA is about ≥90 ng/mL.

In certain embodiments, prior to treatment the circulating CEA is about ≥95 ng/mL.

In certain embodiments, prior to treatment the circulating CEA is about ≥100 ng/mL.

In certain embodiments, the tusamitamab ravtansine is administered in a dose of about about 60 mg/m² to about 190 mg/m² (e.g., about once every two or three weeks).

In certain embodiments, the tusamitamab ravtansine is administered about once every two weeks. In certain embodiments, the tusamitamab ravtansine is administered about once every three weeks. In certain embodiments, the tusamitamab ravtansine is administered about once every four weeks. In certain embodiments, the tusamitamab ravtansine is administered about once every five weeks. In certain embodiments, the tusamitamab ravtansine is administered about once every six weeks.

In certain embodiments, the tusamitamab ravtansine is administered in a dose of about 60 mg/m² (e.g., about once every two or three weeks).

In certain embodiments, the tusamitamab ravtansine is administered in a dose of about 100 mg/m² (e.g., about once every two or three weeks).

In certain embodiments, the tusamitamab ravtansine is administered in a dose of about 120 mg/m² (e.g., about once every two or three weeks).

In certain embodiments, the tusamitamab ravtansine is administered in a dose of about 150 mg/m² (e.g., about once every two or three weeks).

In certain embodiments, the tusamitamab ravtansine is administered in a dose of about 170 mg/m² (e.g., about once every two or three weeks).

In certain embodiments, the tusamitamab ravtansine is administered in a dose of about ≥100 mg/m² (e.g., 100 mg/m²) about once every two weeks.

In certain embodiments, the tusamitamab ravtansine is administered in a dose of about ≥100 mg/m² (e.g., 100 mg/m²) about once every three weeks.

In certain embodiments, the tusamitamab ravtansine is administered in a dose of about 100 mg/m² about once every two weeks.

In certain embodiments, the tusamitamab ravtansine is administered in a dose of about 100 mg/m² about once every three weeks.

In certain embodiments, the method further comprises administering to the subject an effective amount of at least one additional agent effective to treat the cancer.

In certain embodiments, the additional agent is selected from the group consisting of an immune checkpoint inhibitor (ICI), a platinum-based chemotherapy (e.g., cisplatin or carboplatin, pemetrexed, anti-VEGFR2, FOLFOX, FOLFIRI, TAS-102, anti-EGFR, and any combination thereof.

In certain embodiments, the ICI is an anti-PD-1 antibody.

In certain embodiments, the anti-PD-1 antibody is selected from the group consisting of pembrolizumab, nivolumab, cemiplimab, sintilimab, dostarlimab, and tislelizumab.

In certain embodiments, the anti-PD-1 antibody is pembrolizumab.

In certain embodiments, the ICI is an anti-PD-L1 antibody.

In certain embodiments, the anti-PD-L1 antibody is selected from the group consisting of atezolizumab, avelumab, durvalumab, envafolimab, BMS-936559, CK-301, CS-1001, SHR-1316 (HTI-1088), CBT-502 (TQB-2450), and any combination thereof.

In certain embodiments, the anti-PD-L1 antibody is selected from the group consisting of atezolizumab, avelumab, and durvalumab.

In certain embodiments, the method comprises administering to the subject an effective amount of tusamitamab ravtansine and pembrolizumab.

In certain embodiments, the method further comprises administering to the subject an effective amount of a platinum-based chemotherapy.

In certain embodiments, the platinum-based chemotherapy is selected from cisplatin and carboplatin.

In certain embodiments, the method further comprises administering to the subject an effective amount of pemetrexed.

In certain embodiments, the method comprises administering to the subject an effective amount of tusamitamab ravtansine, pembrolizumab, and cisplatin.

In certain embodiments, the method comprises administering to the subject an effective amount of tusamitamab ravtansine, pembrolizumab, cisplatin, and pemetrexed.

In certain embodiments, the method comprises administering to the subject an effective amount of tusamitamab ravtansine, pembrolizumab, and carboplatin.

In certain embodiments, the method comprises administering to the subject an effective amount of tusamitamab ravtansine, pembrolizumab, carboplatin, and pemetrexed.

In an embodiment 1, the present disclosure relates to a circulating carcinoembryonic antigen (CEA) for use as a biomarker in a method of treating a cancer in a subject, the method comprising

measuring circulating carcinoembryonic antigen (CEA) in a subject in need of treatment of cancer; and

administering to the subject an effective amount of an antibody-drug conjugate (ADC) comprising an anti-CEACAM5 antibody if the circulating CEA in the subject is about ≥5 ng/mL,

thereby treating the cancer.

In an embodiment 2, the present disclosure relates to a circulating carcinoembryonic antigen (CEA) for use as a biomarker in a method of treating a cancer in a subject, the method comprising

selecting a subject having cancer with a circulating carcinoembryonic antigen (CEA) about ≥5 ng/mL; and

administering to the subject an effective amount of an antibody-drug conjugate (ADC) comprising an anti-CEACAM5 antibody,

thereby treating the cancer.

In an embodiment 3, the present disclosure relates to a circulating carcinoembryonic antigen (CEA) for use as a biomarker in a method of treating a cancer in a subject, the method comprising

measuring a first circulating carcinoembryonic antigen (CEA) in a subject in need of treatment of cancer;

administering to the subject a first effective amount of an antibody-drug conjugate (ADC) comprising an anti-CEACAM5 antibody if the first measured circulating CEA in the subject prior to the administering is about ≥5 ng/mL;

measuring a second circulating CEA in the subject after the administering step; and

administering to the subject a second effective amount of the ADC if the second measured circulating CEA in the subject is less than the first measured circulating CEA, thereby treating the cancer.

In embodiment 4, the disclosure relates to an antibody-drug conjugate (ADC) comprising an anti-CEACAM5 antibody conjugated to a cytotoxic agent, for use in a method of treating a cancer in a subject in need thereof,

the anti-CEACAM5 antibody comprising a HCDR1 having the amino acid sequence of SEQ ID NO: 1, a HCDR2 having the amino acid sequence of SEQ ID NO: 2, a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5,

the cytotoxic agent being maytansinoid or maytansinoid analog, and

the method comprising measuring circulating carcinoembryonic antigen (CEA) in said subject and administering to said subject an effective amount of said ADC if the circulating CEA in said subject is about ≥5 ng/mL, thereby treating the cancer.

In embodiment 5, the disclosure relates to a use of an antibody-drug conjugate (ADC) comprising an anti-CEACAM5 antibody conjugated to a cytotoxic agent, for manufacturing a medicament for use in a method of treating a cancer in a subject in need thereof,

the anti-CEACAM5 antibody comprising a HCDR1 having the amino acid sequence of SEQ ID NO: 1, a HCDR2 having the amino acid sequence of SEQ ID NO: 2, a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5,

the cytotoxic agent being maytansinoid or maytansinoid analog, and

the method comprising measuring circulating carcinoembryonic antigen (CEA) in said subject and administering to said subject an effective amount of said medicament if the circulating CEA in said subject is about ≥5 ng/mL, thereby treating the cancer.

In embodiment 6, the disclosure relates to a method of treating a cancer in a subject in need thereof, the method comprising the steps of:

measuring circulating carcinoembryonic antigen (CEA) in said subject, and

administering to said subject an effective amount of an antibody-drug conjugate ADC if the circulating CEA in said subject is about ≥5 ng/mL, thereby treating the cancer,

wherein the antibody-drug conjugate (ADC) comprises an anti-CEACAM5 antibody conjugated to a cytotoxic agent,

wherein the anti-CEACAM5 antibody comprises a HCDR1 having the amino acid sequence of SEQ ID NO: 1, a HCDR2 having the amino acid sequence of SEQ ID NO: 2, a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5, and

wherein the cytotoxic agent is maytansinoid or a maytansinoid analog.

In an embodiment 7, the present disclosure relates to any one of embodiments 1-6, wherein the cancer is selected from the group consisting of colorectal cancer, gastric cancer, gastroesophageal junction cancer, esophageal cancer, lung cancer, uterine cervix cancer, pancreatic cancer, ovarian cancer, thyroid cancer, bladder cancer, endometrial cancer, breast cancer, liver cancer, biliary tract cancer (e.g., cholangiocarcinoma), prostate cancer, and skin cancer.

In an embodiment 8, the present disclosure relates to any one of embodiments 1-7, wherein the cancer is gastric cancer.

In an embodiment 9, the present disclosure relates to any one of embodiments 1-7, wherein the cancer is lung cancer.

In an embodiment 10, the present disclosure relates to embodiment 9, wherein the cancer is non-squamous non-small cell lung cancer (NSQ NSCLC).

In an embodiment 11, the present disclosure relates to embodiment 10, wherein the NSQ NSCLC is advanced or metastatic.

In an embodiment 12, the present disclosure relates to any one of embodiments 1-11, wherein the circulating CEA is measured after performing immunohistochemistry analysis of CEACAM5 expression on tumor cells of the subject.

In an embodiment 13 the present disclosure relates to any one of embodiments 1-12, wherein the subject has negative or low CEACAM5 expression on tumor cells (≥2+ intensity in <1% of cells) as measured by immunohistochemistry.

In an embodiment 14, the present disclosure relates to any one of embodiments 1-12, wherein the subject has moderate CEACAM5 expression on tumor cells (≥2+ intensity in ≥1% and <50% of cells) as measured by immunohistochemistry.

In an embodiment 15, the present disclosure relates to any one of embodiments 1-12, wherein the subject has high CEACAM5 expression on tumor cells (≥2+ intensity in ≥50% of cells) as measured by immunohistochemistry.

In an embodiment 16, the present disclosure relates to any one of embodiments 1-15, wherein prior to treatment the circulating CEA is about ≥20 ng/mL.

In an embodiment 17, the present disclosure relates to any one of embodiments 1-15, wherein prior to treatment the circulating CEA is about ≥25 ng/mL.

In an embodiment 18, the present disclosure relates to any one of embodiments 1-15, wherein prior to treatment the circulating CEA is about ≥30 ng/mL.

In an embodiment 19, the present disclosure relates to any one of embodiments 1-15, wherein prior to treatment the circulating CEA is about ≥35 ng/mL.

In an embodiment 20, the present disclosure relates to any one of embodiments 1-15, wherein prior to treatment the circulating CEA is about ≥40 ng/mL.

In an embodiment 21, the present disclosure relates to any one of embodiments 1-15, wherein prior to treatment the circulating CEA is about ≥45 ng/mL.

In an embodiment 22, the present disclosure relates to any one of embodiments 1-15, wherein prior to treatment the circulating CEA is about ≥50 ng/mL.

In an embodiment 23, the present disclosure relates to any one of embodiments 1-15, wherein prior to treatment the circulating CEA is about ≥55 ng/mL.

In an embodiment 24, the present disclosure relates to any one of embodiments 1-15, wherein prior to treatment the circulating CEA is about ≥60 ng/mL.

In an embodiment 25, the present disclosure relates to any one of embodiments 1-15, wherein prior to treatment the circulating CEA is about ≥61 ng/mL.

In an embodiment 26, the present disclosure relates to any one of embodiments 1-15, wherein prior to treatment the circulating CEA is about ≥62 ng/mL.

In an embodiment 27, the present disclosure relates to any one of embodiments 1-15, wherein prior to treatment the circulating CEA is about ≥63 ng/mL.

In an embodiment 28, the present disclosure relates to any one of embodiments 1-15, wherein prior to treatment the circulating CEA is about ≥64 ng/mL.

In an embodiment 29, the present disclosure relates to any one of embodiments 1-15, wherein prior to treatment the circulating CEA is about ≥65 ng/mL.

In an embodiment 30, the present disclosure relates to any one of embodiments 1-15, wherein prior to treatment the circulating CEA is about ≥66 ng/mL.

In an embodiment 31, the present disclosure relates to any one of embodiments 1-15, wherein prior to treatment the circulating CEA is about ≥67 ng/mL.

In an embodiment 32, the present disclosure relates to any one of embodiments 1-15, wherein prior to treatment the circulating CEA is about ≥68 ng/mL.

In an embodiment 33, the present disclosure relates to any one of embodiments 1-15, wherein prior to treatment the circulating CEA is about ≥69 ng/mL.

In an embodiment 34, the present disclosure relates to any one of embodiments 1-15, wherein prior to treatment the circulating CEA is about ≥70 ng/mL.

In an embodiment 35, the present disclosure relates to any one of embodiments 1-15, wherein prior to treatment the circulating CEA is about ≥71 ng/mL.

In an embodiment 36, the present disclosure relates to any one of embodiments 1-15, wherein prior to treatment the circulating CEA is about ≥72 ng/mL.

In an embodiment 37, the present disclosure relates to any one of embodiments 1-15, wherein prior to treatment the circulating CEA is about ≥73 ng/mL.

In an embodiment 38, the present disclosure relates to any one of embodiments 1-15, wherein prior to treatment the circulating CEA is about ≥74 ng/mL.

In an embodiment 39, the present disclosure relates to any one of embodiments 1-15, wherein prior to treatment the circulating CEA is about ≥75 ng/mL.

In an embodiment 40, the present disclosure relates to any one of embodiments 1-15, wherein prior to treatment the circulating CEA is about ≥76 ng/mL.

In an embodiment 41, the present disclosure relates to any one of embodiments 1-15, wherein prior to treatment the circulating CEA is about ≥77 ng/mL.

In an embodiment 42, the present disclosure relates to any one of embodiments 1-15, wherein prior to treatment the circulating CEA is about ≥78 ng/mL.

In an embodiment 43, the present disclosure relates to any one of embodiments 1-15, wherein prior to treatment the circulating CEA is about ≥79 ng/mL.

In an embodiment 44, the present disclosure relates to any one of embodiments 1-15, wherein prior to treatment the circulating CEA is about ≥80 ng/mL.

In an embodiment 45, the present disclosure relates to any one of embodiments 1-15, wherein prior to treatment the circulating CEA is about ≥80 ng/mL.

In an embodiment 46, the present disclosure relates to any one of embodiments 1-15, wherein prior to treatment the circulating CEA is about ≥85 ng/mL.

In an embodiment 47, the present disclosure relates to any one of embodiments 1-15, wherein prior to treatment the circulating CEA is about ≥90 ng/mL.

In an embodiment 48, the present disclosure relates to any one of embodiments 1-15, wherein prior to treatment the circulating CEA is about ≥95 ng/mL.

In an embodiment 49, the present disclosure relates to any one of embodiments 1-15, wherein prior to treatment the circulating CEA is about ≥100 ng/mL.

In an embodiment 50, the present disclosure relates to any one of embodiments 1-49, wherein the anti-CEACAM5 antibody is tusamitamab.

In an embodiment 51, the present disclosure relates to any one of embodiments 1-50, wherein the ADC is tusamitamab ravtansine.

In an embodiment 52, the present disclosure relates to embodiment 51, wherein the tusamitamab ravtansine is administered in a dose of about ≥100 mg/m² about once every two weeks.

In an embodiment 53, the present disclosure relates to embodiment 51, wherein the tusamitamab ravtansine is administered in a dose of about ≥100 mg/m² about once every three weeks.

In an embodiment 54, the present disclosure relates to any one of embodiments 1-53, further comprising administering to the subject an effective amount of at least one additional agent effective to treat the cancer.

In an embodiment 55, the present disclosure relates to embodiment 54, wherein the additional agent is selected from the group consisting of an immune checkpoint inhibitor (ICI), a platinum-based chemotherapy, pemetrexed, anti-VEGFR2, FOLFOX, FOLFIRI, TAS-102, anti-EGFR, and any combination thereof.

In an embodiment 56, the present disclosure relates to embodiment 55, wherein the ICI is an anti-PD-1 antibody.

In an embodiment 57, the present disclosure relates to embodiment 56, wherein the anti-PD-1 antibody is selected from the group consisting of pembrolizumab, nivolumab, cemiplimab, sintilimab, dostarlimab, and tislelizumab.

In an embodiment 58, the present disclosure relates to embodiment 56, wherein the anti-PD-1 antibody is pembrolizumab.

In an embodiment 59, the present disclosure relates to embodiment 55, wherein the ICI is an anti-PD-L1 antibody.

In an embodiment 60, the present disclosure relates to embodiment 59, wherein the anti-PD-L1 antibody is selected from the group consisting of atezolizumab, avelumab, and durvalumab.

In an embodiment 61, the present disclosure relates to any one of embodiments 1-58, comprising administering to the subject an effective amount of tusamitamab ravtansine and pembrolizumab.

In an embodiment 62, the present disclosure relates to embodiment 61, further comprising administering to the subject an effective amount of a platinum-based chemotherapy.

In an embodiment 63, the present disclosure relates to embodiment 62, wherein the platinum-based chemotherapy is selected from cisplatin and carboplatin.

In an embodiment 64, the present disclosure relates to embodiment 63, further comprising administering to the subject an effective amount of pemetrexed.

### BRIEF DESCRIPTION OF THE DRAWINGS

FIG. 1 is a graph depicting circulating CEACAM5 at baseline centrally assessed and circulating CEA at baseline locally assessed according to embodiments of the present teachings.

FIG. 2 is a schematic depicting study design.

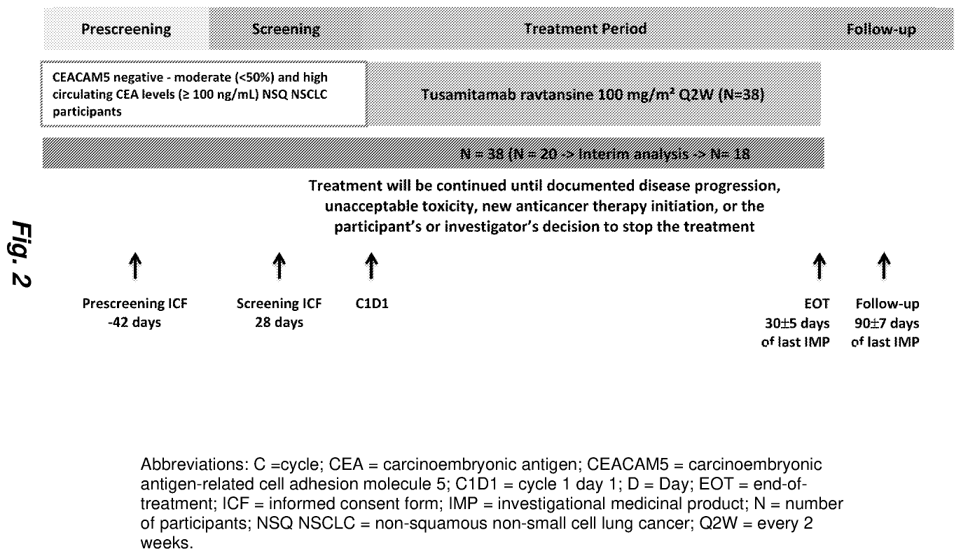

### DETAILED DESCRIPTION

This disclosure provides methods for identifying and treating patients with cancers that express CEACAM5, for example patients who might otherwise not be considered to be likely to respond to treatment because of low or moderate expression of CEACAM5 on tumor tissue as measured by immunohistochemistry (IHC).

It has now been discovered in accordance with this disclosure that patients with cancer and high levels of circulating CEA (e.g., about ≥20, about ≥50, about ≥80, or about ≥100 ng/mL) may be responsive to treatment despite having only low or moderate expression of CEACAM5 on tumor tissue (≥2+ on <50% of tumor cells) as measured by immunohistochemistry.

It has now been discovered in accordance with this disclosure that patients with cancer and high levels of circulating CEA (e.g., about ≥20, about ≥50, about ≥80, or about ≥100 ng/mL) may be responsive to treatment specifically directed to CEACAM5 despite having only low or moderate expression of CEACAM5 on tumor tissue (≥2+ on <50% of tumor cells) as measured by immunohistochemistry.

Tusamitamab ravtansine (CAS Registry No. 2254086-60-5) is an immunoconjugate ADC combining a humanized anti-CEACAM5 antibody (tusamitamab) and the maytansinoid derivative 4 (DM4) [N2′-deacetyl-N2′-(4-methyl-4-mercapto-1-oxopentyl)-maytansine], a potent antimitotic agent that inhibits microtubule assembly. DM4 is covalently bound to the antibody through an optimized linker SPDB [N-succinimidyl 4-(2-pyridyldithio)-butyrate] that is stable in plasma and cleavable inside cells. After binding and internalization in targeted cancer cells, the ADC is degraded, releasing cytotoxic DM4 metabolites.

Tusamitamab ravtansine specifically binds to the A3B3 domain of human CEACAM5 and does not recognize other CEACAMs presenting A or/and B domains in their structure (CEACAM1, CEACAM6, CEACAM7 and CEACAM8). The naked antibody and the ADC bind to recombinant human CEACAM5 with an affinity of ˜0.02 nM (ELISA) and display high affinity for CEACAM5 expressing tumor cells (K_{D}^{APP} 0.24-0.68 nM). Preliminary experiments indicate that tusamitamab ravtansine is devoid of effector activity.

After binding to the CEACAM5 antigen, tusamitamab ravtansine is internalized by the cancer cells via antigen-mediated endocytosis, delivered to lysosomes and degraded into the lysine-linked derivative lysine-SPDB-DM4. The lysine-SPDB-DM4 gets further degraded in DM4 that is subsequently S-methylated to form methyl-DM4 [Me-DM4]; all three metabolites have potent cytotoxic activity through binding to tubulin and inhibition of microtubule polymerization.

As used herein, high CEACAM5 cancer refers to several types including colorectal, lung, gastric, gastroesophageal junction, esophageal, lung, uterine, cervical, pancreatic, ovarian, thyroid, bladder, endometrial, breast, liver, prostate, and skin cancer. In some embodiments, the lung cancer is non-squamous non-small cell lung cancer. In certain embodiments, high CEACAM5 expressers have greater than 2+ intensity in at least 50% of expressing tumor cell population. High CEACAM5 expressers, represent ˜20% of lung cancer.

The ADC was analyzed in a Phase 1/2 study in heavily pre-treated high CEACAM5 expressers for NSCLC. The ADC demonstrated competitive Overall Response Rate (ORR) and Duration of Response (DoR). Most common Adverse Drug Reactions (ADRs) were ocular toxicity (reversible without treatment discontinuation), and minimal hematological/nerve toxicity.

The majority of patients with NSCLC present an advanced stage at the time of diagnosis. These patients have a median overall survival (OS) of 8 to 12 months, and in 2015, a 5-years survival rate of approximately 25%. About 15% to 20% of patients with NSCLC have tumors with key genomic alterations that are amenable to targeted therapy, which include epidermal growth factor receptor (EGFR) mutations and ROS receptor tyrosine kinase 1 (ROS1) and anaplastic lymphoma kinase (ALK) rearrangements.

Until recently the only available treatment option for advanced or metastatic NSQ NSCLC lacking targetable mutations was chemotherapy. Systemic therapy with platinum-based doublet regimens, with or without maintenance therapy, was the current first-line treatment for patients with advanced NSCLC.

More recently, immunotherapy has initiated a new paradigm for the treatment of NSCLC. For example, monoclonal antibodies targeting the programmed death-1 receptor (PD-1)/PD ligand-1 (PD-L1) pathway have emerged as powerful new therapeutic tools in several clinical trials. Four drugs targeting the PD-1 pathway (nivolumab, pembrolizumab, atezolizumab, and cemiplimab) have been approved for the treatment of both chemotherapy-naïve and/or previously treated advanced stage NSCLC, however only a small subset (20% to 30%) of patients responds to these treatments. Despite an improvement in outcomes with these newer lines of therapy, including anti-PD-1/PD-L1 antibodies, the disease often progresses.

The standard second-line treatment for NSCLC has been docetaxel; docetaxel's activity was found to be enhanced by the addition of ramucirumab. There are other available options as single agent treatment such as pemetrexed or gemcitabine, however there remains an unmet medical need for subsequent systemic therapy.

Non-small cell lung cancer is a disease in which malignant (cancer) cells form in the tissues of the lung. Smoking is the major cause of the disease. This is a type of epithelial lung cancer other than small cell lung carcinoma. There are several types of non-small cell lung cancer. Each type of non-small cell lung cancer has different kinds of cancer cells. The cancer cells of each type grow and spread in different ways. The types of non-small cell lung cancer are named for the kinds of cells found in the cancer and how the cells look under a microscope: (1) squamous cell carcinoma: Cancer that begins in squamous cells, which are thin, flat cells that look like fish scales. This is also called epidermoid carcinoma. (2) large cell carcinoma: Cancer that may begin in several types of large cells. And (3) adenocarcinoma: Cancer that begins in the cells that line the alveoli and make substances such as mucus.

Selectively targeting potent cytotoxics to tumor cells using antibody drug conjugates (ADCs) has now been shown to be an effective strategy for the treatment of cancer, as demonstrated by the recent approvals of brentuximab vedotin for the treatment of Hodgkin lymphoma and trastuzumab emtansine (T-DM1) for the treatment of relapsed metastatic HER2+ breast cancer (Young A. et al., 2012J Clin Oncol. 30(18):2183-9; Verma, S. et al., 2012 N Engl J Med. 19:1783-91). Many other malignant diseases with unmet medical needs could benefit from such therapeutic options. The mechanism of action of ADCs begins with its binding to a specific antigen, sufficiently expressed on the tumor cells in order to achieve a selective and efficient internalization of the drug.

Radical surgery (e.g. pneumonectomy, lobectomy, segmentectomy or wedge resection, sleeve resection) is the standard of care for fit stage I NSCLC patients. Adjuvant treatment should be offered only as part of an investigation trial. Stage II and IIIA adjuvant cisplatin-based chemotherapy remains the gold standard for completely resected NSCLC tumors. Other chemotherapeutic agents used in combination with cisplatin, or with each other, may include carboplatin, paclitaxel (TAXOL®), albumin-bound paclitaxel (nab-paclitaxel, ABRAXANE®), docetaxel (TAXOTERE®), Gemcitabine (GEMZAR®), vinorelbine (NAVELBINE®), irinotecan (CAMPTOSAR®), etoposide (VP-16), vinblastine, and pemetrexed (ALIMTA®). Additionally, radiotherapy may be used in patients with N2 lymph nodes. In advanced stage IIIB/IV or inoperable NSCLC pts, treatment may include multiple cycles of cisplatin-based chemotherapy plus a 3rd generation cytotoxic agent or a cytostatic drug (anti-EGFR, anti-VEGFR). (See Zarogoulidis et al., J Thorac Dis. 2013 September; 5(Suppl 4): S389-S396.)

Treatments for cancers, including lung cancers, can include angiogenesis inhibitors, Epidermal growth factor receptor (EGFR) inhibitors, and immune checkpoint inhibitors.

Angiogenesis inhibitors may include, but are not limited to, Axitinib (INLYTA®), Bevacizumab (AVASTIN®), Cabozantinib (COMETRIQ®), Everolimus (AFINITOR®, ZORTRESS®), Lenalidomide (REVLIMID®), Pazopanib (VOTRIENT®), Ramucirumab (CYRAMZA®), Regorafenib (STIVARGA®), Sorafenib (NEXAVAR®), Sunitinib (SUTENT®), Thalidomide (SYNOVIR®, THALOMID®), Vandetanib (CAPRELSA®), and Ziv-aflibercept (ZALTRAP®).

Epidermal growth factor receptor (EGFR) inhibitors may include, but are not limited to, gefitinib (IRESSA®), erlotinib (TARCEVA®), lapatinib (TYKERB®), cetuximab (ERBITUX®), neratinib (NERLYNX®), osimertinib (TAGRISSO®), panitumumab (VECTIBIX®), vandetanib (CAPRELSA®), necitumumab (PROTRAZZA®), and dacomitinib (VIZIMPRO®).

Immune checkpoint inhibitors may include, but are not limited to, Programmed Death 1 receptor (PD-1) binding agents (e.g., pembrolizumab (KEYTRUDA®), nivolumab (OPDIVO®), cemiplimab (LIBTAYO®), Programmed Death-ligand 1 (PD-L1) binding agents (e.g., atezolizumab (TECENTRIQ®), avelumab (BAVENCIO®), durvalumab (IMFINZI®)), CTLA-4 binding agents (e.g. ipilimumab (YERVOY®)), OX40 or OX40L binding agents, Adenosine A2A receptor binding agents, B7-H3 binding agents, B7-H4 binding agents, BTLA binding agents, Indoleamine 2,3-dioxygenase binding agents, Killer-cell Immunoglobulin-like Receptor (KIR) binding agents, Lymphocyte Activation Gene-3 (LAG-3) binding agents, nicotinamide adenine dinucleotide phosphate NADPH oxidase isoform (NOX2) binding agents, T-cell Immunoglobulin domain and Mucin domain 3 (TIM-3) binding agents, V-domain Ig suppressor of T cell activation (VISTA) binding agents, Glucocorticoid-Induced TNFR family Related gene (GITR) binding agents, and Sialic acid-binding immunoglobulin-type lectin 7 (SIGLEC7) binding agents.

### CEACAM5 and Indications

Carcinoembryonic antigen (CEA) is a glycoprotein involved in cell adhesion. CEA was first identified in 1965 (Gold and Freedman, J Exp Med, 121, 439, 1965) as a protein normally expressed by fetal gut during the first six months of gestation, and found in cancers of the pancreas, liver and colon. The CEA family belongs to the immunoglobulin superfamily. The CEA family, which consists of 18 genes, is sub-divided in two sub-groups of proteins: the carcinoembryonic antigen-related cell adhesion molecule (CEACAM) sub-group and the pregnancy-specific glycoprotein subgroup (Kammerer & Zimmermann, BMC Biology 2010, 8:12).

In humans, the CEACAM sub-group consists of 7 members: CEACAM1, CEACAM3, CEACAM4, CEACAM5, CEACAM6, CEACAM7, CEACAM8. Numerous studies have shown that CEACAM5, identical to the originally identified CEA, is highly expressed on the surface of colorectal, gastric, lung, breast, prostate, ovary, cervix, and bladder tumor cells and weakly expressed in few normal epithelial tissues such as columnar epithelial and goblet cells in colon, mucous neck cells in the stomach and squamous epithelial cells in esophagus and cervix (Hammarstrom et al, 2002, in “Tumor markers, Physiology, Pathobiology, Technology and Clinical Applications” Eds. Diamandis E. P. et al., AACC Press, Washington pp 375). Thus, CEACAM5 may constitute a therapeutic target suitable for tumor-specific targeting approaches, such as immunoconjugates. The extracellular domains of CEACAM family members are composed of repeated immunoglobulin-like (Ig-like) domains which have been categorized in 3 types, A, B and N, according to sequence homologies. CEACAM5 contains seven such domains, namely N, A1, B1, A2, B2, A3 and B3.

CEACAM5 A1, A2 and A3 domains, on one hand, and B1, B2 and B3 domains, on the other hand, show high sequence homologies, the A domains of human CEACAM5 presenting from 84 to 87% pairwise sequence similarity, and the B domains from 69 to 80%. Furthermore, other human CEACAM members presenting A and/or B domains in their structure, namely CEACAM1, CEACAM6, CEACAM7 and CEACAM8, show homology with human CEACAM5. In particular, the A and B domains of human CEACAM6 protein display sequence homologies with A1 and A3 domains, and any of B1 to B3 domains of human CEACAM5, respectively, which are even higher than observed among the A domains and the B domains of human CEACAM5.

### Anti-CEACAM5 Antibody:

Numerous anti-CEA antibodies were generated in view of CEA-targeted diagnostic or therapeutic purposes. Specificity towards related antigens has always been mentioned as a concern in this field, as an example by Sharkey et al (1990, Cancer Research 50, 2823). Due to the above-mentioned homologies some of previously described antibodies may demonstrate binding to repetitive epitopes of CEACAM5 present in the different immunoglobulin domains show cross-reactivity to other CEACAM members such as CEACAM1, CEACAM6, CEACAM7, or CEACAM8, lacking specificity to CEACAM5. The specificity of the anti-CEACAM5 antibody is desired in view of CEA-targeted therapies such that it binds to human CEACAM5-expressing tumor cells but does not bind to some normal tissues expressing the others CEACAM members. It is noteworthy that CEACAM1, CEACAM6 and CEACAM8 have been described as expressed by neutrophils of human and non-human primates (Ebrahimmnejad et al, 2000, Exp Cell Res, 260, 365; Zhao et al, 2004, J Immunol Methods 293, 207; Strickland et al, 2009 J Pathol, 218, 380) where they have been shown to regulate granulopoiesis and to play a role in immune response.

The ADC tusamitamab ravtansine has been shown to be capable of being internalized into cells expressing CEACAM5 after binding, and to induce cytotoxic activity on tumor cells in vitro. Tusamitamab ravtansine is also able to markedly inhibit tumor growth in vivo in mice bearing human primary colon and stomach tumors. See WO 2014/079886, which is incorporated herein in its entirety.

As used herein, the term “about” in quantitative terms refers to plus or minus 10% of the value it modifies (rounded up to the nearest whole number if the value is not sub-dividable, such as a number of molecules or nucleotides). For example, the phrase “about 100 mg” would encompass 90 mg to 110 mg, inclusive; the phrase “about 2500 mg” would encompass 2250 mg to 2750 mg. When applied to a percentage, the term “about” refers to plus or minus 10% relative to that percentage. For example, the phrase “about 20%” would encompass 18-22% and “about 80%” would encompass 72-88%, inclusive. Moreover, where “about” is used herein in conjunction with a quantitative term it is understood that in addition to the value plus or minus 10%, the exact value of the quantitative term is also contemplated and described. For example, the term “about 23%” expressly contemplates, describes, and includes exactly 23%.

It is to be noted that the term “a” or “an” entity refers to one or more of that entity; for example, “a symptom,” is understood to represent one or more symptoms. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

As used herein “CEACAM5” designates the “carcinoembryonic antigen-related cell adhesion molecule 5”, also known as “CD66e” (Cluster of Differentiation 66e) or CEA. CEACAM5 is a glycoprotein involved in cell adhesion. CEACAM5 is highly expressed for example on the surface of colorectal cancer, gastric cancer, gastroesophageal junction cancer, esophageal cancer, lung, and uterine tumor cells.

A reference sequence of full length human CEACAM5, including signal peptide (positions 1-34) and propeptide (positions 686-702), is available from the GenBank database under accession number AAA51967.1. Five nonsynonymous SNPs have been identified with a frequency higher than 2% in Caucasian population, four of them being localised in the N domain (at positions 80, 83, 112, 113), the last one in the A2 domain (at position 398) of human CEACAM5.

It is understood that wherever aspects or embodiments are described herein with the language “comprising,” otherwise analogous aspects described in terms of “consisting of” and/or “consisting essentially of” are also provided.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide, or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, VHH or NANOBODY® (e.g., monovalent VHHs, and bivalent VHH), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH—VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody include: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may in various embodiments consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody may in various embodiments comprise a homo-dimer or hetero-dimer (or other multimer) of any of the variable and constant domain configurations listed above in non-covalent association with one another and/or with one or more monomeric VH or VL domain (e.g., by disulfide bond(s)).

In some embodiments, the antibody or antibody fragment for use in the method of the disclosure may be a multispecific antibody, which may be specific for different epitopes of one target polypeptide or may contain antigen-binding domains specific for epitopes of more than one target polypeptide. An exemplary bi-specific antibody format that can be used in the context of the present disclosure involves the use of a first immunoglobulin (Ig) CH3 domain and a second Ig CH3 domain, wherein the first and second Ig CH3 domains differ from one another by at least one amino acid, and wherein at least one amino acid difference reduces binding of the bispecific antibody to Protein A as compared to a bi-specific antibody lacking the amino acid difference. In one embodiment, the first Ig CH3 domain binds Protein A and the second Ig CH3 domain contains a mutation that reduces or abolishes Protein A binding such as an H95R modification (by IMGT exon numbering; H435R by EU numbering). The second CH3 may further comprise an Y96F modification (by IMGT; Y436F by EU). Further modifications that may be found within the second CH3 include: D16E, L18M, N44S, K52N, V57M, and V82I (by IMGT; D356E, L358M, N384S, K392N, V397M, and V422I by EU) in the case of IgGI antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies. Variations on the bi-specific antibody format described above are contemplated within the scope of the present disclosure. Any multispecific antibody format, including the exemplary bispecific antibody formats disclosed herein, may in various embodiments be adapted for use in the context of an antigen-binding fragment of an anti-CEACAM5 antibody using routine techniques available in the art.

The CEACAM5 antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present disclosure includes antibodies, and antigen-binding fragments thereof, which are derived from any of the amino acid sequences disclosed herein, wherein one or more amino acids within one or more framework and/or CDR regions are back-mutated to the corresponding germline residue(s) or to a conservative amino acid substitution (natural or non-natural) of the corresponding germline residue(s) (such sequence changes are referred to herein as “germline back-mutations”). A person of ordinary skill in the art, starting with the heavy and light chain variable region sequences disclosed herein, can easily produce numerous antibodies and antigen-binding fragments which comprise one or more individual germline back-mutations or combinations thereof. In certain embodiments, all of the framework residues and/or CDR residues within the VH and/or VL domains are mutated back to the germline sequence. In other embodiments, only certain residues are mutated back to the germline sequence, e.g., only the mutated residues found within the first 8 amino acids of FR1 or within the last 8 amino acids of FR4, or only the mutated residues found within CDR1, CDR2 or CDR3. Furthermore, the antibodies of the present disclosure may contain any combination of two or more germline back-mutations within the framework and/or CDR regions, i.e., wherein certain individual residues are mutated back to the germline sequence while certain other residues that differ from the germline sequence are maintained. Once obtained, antibodies and antigen-binding fragments that contain one or more germline back-mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody”, as used herein, is intended to include antibodies having variable and constant regions derived from human germline immunoglobulin sequences. The human antibodies featured in the disclosure may in various embodiments nonetheless include amino acid residues not encoded by human germline immunoglobulin sequences (e.g., mutations introduced by random or site-specific mutagenesis in vitro or by somatic mutation in vivo), for example in the CDRs and in some embodiments CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al., (1992) Nucl. Acids Res. 20:6287-6295, incorporated herein by reference in its entirety) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Human antibodies can exist in two forms that are associated with hinge heterogeneity. In an embodiment, an immunoglobulin molecule comprises a stable four chain construct of approximately 150-160 kDa in which the dimers are held together by an interchain heavy chain disulfide bond. In another embodiment, the dimers are not linked via inter-chain disulfide bonds and a molecule of about 75-80 kDa is formed composed of a covalently coupled light and heavy chain (half-antibody). These embodiments/forms have been extremely difficult to separate, even after affinity purification.

The term “humanised antibody” or “humanized antibody” refers to an antibody which is wholly or partially of non-human origin and which has been modified to replace certain amino acids, for instance in the framework regions of the VH and VL domains, in order to avoid or minimize an immune response in humans. The constant domains of a humanized antibody are most of the time human CH and CL domains.

Numerous methods for humanisation/humanization of an antibody sequence are known in the art; see e.g. the review by Almagro & Fransson (2008) Front Biosci. 13: 1619-1633. One commonly used method is CDR grafting, or antibody reshaping, which involves grafting of the CDR sequences of a donor antibody, generally a mouse antibody, into the framework scaffold of a human antibody of different specificity. Since CDR grafting may reduce the binding specificity and affinity, and thus the biological activity, of a CDR grafted non-human antibody, back mutations may be introduced at selected positions of the CDR grafted antibody in order to retain the binding specificity and affinity of the parent antibody. Identification of positions for possible back mutations can be performed using information available in the literature and in antibody databases. Amino acid residues that are candidates for back mutations are typically those that are located at the surface of an antibody molecule, while residues that are buried or that have a low degree of surface exposure will not normally be altered. An alternative humanization technique to CDR grafting and back mutation is resurfacing, in which non-surface exposed residues of non-human origin are retained, while surface residues are altered to human residues. Another alternative technique is known as “guided selection” (Jespers et al. (1994) Biotechnology 12, 899) and can be used to derive from a murine antibody a fully human antibody conserving the epitope and binding characteristics of the parental antibody.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al., (1993) Molecular Immunology 30:105, incorporated by reference in its entirety) to levels typically observed using a human IgGI hinge. The instant disclosure encompasses in various embodiments antibodies having one or more mutations in the hinge, CH2 or CH3 region which may be desirable, for example, in production, to improve the yield of the desired antibody form.

An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” In various embodiments, the isolated antibody also includes an antibody in situ within a recombinant cell. In other embodiments, isolated antibodies are antibodies that have been subjected to at least one purification or isolation step. In various embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” CEACAM5, as used herein, includes antibodies that bind CEACAM5 or portion thereof with a KD of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or about 0.5 nM, as measured in a surface plasmon resonance assay. Specific binding can also be characterized by a dissociation constant of at least about 1×10⁻⁶ M or smaller. In other embodiments, the dissociation constant is at least about 1×10⁻⁷ M, 1×10⁻⁸ M, or 1×10⁻⁹ M. An isolated antibody that specifically binds human CEACAM5 may, however, have cross-reactivity to other antigens, such as CEACAM5 molecules from other (non-human) species.

The term “surface plasmon resonance”, as used herein, refers to an optical phenomenon that allows for the analysis of real-time interactions by detection of alterations in protein concentrations within a biosensor matrix, for example using the BIACORE™ system (Biacore Life Sciences division of GE Healthcare, Piscataway, NJ).

The term “KD”, as used herein, is intended to refer to the equilibrium dissociation constant of an antibody-antigen interaction.

“Affinity” is defined, in theory, by the equilibrium association between the whole antibody and the antigen. It can be experimentally assessed by a variety of known methods, such as measuring association and dissociation rates with surface plasmon resonance or measuring the EC50 (or apparent KD) in an immunochemical assay (ELISA, FACS). In these assays, the EC50 is the concentration of the antibody which induces a response halfway between the baseline and maximum after some specified exposure time on a defined concentration of antigen by ELISA (enzyme-linked immuno-sorbent assay) or cell expressing the antigen by FACS (Fluorescence Activated Cell Sorting).

A monoclonal antibody binding to antigen 1 (Ag1) is “cross-reactive” to antigen 2 (Ag2) when the EC50s are in a similar range for both antigens. In the present application, a monoclonal antibody binding to Ag1 is cross-reactive to Ag2 when the ratio of affinity of Ag2 to affinity of Ag1 is equal or less than 10 (for instance 5, 2, 1 or 0.5), affinities being measured with the same method for both antigens.

Affinity for human CEACAM5 or for Macaca fascicularis CEACAM5 may be determined as the EC50 value in an ELISA using soluble recombinant CEACAM5 as capture antigen.

The antibody of the disclosure may also have an apparent dissociation constant (apparent KD), as may be determined by FACS analysis on tumor cell line MKN45 (DSMZ, ACC 409) or on xenograft tumor cells deriving from patient (CR-IGR-034P available from Oncodesign Biotechnology, tumor collection CReMEC), which is ≤25 nM, for instance 20 nM, ≤10 nM, ≤5 nM, ≤3 nM or ≤1 nM. The apparent KD (K_{D}^{APP}) may be within the range 0.01-20 nM, or may be within the range 0.1-20 nM, 0.1-10 nM, or 0.1-5 nM.

Additionally, antibodies according to the disclosure have been shown to be able to detect CEACAM5 expression by immunohistochemistry in frozen and formalin-fixed and paraffin embedded (FFPE) tissue sections.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The anti-CEACAM5 antibodies useful for the methods described herein may in various embodiments include one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present disclosure includes in various embodiments methods involving the use of antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). Numerous antibodies and antigen-binding fragments may be constructed which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies may contain any combination of two or more germline mutations within the framework and/or CDR regions, e.g., wherein certain individual residues are mutated to the corresponding residue of a certain germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the present disclosure.

The present disclosure also includes methods involving the use of anti-CEACAM5 antibodies comprising variants of any of the HCVR, LCVR, and/or CDR amino acid sequences disclosed herein having one or more conservative substitutions. For example, the present disclosure includes the use of anti-CEACAM5 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

According to the present disclosure, the anti-CEACAM5 antibody, or antigen-binding fragment thereof, in various embodiments comprises a heavy chain variable region (HCVR), light chain variable region (LCVR), and/or complementarity determining regions (CDRs) comprising any of the amino acid sequences of the anti-CEACAM5 antibodies described in Intl. Patent Pub. No. WO 2014/079886 A1, incorporated herein by reference in its entirety.

Amino acid sequence modification(s) of the antibodies described herein are contemplated. For example, it may be desirable to improve the binding affinity and/or other biological properties of the antibody. It is known that when a humanised antibody is produced by simply grafting only CDRs in VH and VL of an antibody derived from a non-human animal in FRs of the VH and VL of a human antibody, the antigen binding activity may be reduced in comparison with that of the original antibody derived from a non-human animal. It is considered that several amino acid residues of the VH and VL of the non-human antibody, not only in CDRs but also in FRs, may be directly or indirectly associated with the antigen binding activity. Hence, substitution of these amino acid residues with different amino acid residues derived from FRs of the VH and VL of the human antibody would reduce the binding activity. In order to solve the problem, in human antibodies grafted with non-human CDRs, attempts have to be made to identify, among amino acid sequences of the FR of the VH and VL of human antibodies, an amino acid residue which is directly associated with binding of the antibody, or which interacts with an amino acid residue of a CDR, or which maintains the three-dimensional structure of the antibody and which is directly associated with binding to the antigen. The reduced antigen binding activity could be increased by replacing the identified amino acids with amino acid residues of the original antibody derived from a non-human animal.

Modifications and changes may be made in the structure of the antibodies of the present disclosure, and in the DNA sequences encoding them, and still result in a functional antibody or polypeptide with desirable characteristics.

A further object of the present disclosure also encompasses function-conservative variants of the polypeptides of the present disclosure. For example, certain amino acids may be substituted by other amino acids in a protein structure without appreciable loss of activity. Since the interactive capacity and nature of a protein define its biological functional activity, certain amino acid substitutions can be made in a protein sequence, and of course in its DNA encoding sequence, while nevertheless obtaining a protein with like properties. It is thus contemplated that various changes may be made in the antibodies sequences of the disclosure, or corresponding DNA sequences which encode said polypeptides, without appreciable loss of their biological activity. It is known in the art that certain amino acids may be substituted by other amino acids having a similar hydropathic index or score and still result in a protein with similar biological activity, i.e. still obtain a biological functionally equivalent protein. It is also possible to use well-established technologies, such as alanine-scanning approaches, to identify, in an antibody or polypeptide of the disclosure, all the amino acids that can be substituted without significant loss of binding to the antigen. Such residues can be qualified as neutral, since they are not involved in antigen binding or in maintaining the structure of the antibody. One or more of these neutral positions can be substituted by alanine or by another amino acid can without changing the main characteristics of the antibody or polypeptide of the disclosure.

Neutral positions can be seen as positions where any amino acid substitution could be incorporated to the antibodies. Indeed, in the principle of alanine-scanning, alanine is chosen since it this residue does not carry specific structural or chemical features. It is generally admitted that if an alanine can be substituted for a specific amino acid without changing the properties of a protein, many other, if not all amino acid substitutions are likely to be also neutral. In the opposite case where alanine is the wild-type amino acid, if a specific substitution can be shown as neutral, it is likely that other substitutions would also be neutral. As outlined above, amino acid substitutions are generally therefore based on the relative similarity of the amino acid side-chain substituents, for example, their hydrophobicity, hydrophilicity, charge, size, and the like. Exemplary substitutions which take any of the foregoing characteristics into consideration are well known to those of skill in the art and include: arginine and lysine; glutamate and aspartate; serine and threonine; glutamine and asparagine; and valine, leucine and isoleucine.

It may be also desirable to modify the antibody of the disclosure with respect to effector function, e.g. so as to enhance antigen-dependent cell-mediated cytotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing inter-chain disulfide bond formation in this region. The homodimeric antibody thus generated may have improved internalization capability and/or increased complement-mediated cell killing and/or antibody-dependent cellular cytotoxicity (ADCC) (Caron P C. et al. 1992; and Shopes B. 1992).

Another type of amino acid modification of the antibody of the disclosure may be useful for altering the original glycosylation pattern of the antibody, i.e. by deleting one or more carbohydrate moieties found in the antibody, and/or adding one or more glycosylation sites that are not present in the antibody. The presence of either of the tripeptide sequences asparagine-X-serine, and asparagine-X-threonine, where X is any amino acid except proline, creates a potential glycosylation site. Addition or deletion of glycosylation sites to the antibody is conveniently accomplished by altering the amino acid sequence such that it contains one or more of the above-described tripeptide sequences (for N-linked glycosylation sites).

Another type of modification involves the removal of sequences identified, either in silico or experimentally, as potentially resulting in degradation products or heterogeneity of antibody preparations. As examples, deamidation of asparagine and glutamine residues can occur depending on factors such as pH and surface exposure. Asparagine residues are particularly susceptible to deamidation, primarily when present in the sequence Asn-Gly, and to a lesser extent in other dipeptide sequences such as Asn-Ala. When such a deamidation site, in particular Asn-Gly, is present in an antibody or polypeptuide of the disclosure, it may therefore be desirable to remove the site, typically by conservative substitution to remove one of the implicated residues. Such substitutions in a sequence to remove one or more of the implicated residues are also intended to be encompassed by the present disclosure.

Another type of covalent modification involves chemically or enzymatically coupling glycosides to the antibody. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. For example, such methods are described in WO87/05330.

Removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Sojahr H. et al. (1987) and by Edge, A S. et al. (1981). Enzymatic cleavage of carbohydrate moieties on antibodies can be achieved by the use of a variety of endo- and exo-glycosidases as described by Thotakura, N R. et al. (1987).

Another type of covalent modification of the antibody comprises linking the antibody to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192; or 4,179,337.

In an embodiment, the anti-CEACAM5 antibody is Tusamitamab (CAS Registry No. 2349294-95-5).

Tusamitamab comprises a HCDR1 having the amino acid sequence of SEQ ID NO: 1, a HCDR2 having the amino acid sequence of SEQ ID NO: 2, a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5.

HCDR1
(SEQ ID NO: 1)
GFVFSSYD
HCDR2
(SEQ ID NO: 2)
ISSGGGIT
HCDR3
(SEQ ID NO: 3)
AAHYFGSSGPFAY
LCDR1
(SEQ ID NO: 4)
ENIFSY
LCDR2
NTR
LCDR3
(SEQ ID NO: 5)
QHHYGTPFT

Tusamitamab comprises a variable domain of a heavy chain (VH) having the amino acid sequence of SEQ ID NO: 6 and a variable domain of a light chain (VL) having the amino acid sequence of SEQ ID NO: 7.

(SEQ ID NO: 6)
EVQLQESGPGLVKPGGSLSLSCAASGFVFSSYDMSWVRQTPERGLEWVA
YISSGGGITYAPSTVKGRFTVSRDNAKNTLYLQMNSLTSEDTAVYYCAA
HYFGSSGPFAYVVGQGTLVTVSS
(SEQ ID NO: 7)
DIQMTQSPASLSASVGDRVTITCRASENIFSYLAWYQQKPGKSPKLLVY
NTRTLAEGVPSRFSGSGSGTDFSLTISSLQPEDFATYYCQHHYGTPFTF
GSGTKLEI

Tusamitamab comprises a heavy chain (HC) having the amino acid sequence of SEQ ID NO: 8 and a light chain (LC) having the amino acid sequence of SEQ ID NO: 9.

(SEQ ID NO: 8)
EVQLQESGPGLVKPGGSLSLSCAASGFVFSSYDMSWVRQTPERGLEWVA
YISSGGGITYAPSTVKGRFTVSRDNAKNTLYLQMNSLTSEDTAVYYCAA
HYFGSSGPFAYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNYNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRWSVLTVLHQDWLNGKEYKCKVSNKALPAPIEKTISKAKG
QPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQK
SLSLSPG
(SEQ ID NO: 9)
DIQMTQSPASLSASVGDRVTITCRASENIFSYLAWYQQKPGKSPKLLVY
NTRTLAEGVPSRFSGSGSGTDFSLTISSLQPEDFATYYCQHHYGTPFTF
GSGTKLEIKRTVAAPSVFIFPPSDEQLKSGTASWCLLNNFYPREAKVQW
KVDNALQSGNSQESVTEQDSKDSTYSLSSTLTLSKADYEKHKVYACEVT
HQGLSSPVTKSFNRGEC

### Cytotoxic Payload and Immunoconjugates

The present disclosure also includes cytotoxic conjugates, or immunoconjugates, or antibody-drug conjugates, or conjugates. As used herein, all these terms have the same meaning and are interchangeable.

Accordingly, the disclosure relates to “immunoconjugates” comprising an antibody of the disclosure (e.g. anti-CEACAM5 antibody) linked or conjugated to at least one growth inhibitory agent, such as a cytotoxic agent or a radioactive isotope.

A “growth inhibitory agent”, or “anti-proliferative agent”, which can be used indifferently, refers to a compound or composition which inhibits growth of a cell, especially tumour cell, either in vitro or in vivo.

The term “cytotoxic agent” as used herein refers to a substance that inhibits or prevents the function of cells and/or causes destruction of cells. The term “cytotoxic agent” is intended to include chemotherapeutic agents, enzymes, antibiotics, and toxins such as small molecule toxins or enzymatically active toxins of bacterial, fungal, plant or animal origin, including fragments and/or variants thereof, and the various antitumor or anticancer agents disclosed below. In some embodiments, the cytotoxic agent is a taxoid, vincas, a maytansinoid or maytansinoid analog such as DM1 or DM4, a small drug, a tomaymycin or pyrrolobenzodiazepine derivative, a cryptophycin derivative, a leptomycin derivative, an auristatin or dolastatin analog, a prodrug, topoisomerase II inhibitors, a DNA alkylating agent, an anti-tubulin agent, a CC-1065 or CC-1065 analog.

As used herein “maytansinoids” denotes maytansinoids and maytansinoid analogs. Maytansinoids are drugs that inhibit microtubule formation and that are highly toxic to mammalian cells.

Examples of suitable maytansinoids include maytansinol and maytansinol analogs.

Examples of suitable maytansinol analogues include those having a modified aromatic ring and those having modifications at other positions. Such suitable maytansinoids are disclosed in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 6,333,410; 5,475,092; 5,585,499; and 5,846,545.

Examples of suitable analogues of maytansinol having a modified aromatic ring include:
(1)C-19-dechloro (U.S. Pat. No. 4,256,746) (prepared by LAH reduction of ansamytocin P2);
(2)C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and
(3)C-20-demethoxy, C-20-acyloxy (—OCOR), +/−dechloro (U.S. Pat. No. 4,294,757) (prepared by acylation using acyl chlorides).

Examples of suitable analogues of maytansinol having modifications of other positions include:
(1)C-9-SH (U.S. Pat. No. 4,424,219) (prepared by the reaction of maytansinol with H₂S or P2S5);
(2)C-14-alkoxymethyl (demethoxy/CH₂OR) (U.S. Pat. No. 4,331,598);
(3)C-14-hydroxymethyl or acyloxymethyl (CH₂OH or CH₂OAc) (U.S. Pat. No. 4,450,254) (prepared from Nocardia);
(4)C-15-hydroxy/acyloxy (U.S. Pat. No. 4,364,866) (prepared by the conversion of maytansinol by Streptomyces);
(5)C-15-methoxy (U.S. Pat. Nos. 4,313,946 and 4,315,929) (isolated from Trewia nudiflora);
(6)C-18-N-demethyl (U.S. Pat. Nos. 4,362,663 and 4,322,348) (prepared by the demethylation of maytansinol by Streptomyces); and
(7) 4,5-deoxy (U.S. Pat. No. 4,371,533) (prepared by the titanium trichloride/LAH reduction of maytansinol).

In an embodiment of the disclosure, the cytotoxic conjugates of the present disclosure utilize the thiol-containing maytansinoid (DM1), formally termed N2′-deacetyl-N2′-(3-mercapto-1-oxopropyl)-maytansine, as the cytotoxic agent. DM1 is represented by the following structural formula (I):

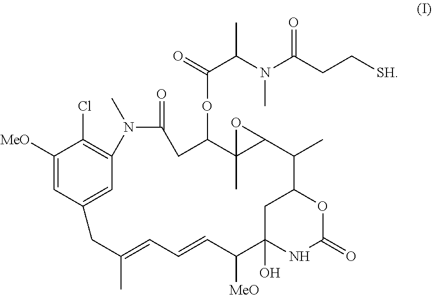 [SMILES: COc1cc2cc(c1Cl)N(C)C(=O)CC(OC(=O)C(C)N(C)C(=O)CCS)C1(C)OC1C(C)C1CC(O)(NC(=O)O1)C(OC)/C=C/C=C(\C)C2]

In another embodiment, the cytotoxic conjugates of the present disclosure utilize the thiol-containing maytansinoid DM4, formally termed N2′-deacetyl-N-2′(4-methyl-4-mercapto-1-oxopentyl)-maytansine, as the cytotoxic agent. DM4 is represented by the following structural formula (II):

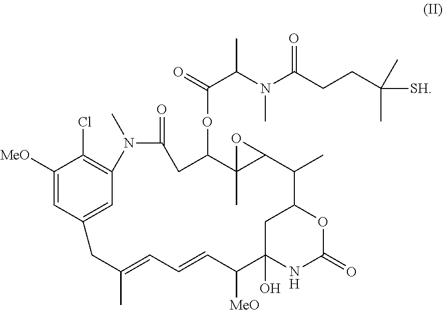 [SMILES: COc1cc2cc(c1Cl)N(C)C(=O)CC(OC(=O)C(C)N(C)C(=O)CCC(C)(C)S)C1(C)OC1C(C)C1CC(O)(NC(=O)O1)C(OC)/C=C/C=C(\C)C2]

In further embodiments of the disclosure, other maytansines, including thiol and disulfide-containing maytansinoids bearing a mono or di-alkyl substitution on the carbon atom bearing the sulfur atom, may be used. These include a maytansinoid having, at C-3, C-14 hydroxymethyl, C-15 hydroxy, or C-20 desmethyl, an acylated amino acid side chain with an acyl group bearing a hindered sulfhydryl group, wherein the carbon atom of the acyl group bearing the thiol functionality has one or two substituents, said substituents being CH₃, C₂H₅, linear or branched alkyl or alkenyl having from 1 to 10 reagents and any aggregate which may be present in the solution.

Examples of these cytotoxic agents and of methods of conjugation are further given in the application WO2008/010101 which is incorporated by reference.

The term “radioactive isotope” is intended to include radioactive isotopes suitable for treating cancer, such as At²¹¹, Bi²¹², Er¹⁶⁹, I¹³¹, I¹²⁵, Y⁹⁰, In¹¹¹, P³², Re¹⁸⁶, Re¹⁸⁸, Sm¹⁵³, Sr⁸⁹, and radioactive isotopes of Lu. Such radioisotopes generally emit mainly beta-radiation. In an embodiment the radioactive isotope is alpha-emitter isotope, more precisely Thorium 227 which emits alpha-radiation. The immunoconjugates according to the present disclosure can be prepared as described in the application WO2004/091668.

The immunoconjugates according to the present disclosure can be prepared as described in the application WO 2004/091668, the entire content of which is incorporated herein by reference.

In some embodiments, the antibodies of the present disclosure are covalently attached, directly or via a cleavable or non-cleavable linker, to at least one growth inhibitory agent.

“Linker”, as used herein, means a chemical moiety comprising a covalent bond or a chain of atoms that covalently attaches a polypeptide (e.g., an antibody) to a drug (or prodrug) moiety. Suitable linkers are well known in the art and include disulfide groups, thioether groups, acid labile groups, photolabile groups, peptidase labile groups and esterase labile groups. Exemplary linkers include, but are not limited to, heterobifunctional crosslinking reagents such as N-succinimidyl pyridyldithiobutyrate (SPDB), butanoic acid 4-[(5-nitro-2-pyridinyl)dithio]-2,5-dioxo-1-pyrrolidinyl ester (nitro-SPDB), 4-(Pyridin-2-yldisulfanyl)-2-sulfo-butyric acid (sulfo-SPDB), N-succinimidyl (2-pyridyldithio) propionate (SPDP), succinimidyl (N-maleimidomethyl) cyclohexane-1-carboxylate (SMCC), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl)-hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al (1987). Carbon labeled 1-isothiocyanatobenzyl methyldiethylene triaminepentaacetic acid (MX-DTPA) is an exemplary chelating agent for conjugation of radionucleotide to the antibody (WO 94/11026).

The linker may be a “cleavable linker” facilitating release of the cytotoxic agent or growth inhibitory agent in the cell. For example, an acid-labile linker, a peptidase-sensitive linker, an esterase labile linker, a photolabile linker or a disulfide-containing linker (See e.g. U.S. Pat. No. 5,208,020) may be used. The linker may be also a “non-cleavable linker” (for example SMCC linker) that might lead to better tolerance in some cases.

Alternatively, a fusion protein comprising the antibody of the disclosure and a cytotoxic or growth inhibitory polypeptide may be made, by recombinant techniques or peptide synthesis. The length of DNA may comprise respective regions encoding the two portions of the conjugate either adjacent one another or separated by a region encoding a linker peptide which does not destroy the desired properties of the conjugate.

The antibodies of the present disclosure may also be used in Dependent Enzyme Mediated Prodrug Therapy by conjugating the polypeptide to a prodrug-activating enzyme which converts a prodrug (e.g. a peptidyl chemotherapeutic agent, see WO81/01145) to an active anti-cancer drug (See, for example, WO 88/07378 and U.S. Pat. No. 4,975,278). The enzyme component of the immunoconjugate useful for ADEPT includes any enzyme capable of acting on a prodrug in such a way so as to convert it into its more active, cytotoxic form. Enzymes that are useful in the method of this disclosure include, but are not limited to, alkaline phosphatase useful for converting phosphate-containing prodrugs into free drugs; arylsulfatase useful for converting sulfate-containing prodrugs into free drugs; cytosine deaminase useful for converting non-toxic fluorocytosine into the anticancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as O-galactosidase and neuraminidase useful for converting glycosylated prodrugs into free drugs; P-lactamase useful for converting drugs derivatized with P-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. The enzymes can be covalently bound to the polypeptides of the disclosure by techniques well known in the art such as the use of the heterobifunctional crosslinking reagents discussed above.

According to an embodiment, in the conjugate of the disclosure, the growth inhibitory agent is a maytansinoid, in an embodiment DM1 or DM4.

In said conjugate, the antibody is conjugated to said at least one growth inhibitory agent by a linking group. In an embodiment said linking group is a cleavable or a non-cleavable linker, such as SPDB, sulfo-SPDB, or SMCC.

The conjugate may be selected from the group consisting of:

an antibody-SPDB-DM4 conjugate of formula (III)

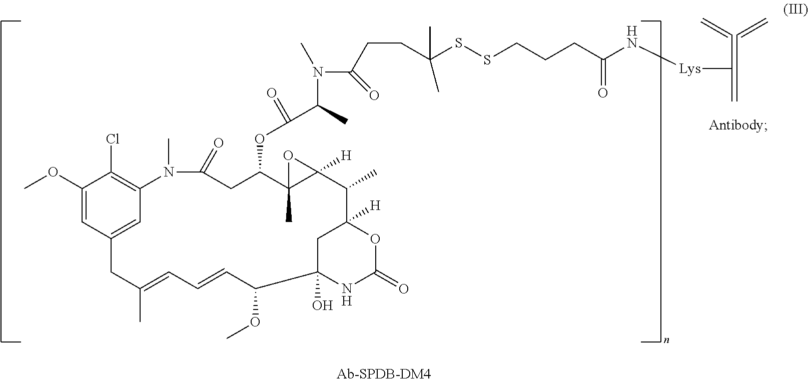

an antibody-sulfo-SPDB-DM4 conjugate of formula (IV)

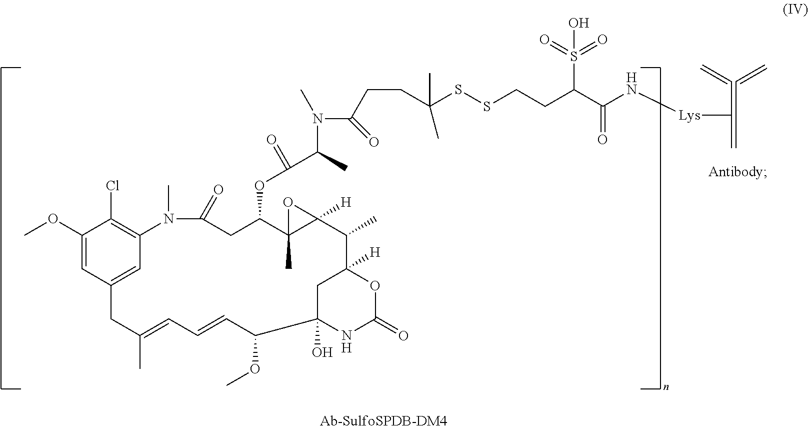

and an antibody-SMCC-DM1 conjugate of formula (V)

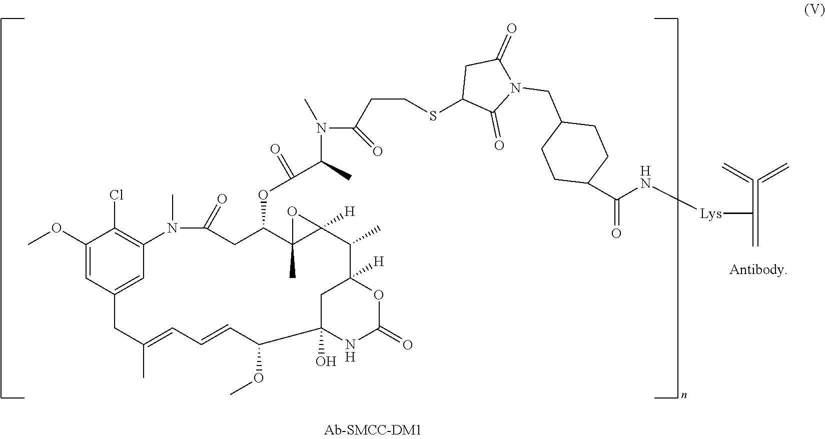

In an embodiment the conjugate is a conjugate of formula (III), (IV) or (V) as defined above, in which the antibody is an antibody described herein.

In formulas (III), (IV) and (V) above, “n” corresponds to the number of molecules of chemotherapeutic agent conjugated per molecule of antibody. It corresponds to the “drug-to-antibody ratio” (or “DAR”) defined below and may range from 1 to 10.

In an embodiment, the conjugate is tusamitamab ravtansine (CAS Registry No. 225408660-5).

In general, the conjugate can be obtained by a process comprising the steps of:
(i) bringing into contact an optionally-buffered aqueous solution of a an antibody according to the disclosure with solutions of a linker and a cytotoxic compound;
(ii) then optionally separating the conjugate which was formed in (i) from the unreacted antibody, linker and cytotoxic compounds.

The aqueous solution of cell-binding agent can be buffered with buffers such as, e.g. potassium phosphate, acetate, citrate or N-2-Hydroxyethylpiperazine-N′-2-ethanesulfonic acid (HEPES buffer). The buffer depends upon the nature of the cell-binding agent. The cytotoxic compound is in solution in an organic polar solvent, e.g. dimethyl sulfoxide (DMSO) or dimethylacetamide (DMA).

The reaction temperature is usually comprised between 2° and 40° C. The reaction time can vary from 1 to 24 hours. The reaction between the cell-binding agent and the cytotoxic agent can be monitored by size exclusion chromatography (SEC) with a refractometric and/or UV detector. If the conjugate yield is too low, the reaction time can be extended.

A number of different chromatography methods can be used by the person skilled in the art in order to perform the separation of step (ii): the conjugate can be purified e.g. by SEC, adsorption chromatography (such as ion exchange chromatography, IEC), hydrophobic interaction chromatography (HIC), affinity chromatography, mixed-support chromatography such as hydroxyapatite chromatography, or high performance liquid chromatography (HPLC). Purification by dialysis or diafiltration can also be used.

As used herein, the term “aggregates” means the associations which can be formed between two or more cell-binding agents, said agents being modified or not by conjugation. The aggregates can be formed under the influence of a great number of parameters, such as a high concentration of cell-binding agent in the solution, the pH of the solution, high shearing forces, the number of bonded dimers and their hydrophobic character, the temperature (see Wang & Gosh, 2008, J. Membrane Sci., 318: 311-316, and references cited therein); note that the relative influence of some of these parameters is not clearly established. In the case of proteins and antibodies, the person skilled in the art will refer to Cromwell et al. (2006, AAPS Journal, 8(3): E572-E579). The content in aggregates can be determined with techniques well known to the skilled person, such as SEC (see Walter et al., 1993, Anal. Biochem., 212(2): 469-480).

After step (i) or (ii), the conjugate-containing solution can be submitted to an additional step (iii) of chromatography, ultrafiltration and/or diafiltration.

The conjugate is recovered at the end of these steps in an aqueous solution.

According to an embodiment, the conjugate according to the disclosure is characterised by a “drug-to-antibody ratio” (or “DAR”) ranging from 1 to 10, for instance from 2 to 5, for example from 3 to 4. This is generally the case of conjugates including maytansinoid molecules.

This DAR number can vary with the nature of the antibody and of the drug (i.e. the growth-inhibitory agent) used along with the experimental conditions used for the conjugation (like the ratio growth-inhibitory agent/antibody, the reaction time, the nature of the solvent and of the cosolvent if any). Thus, the contact between the antibody and the growth-inhibitory agent leads to a mixture comprising several conjugates differing from one another by different drug-to-antibody ratios; optionally the naked antibody; optionally aggregates. The DAR that is determined is thus a mean value.

A method which can be used to determine the DAR consists in measuring spectrophotometrically the ratio of the absorbance at of a solution of substantially purified conjugate at λ_{D} and 280 nm. 280 nm is a wavelength generally used for measuring protein concentration, such as antibody concentration. The wavelength λ_{D} is selected so as to allow discriminating the drug from the antibody, i.e. as readily known to the skilled person, λ_{D} is a wavelength at which the drug has a high absorbance and λ_{D} is sufficiently remote from 280 nm to avoid substantial overlap in the absorbance peaks of the drug and antibody. λ_{D} may be selected as being 252 nm in the case of maytansinoid molecules. A method of DAR calculation may be derived from Antony S. Dimitrov (ed), LLC, 2009, Therapeutic Antibodies and Protocols, vol 525, 445, Springer Science:

The absorbances for the conjugate at λ_{D} (A_{λD}) and at 280 nm (A₂₈₀) are measured either on the monomeric peak of the size exclusion chromatography (SEC) analysis (allowing to calculate the “DAR(SEC)” parameter) or using a classic spectrophotometer apparatus (allowing to calculate the “DAR(UV)” parameter). The absorbances can be expressed as follows:

$$\begin{matrix}{A}_{λ D} = ( {c}_{D} \times {ε}_{D λ D} ) + ( {c}_{A} \times {ε}_{A λ D} ) \\ {A}_{280} = ( {c}_{D} \times {ε}_{D 280} ) + ( {c}_{A} \times {ε}_{A 280} )\end{matrix}$$

wherein:

c_{D} and c_{A} are respectively the concentrations in the solution of the drug and of the antibody,

ε_{DλD} and ε_{D280} are respectively the molar extinction coefficients of the drug at λ_{D} and 280 nm, and

ε_{AλD} and ε_{D280} are respectively the molar extinction coefficients of the antibody at λD and 280 nm.

Resolution of these two equations with two unknowns leads to the following equations:

$$\begin{matrix}{c}_{D} = [ ( {ε}_{A 280} \times {A}_{λ D} ) - ( {ε}_{A λ D} \times {A}_{280} ) ] / [ ( {ε}_{D λ D} \times {ε}_{A 280} ) - ( {ε}_{A λ D} \times {ε}_{D 280} ) ] \\ {c}_{A} = [ {A}_{280} - ( {c}_{D} \times {ε}_{D 280} ) ] / {ε}_{A 280}\end{matrix}$$

The average DAR is then calculated from the ratio of the drug concentration to that of the antibody: DAR=c_{D}/c_{A}.

### Tusamitamab Ravtansine

Tusamitamab ravtansine (CAS Registry No. 2254086-60-5) is an immunoconjugate ADC combining a humanized anti-CEACAM5 antibody (tusamitamab) and the maytansinoid derivative 4 (DM4) [N2-deacetyl-N2-(4-methyl-4-mercapto-1-oxopentyl)-maytansine], a potent antimitotic agent that inhibits microtubule assembly. DM4 is covalently bound to the antibody through an optimized linker SPDB [N-succinimidyl 4-(2-pyridyldithio)-butyrate] that is stable in plasma and cleavable inside cells. After binding and internalization in targeted cancer cells, the ADC is degraded, releasing cytotoxic DM4 metabolites.

The ADC tusamitamab ravtansine specifically binds to the A3B3 domain of human CEACAM5 and does not recognize other CEACAMs presenting A or/and B domains in their structure (CEACAM1, CEACAM6, CEACAM7 and CEACAM8). The naked antibody and the ADC bind to recombinant human CEACAM5 with an affinity of ˜0.02 nM (ELISA) and display high affinity for CEACAM5 expressing tumor cells (K_{D}^{APP} 0.24-0.68 nM). Preliminary experiments indicate that tusamitamab ravtansine is devoid of effector activity.

After binding to the CEACAM5 antigen, tusamitamab ravtansine is internalized by the cancer cells via antigen-mediated endocytosis, delivered to lysosomes and degraded into the lysine-linked derivative lysine-SPDB-DM4. The lysine-SPDB-DM4 gets further degraded in DM4 that is subsequently S-methylated to form methyl-DM4 [Me-DM4]; all three metabolites have potent cytotoxic activity through binding to tubulin and inhibition of microtubule polymerization.

### Pharmaceutical Compositions

The antibodies or immunoconjugates of the disclosure may be combined with pharmaceutically acceptable excipients, and optionally sustained-release matrices, such as biodegradable polymers, to form therapeutic compositions.

Thus, another object of the disclosure relates to a pharmaceutical composition comprising an antibody or an immunoconjugate of the disclosure and a pharmaceutically acceptable carrier or excipient.

The disclosure also relates to an antibody or an immunoconjugate according to the disclosure, for use as a medicament.

The disclosure also relates to an antibody, an immunoconjugate or a compound according to the disclosure, for use as for treating cancer.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

As used herein, “pharmaceutically-acceptable carriers” includes any and all solvents, dispersion media, coatings, antibacterial and antifungal agents, and the like that are physiologically compatible. Examples of suitable carriers, diluents and/or excipients include one or more of water, amino acids, saline, phosphate buffered saline, buffer phosphate, acetate, citrate, succinate; amino acids and derivates such as histidine, arginine, glycine, proline, glycylglycine; inorganic salts NaCl, calcium chloride; sugars or polyalcohols such as dextrose, glycerol, ethanol, sucrose, trehalose, mannitol; surfactants such as Polysorbate 80, polysorbate 20, poloxamer 188; and the like, as well as combination thereof. In many cases, it will be preferable to include isotonic agents, such as sugars, polyalcohols, or sodium chloride in the composition, and formulation may also contain an antioxidant such as tryptamine and a stabilizing agent such as Tween 20.

The form of the pharmaceutical compositions, the route of administration, the dosage and the regimen naturally depend upon the condition to be treated, the severity of the illness, the age, weight, and gender of the patient, etc.

The pharmaceutical compositions of the disclosure can be formulated for a topical, oral, parenteral, intranasal, intravenous, intramuscular, subcutaneous or intraocular administration and the like.

In an embodiment, the pharmaceutical compositions contain vehicles which are pharmaceutically acceptable for a formulation capable of being injected. These may be isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical composition can be administrated through drug combination devices.

The doses used for the administration can be adapted as a function of various parameters, and for instance as a function of the mode of administration used, of the relevant pathology, or alternatively of the desired duration of treatment.

To prepare pharmaceutical compositions, an effective amount of the antibody or immunoconjugate of the disclosure may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and injectable with the appropriate device or system for delivery without degradation. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant. Dispersions can also be prepared in glycerol, liquid polyethylene glycols, and mixtures thereof and in oils. Under ordinary conditions of storage and use, these preparations contain a preservative to prevent the growth of microorganisms.

A polypeptide, antibody or immunoconjugate of the disclosure can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, glycine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with any of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more concentrated, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The antibody or immunoconjugate of the disclosure may be formulated within a therapeutic mixture to comprise about 0.01 to 100 milligrams, per dose or so.

In addition to the antibody or immunoconjugate formulated for parenteral administration, such as intravenous or intramuscular injection, other pharmaceutically acceptable forms include, e.g. tablets or other solids for oral administration; time release capsules; and any other form currently used.

In certain embodiments, the use of liposomes and/or nanoparticles is contemplated for the introduction of polypeptides into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles, or biodegradable polylactide or polylactide co glycolide nanoparticules that meet these requirements are contemplated for use in the present disclosure, and such particles may be are easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 A, containing an aqueous solution in the core. The physical characteristics of liposomes depend on pH, ionic strength and the presence of divalent cations.

### Methods of Administration and Formulations

The methods described herein comprise administering a therapeutically effective amount of an anti-CEACAM5 ADC to a subject. As used herein, an “effective amount” or “therapeutically effective amount” is a dose of the therapeutic that results in treatment of CEACAM5 expressing cancer (e.g. lung cancer, gastric cancer, gastroesopageal junction cancer or esophageal cancer). As used herein, “treating” refers to causing a detectable improvement in one or more symptoms associated with CEACAM5 expressing cancer (e.g. lung cancer) or causing a biological effect (e.g., a decrease in the level of a particular biomarker) that is correlated with the underlying pathologic mechanism(s) giving rise to the condition or symptom(s). For example, a dose of anti-CEACAM5 ADC which causes an improvement in any of the following symptoms or conditions associated with CEACAM5 expressing cancer is deemed a “therapeutically effective amount”:

In another example, a treatment has not been effective when a dose of anti-CEACAM5 ADC does not result in a detectable improvement in one or more parameters or symptoms associated with a CEACAM5 expressing cancer (e.g., lung cancer, gastric cancer, gastroesophageal junction cancer or esophageal cancer) or which does not cause a biological effect that is correlated with the underlying pathologic mechanism(s) giving rise to the condition or symptom(s) of cancer.

According to some of these embodiments, the anti-CEACAM5 ADC is administered intravenously.

In accordance with the methods of the present disclosure, a therapeutically effective amount of anti-CEACAM5 ADC that is administered to the subject will vary depending upon the age and the size (e.g., body weight or body surface area) of the subject as well as the route of administration and other factors well known to those of ordinary skill in the art.

In certain embodiments, the dose of the ADC varies depending on the body surface area of the subject. In certain embodiments, the dose of anti-CEACAM5 ADC administered to the subject is from about 1 mg/m² to about 500 mg/m². In some embodiments, the dose of the ADC administered to the subject is from about 5 mg/m² to about 300 mg/m². In various embodiments, the dose of the ADC administered to the subject is from about 5 mg/m² to about 250 mg/m². In various embodiments, the dose of the ADC administered to the subject is from about 60 mg/m² to about 190 mg/m². In various embodiments, the dose is about 5, 10, 20, 30, 40, 50, 60, 70, 80, 90, 100, 110, 120, 130, 140, 150, 160, 170, 180, 190, 200, or 210 mg/m² based on the body surface area of the subject. In certain embodiments, the dose of the ADC is about 100 mg/m². In certain embodiments, the dose of the ADC is about 150 mg/m². In certain embodiments, the dose of the ADC is about 170 mg/m². In certain embodiments, the dose of the ADC is about 190 mg/m².

In various embodiments, the dose of the ADC is 5, 10, 20, 30, 40, 50, 60, 70, 80, 90, 100, 110, 120, 130, 140, 150, 160, 170, 180, 190, 200, or 210 mg/m² based on the body surface area of the subject. In certain embodiments, the dose of the ADC is 100 mg/m². In certain embodiments, the dose of the ADC is 150 mg/m². In certain embodiments, the dose of the ADC is 170 mg/m². In certain embodiments, the dose of the ADC is 190 mg/m².

An aspect of the disclosure is a method of treating a cancer in a subject, comprising
measuring circulating carcinoembryonic antigen (CEA) in a subject in need of treatment of cancer; and
administering to the subject an effective amount of an antibody-drug conjugate (ADC) comprising an anti-CEACAM5 antibody if the circulating CEA in the subject is about ≥5 ng/mL,

thereby treating the cancer.

An aspect of the disclosure is a circulating carcinoembryonic antigen (CEA) for use as a biomarker in a method for treating a cancer in a subject, the method comprising

measuring circulating carcinoembryonic antigen (CEA) in a subject in need of treatment of cancer; and

administering to the subject an effective amount of an antibody-drug conjugate (ADC) comprising an anti-CEACAM5 antibody if the circulating CEA in the subject is about ≥5 ng/mL,

thereby treating the cancer.

An aspect of the disclosure is a method of diagnosing a cancer in a subject, the method comprising at least the steps of:
a) measuring in a biological sample an amount of circulating carcinoembryonic antigen (CEA) in said subject; and
b) comparing the measured amount obtained at step a) with a value of reference, said value of reference being 5 ng/mL,

a measured amount obtained at step a) about ≥5 ng/mL being indicative of a cancer.

Circulating CEA can be measured using any suitable method, e.g., enzyme-linked immunoassay (ELISA), in a sample selected from serum, plasma, and whole blood. In certain embodiments, circulating CEA is measured using ELISA in a serum sample. In certain embodiments, circulating CEA is measured using ELISA in a plasma sample.

The circulating CEA can be measured at any time before, during, or after treatment. In certain embodiments, the circulating CEA is measured after performing immunohistochemistry analysis of CEACAM5 expression on tumor cells of the subject. In certain embodiments, the circulating CEA is first measured after performing immunohistochemistry analysis of CEACAM5 expression on tumor cells of the subject.

In certain embodiments, the cancer is selected from the group consisting of colorectal cancer, gastric cancer, gastroesophageal junction cancer, esophageal cancer, lung cancer, uterine cervix cancer, pancreatic cancer, ovarian cancer, thyroid cancer, bladder cancer, endometrial cancer, breast cancer, liver cancer, biliary tract cancer (e.g., cholangiocarcinoma), prostate cancer, and skin cancer.

In certain embodiments, the cancer is advanced or metastatic.

In certain embodiments, the cancer is gastric cancer.

In certain embodiments, the cancer is gastroesophageal junction cancer.

In certain embodiments, the cancer is esophageal cancer.

In certain embodiments, the cancer is lung cancer.

In certain embodiments, the cancer is small cell lung cancer (SCLC).

In certain embodiments, the cancer is non-small cell lung cancer (NSCLC).

In certain embodiments, the cancer is non-squamous non-small cell lung cancer (NSQ NSCLC).

In certain embodiments, the NSQ NSCLC is advanced or metastatic.

In certain embodiments, the subject has negative CEACAM5 expression on tumor cells (≥2+ intensity in <1% of cells) as measured by immunohistochemistry.

In certain embodiments, the subject has moderate CEACAM5 expression on tumor cells (≥2+ intensity in ≥1% and <50% of cells) as measured by immunohistochemistry.

In certain embodiments, the subject has high CEACAM5 expression on tumor cells (≥2+ intensity in ≥50% of cells) as measured by immunohistochemistry.

In certain embodiments, prior to treatment the circulating CEA in the subject is about ≥5 ng/mL. In certain embodiments, prior to treatment the circulating CEA in the subject is about ≥10 ng/mL. In certain embodiments, prior to treatment the circulating CEA in the subject is about ≥15 ng/mL. In certain embodiments, prior to treatment the circulating CEA in the subject is about ≥20 ng/mL. In certain embodiments, prior to treatment the circulating CEA in the subject is about ≥25 ng/mL. In certain embodiments, prior to treatment the circulating CEA in the subject is about ≥30 ng/mL. In certain embodiments, prior to treatment the circulating CEA in the subject is about ≥35 ng/mL. In certain embodiments, prior to treatment the circulating CEA in the subject is about ≥40 ng/mL. In certain embodiments, prior to treatment the circulating CEA in the subject is about ≥45 ng/mL. In certain embodiments, prior to treatment the circulating CEA in the subject is about ≥50 ng/mL. In certain embodiments, prior to treatment the circulating CEA in the subject is about ≥55 ng/mL. In certain embodiments, prior to treatment the circulating CEA in the subject is about ≥60 ng/mL. In certain embodiments, prior to treatment the circulating CEA in the subject is about ≥61 ng/mL. In certain embodiments, prior to treatment the circulating CEA in the subject is about ≥62 ng/mL. In certain embodiments, prior to treatment the circulating CEA in the subject is about ≥63 ng/mL. In certain embodiments, prior to treatment the circulating CEA in the subject is about ≥64 ng/mL. In certain embodiments, prior to treatment the circulating CEA in the subject is about ≥65 ng/mL. In certain embodiments, prior to treatment the circulating CEA in the subject is about ≥66 ng/mL. In certain embodiments, prior to treatment the circulating CEA in the subject is about ≥67 ng/mL. In certain embodiments, prior to treatment the circulating CEA in the subject is about ≥68 ng/mL. In certain embodiments, prior to treatment the circulating CEA in the subject is about ≥69 ng/mL. In certain embodiments, prior to treatment the circulating CEA in the subject is about ≥70 ng/mL. In certain embodiments, prior to treatment the circulating CEA in the subject is about ≥71 ng/mL. In certain embodiments, prior to treatment the circulating CEA in the subject is about ≥72 ng/mL. In certain embodiments, prior to treatment the circulating CEA in the subject is about ≥73 ng/mL. In certain embodiments, prior to treatment the circulating CEA in the subject is about ≥74 ng/mL. In certain embodiments, prior to treatment the circulating CEA in the subject is about ≥75 ng/mL. In certain embodiments, prior to treatment the circulating CEA in the subject is about ≥76 ng/mL. In certain embodiments, prior to treatment the circulating CEA in the subject is about ≥77 ng/mL. In certain embodiments, prior to treatment the circulating CEA in the subject is about ≥78 ng/mL. In certain embodiments, prior to treatment the circulating CEA in the subject is about ≥79 ng/mL. In certain embodiments, prior to treatment the circulating CEA in the subject is about ≥80 ng/mL. In certain embodiments, prior to treatment the circulating CEA in the subject is about ≥85 ng/mL. In certain embodiments, prior to treatment the circulating CEA in the subject is about ≥90 ng/mL. In certain embodiments, prior to treatment the circulating CEA in the subject is about ≥95 ng/mL. In certain embodiments, prior to treatment the circulating CEA is about ≥100 ng/mL.

In an embodiment, the anti-CEACAM5 antibody is tusamitamab.

In certain embodiments, the ADC is tusamitamab ravtansine.

In certain embodiments, the tusamitamab ravtansine is administered in a dose of about 60 mg/m² to about ≥190 mg/m². In certain embodiments, the tusamitamab ravtansine is administered in a dose of 60 mg/m² to 190 mg/m².

In certain embodiments, the tusamitamab ravtansine is administered in a dose of about 60 mg/m². In certain embodiments, the tusamitamab ravtansine is administered in a dose of about 70 mg/m². In certain embodiments, the tusamitamab ravtansine is administered in a dose of about 80 mg/m². In certain embodiments, the tusamitamab ravtansine is administered in a dose of about 90 mg/m². In certain embodiments, the tusamitamab ravtansine is administered in a dose of about 100 mg/m². In certain embodiments, the tusamitamab ravtansine is administered in a dose of about 110 mg/m². In certain embodiments, the tusamitamab ravtansine is administered in a dose of about 120 mg/m². In certain embodiments, the tusamitamab ravtansine is administered in a dose of about 130 mg/m². In certain embodiments, the tusamitamab ravtansine is administered in a dose of about 140 mg/m². In certain embodiments, the tusamitamab ravtansine is administered in a dose of about 150 mg/m². In certain embodiments, the tusamitamab ravtansine is administered in a dose of about 160 mg/m². In certain embodiments, the tusamitamab ravtansine is administered in a dose of about 170 mg/m². In certain embodiments, the tusamitamab ravtansine is administered in a dose of about 180 mg/m². In certain embodiments, the tusamitamab ravtansine is administered in a dose of about 190 mg/m². In certain embodiments, the tusamitamab ravtansine is administered in a dose of about 200 mg/m²

In certain embodiments, the tusamitamab ravtansine is administered in a dose of 90 mg/m². In certain embodiments, the tusamitamab ravtansine is administered in a dose of 100 mg/m². In certain embodiments, the tusamitamab ravtansine is administered in a dose of 110 mg/m². In certain embodiments, the tusamitamab ravtansine is administered in a dose of 120 mg/m². In certain embodiments, the tusamitamab ravtansine is administered in a dose of 130 mg/m². In certain embodiments, the tusamitamab ravtansine is administered in a dose of 140 mg/m². In certain embodiments, the tusamitamab ravtansine is administered in a dose of 150 mg/m². In certain embodiments, the tusamitamab ravtansine is administered in a dose of 160 mg/m². In certain embodiments, the tusamitamab ravtansine is administered in a dose of 170 mg/m². In certain embodiments, the tusamitamab ravtansine is administered in a dose of 180 mg/m². In certain embodiments, the tusamitamab ravtansine is administered in a dose of 190 mg/m². In certain embodiments, the tusamitamab ravtansine is administered in a dose of 200 mg/m².

In certain embodiments, the tusamitamab ravtansine is administered about once every two weeks. In certain embodiments, the tusamitamab ravtansine is administered about once every three weeks. In certain embodiments, the tusamitamab ravtansine is administered about once every four weeks. In certain embodiments, the tusamitamab ravtansine is administered about once every five weeks. In certain embodiments, the tusamitamab ravtansine is administered about once every six weeks.

In certain embodiments, the tusamitamab ravtansine is administered once every two weeks. In certain embodiments, the tusamitamab ravtansine is administered once every three weeks. In certain embodiments, the tusamitamab ravtansine is administered once every four weeks. In certain embodiments, the tusamitamab ravtansine is administered once every five weeks. In certain embodiments, the tusamitamab ravtansine is administered once every six weeks.

In certain embodiments, the tusamitamab ravtansine is administered in a dose of about 100-200 mg/m² about once every two weeks. In certain embodiments, the tusamitamab ravtansine is administered in a dose of about 100-210 mg/m² once every two weeks.

In certain embodiments, the tusamitamab ravtansine is administered in a dose of 100-200 mg/m² about once every two weeks. In certain embodiments, the tusamitamab ravtansine is administered in a dose of 100-200 mg/m² once every two weeks.

In certain embodiments, the tusamitamab ravtansine is administered in a dose of about 100 mg/m² about once every two weeks. In certain embodiments, the tusamitamab ravtansine is administered in a dose of about 100 mg/m² once every two weeks.

In certain embodiments, the tusamitamab ravtansine is administered in a dose of 100 mg/m² about once every two weeks. In certain embodiments, the tusamitamab ravtansine is administered in a dose of 100 mg/m² once every two weeks.

In certain embodiments, the tusamitamab ravtansine is administered in a dose of about 100-200 mg/m² about once every three weeks. In certain embodiments, the tusamitamab ravtansine is administered in a dose of about 100-210 mg/m² once every three weeks.

In certain embodiments, the tusamitamab ravtansine is administered in a dose of 100-200 mg/m² about once every three weeks. In certain embodiments, the tusamitamab ravtansine is administered in a dose of 100-200 mg/m² once every three weeks.

In certain embodiments, the tusamitamab ravtansine is administered in a dose of about 100 mg/m² about once every three weeks. In certain embodiments, the tusamitamab ravtansine is administered in a dose of about 100 mg/m² once every three weeks.

In certain embodiments, the tusamitamab ravtansine is administered in a dose of 100 mg/m² about once every three weeks. In certain embodiments, the tusamitamab ravtansine is administered in a dose of 100 mg/m² once every three weeks.

In certain embodiments, the tusamitamab ravtansine is administered in a dose of about 100-200 mg/m² about once every six weeks. In certain embodiments, the tusamitamab ravtansine is administered in a dose of about 100-200 mg/m² once every six weeks.

In certain embodiments, the tusamitamab ravtansine is administered in a dose of 100-200 mg/m² about once every six weeks. In certain embodiments, the tusamitamab ravtansine is administered in a dose of 100-200 mg/m² once every six weeks.

In certain embodiments, the tusamitamab ravtansine is administered in a dose of about 100 mg/m² about once every six weeks. In certain embodiments, the tusamitamab ravtansine is administered in a dose of about 100 mg/m² once every six weeks.

In certain embodiments, the tusamitamab ravtansine is administered in a dose of 100 mg/m² about once every six weeks. In certain embodiments, the tusamitamab ravtansine is administered in a dose of 100 mg/m² once every six weeks.

In certain embodiments, the method further comprises administering to the subject an effective amount of at least one additional agent effective to treat the cancer.

In certain embodiments, the additional agent is selected from the group consisting of an immune checkpoint inhibitor (ICI), a platinum-based chemotherapy (e.g., cisplatin or carboplatin), pemetrexed, anti-VEGFR2, FOLFOX, FOLFIRI, TAS-102, anti-EGFR, and any combination thereof.

In certain embodiments, the ICI is an anti-PD-1 antibody.

In certain embodiments, the anti-PD-1 antibody is selected from the group consisting of pembrolizumab, nivolumab, cemiplimab, sintilimab, dostarlimab, and tislelizumab.

In certain embodiments, the anti-PD-1 antibody is pembrolizumab.

In certain embodiments, the ICI is an anti-PD-L1 antibody.

In certain embodiments, the anti-PD-L1 antibody is selected from the group consisting of atezolizumab, avelumab, durvalumab, envafolimab, BMS-936559, CK-301, CS-1001, SHR-1316 (HTI-1088), CBT-502 (TQB-2450), and any combination thereof.

In certain embodiments, the anti-PD-L1 antibody is selected from the group consisting of atezolizumab, avelumab, and durvalumab.

In certain embodiments, the method comprises administering to the subject an effective amount of tusamitamab ravtansine and pembrolizumab.

In certain embodiments, the method further comprises administering to the subject an effective amount of a platinum-based chemotherapy.

In certain embodiments, the platinum-based chemotherapy is selected from cisplatin and carboplatin.

In certain embodiments, the method further comprises administering to the subject an effective amount of pemetrexed.

In certain embodiments, the method comprises administering to the subject an effective amount of tusamitamab ravtansine, pembrolizumab, and cisplatin.

In certain embodiments, the method comprises administering to the subject an effective amount of tusamitamab ravtansine, pembrolizumab, cisplatin, and pemetrexed.

In certain embodiments, the method comprises administering to the subject an effective amount of tusamitamab ravtansine, pembrolizumab, and carboplatin.

In certain embodiments, the method comprises administering to the subject an effective amount of tusamitamab ravtansine, pembrolizumab, carboplatin, and pemetrexed.

In certain embodiments, the additional agent is an anti-VEGFR2. In an embodiment, the anti-VEGFR2 is ramucirumab (CYRAMZA®).

In certain embodiments, the additional agent is FOLFOX. FOLFOX is a chemotherapy regimen comprising folinic acid (leucovorin), fluorouracil (5-FU), and oxaliplatin.

In certain embodiments, the additional agent is FOLFIRI. FOLFIRI is a chemotherapy regimen comprising folinic acid (leucovorin), fluorouracil (5-FU), and irinotecan (ONIVYDE®).

In certain embodiments, the additional agent is TAS-102. TAS-102 is a combination of trifluridine and tipiracil hydrochloride.

In certain embodiments, the additional agent is an anti-EGFR. In certain embodiments, 25 the anti-EGFR is selected from cetuximab (ERBITUX®) and necitumumab (PORTRAZZA®).

In certain embodiments, the subject has moderate CEACAM5 expression on tumor cells (≥2+ intensity in ≥1% and <50% of cells) as measured by immunohistochemistry, and circulating CEA about ≥100 ng/mL, and the ADC is tusamitamab ravtansine.

In certain embodiments, the subject has advanced or metastatic cancer, moderate CEACAM5 expression on tumor cells (≥2+ intensity in ≥1% and <50% of cells) as measured by immunohistochemistry, and circulating CEA about ≥100 ng/mL, and the ADC is tusamitamab ravtansine.

In certain embodiments, the subject has negative or low CEACAM5 expression on tumor cells (≥2+ intensity in <1% of cells) as measured by immunohistochemistry, and circulating CEA about ≥100 ng/mL, and the ADC is tusamitamab ravtansine.

In certain embodiments, the subject has advanced or metastatic cancer, negative or low CEACAM5 expression on tumor cells (≥2+ intensity in <1% of cells) as measured by immunohistochemistry, and circulating CEA about ≥100 ng/mL, and the ADC is tusamitamab ravtansine.

In certain embodiments, the subject has NSQ NSCLC, moderate CEACAM5 expression on tumor cells (≥2+ intensity in ≥1% and <50% of cells) as measured by immunohistochemistry, and circulating CEA about ≥100 ng/mL, and the ADC is tusamitamab ravtansine.

In certain embodiments, the subject has advanced or metastatic NSQ NSCLC, moderate CEACAM5 expression on tumor cells (≥2+ intensity in >1% and <50% of cells) as measured by immunohistochemistry, and circulating CEA about ≥100 ng/mL, and the ADC is tusamitamab ravtansine.

In certain embodiments, the subject has NSQ NSCLC, negative or low CEACAM5 expression on tumor cells (≥2+ intensity in <1% of cells) as measured by immunohistochemistry, and circulating CEA about ≥100 ng/mL, and the ADC is tusamitamab ravtansine.

In certain embodiments, the subject has advanced or metastatic NSQ NSCLC, negative or low CEACAM5 expression on tumor cells (≥2+ intensity in <1% of cells) as measured by immunohistochemistry, and circulating CEA about ≥100 ng/mL, and the ADC is tusamitamab ravtansine.

In certain embodiments, the subject has advanced or metastatic NSQ NSCLC, moderate CEACAM5 expression on tumor cells (≥2+ intensity in ≥1% and <50% of cells) as measured by immunohistochemistry, and circulating CEA about ≥100 ng/mL, and the ADC is tusamitamab ravtansine.

An aspect of the disclosure is a method of treating a cancer in a subject, comprising
selecting a subject having cancer with a circulating carcinoembryonic antigen (CEA) about ≥5 ng/mL; and
administering to the subject an effective amount of an antibody-drug conjugate (ADC) comprising an anti-CEACAM5 antibody,

thereby treating the cancer.

In an embodiment, the anti-CEACAM5 antibody is tusamitamab.

Another aspect of the disclosure is a circulating carcinoembryonic antigen (CEA) for use as a biomarker in a method of treating a cancer in a subject, the method comprising

selecting a subject having cancer with a circulating carcinoembryonic antigen (CEA) about ≥5 ng/mL; and

administering to the subject an effective amount of an antibody-drug conjugate (ADC) comprising an anti-CEACAM5 antibody,

thereby treating the cancer.

Circulating CEA can be measured using any suitable method, e.g., enzyme-linked immunoassay (ELISA), in a sample selected from serum, plasma, and whole blood. In certain embodiments, circulating CEA is measured using ELISA in a serum sample. In certain embodiments, circulating CEA is measured using ELISA in a plasma sample.

The circulating CEA can be measured at any time before, during, or after treatment. In certain embodiments, the circulating CEA is measured after performing immunohistochemistry analysis of CEACAM5 expression on tumor cells of the subject. In certain embodiments, the circulating CEA is first measured after performing immunohistochemistry analysis of CEACAM5 expression on tumor cells of the subject.

In certain embodiments, the cancer is selected from the group consisting of colorectal cancer, gastric cancer, gastroesophageal junction cancer, esophageal cancer, lung cancer, uterine cervix cancer, pancreatic cancer, ovarian cancer, thyroid cancer, bladder cancer, endometrial cancer, breast cancer, liver cancer, biliary tract cancer (e.g., cholangiocarcinoma), prostate cancer, and skin cancer.

In certain embodiments, the cancer is advanced or metastatic.

In certain embodiments, the cancer is gastric cancer.

In certain embodiments, the cancer is lung cancer.

In certain embodiments, the cancer is small cell lung cancer (SCLC).

In certain embodiments, the cancer is non-small cell lung cancer (NSCLC).

In certain embodiments, the cancer is non-squamous non-small cell lung cancer (NSQ NSCLC).

In certain embodiments, the NSQ NSCLC is advanced or metastatic.

In certain embodiments, the subject has negative CEACAM5 expression on tumor cells (≥2+ intensity in <1% of cells) as measured by immunohistochemistry.

In certain embodiments, the subject has moderate CEACAM5 expression on tumor cells (≥2+ intensity in ≥1% and <50% of cells) as measured by immunohistochemistry.

In certain embodiments, the subject has high CEACAM5 expression on tumor cells (≥2+ intensity in ≥50% of cells) as measured by immunohistochemistry.

In certain embodiments, prior to treatment the circulating CEA in the subject is about ≥5 ng/mL. In certain embodiments, prior to treatment the circulating CEA in the subject is about ≥10 ng/mL. In certain embodiments, prior to treatment the circulating CEA in the subject is about ≥15 ng/mL. In certain embodiments, prior to treatment the circulating CEA in the subject is about ≥20 ng/mL. In certain embodiments, prior to treatment the circulating CEA in the subject is about ≥25 ng/mL. In certain embodiments, prior to treatment the circulating CEA in the subject is about ≥30 ng/mL. In certain embodiments, prior to treatment the circulating CEA in the subject is about ≥35 ng/mL. In certain embodiments, prior to treatment the circulating CEA in the subject is about ≥40 ng/mL. In certain embodiments, prior to treatment the circulating CEA in the subject is about ≥45 ng/mL. In certain embodiments, prior to treatment the circulating CEA in the subject is about ≥50 ng/mL. In certain embodiments, prior to treatment the circulating CEA in the subject is about ≥55 ng/mL. In certain embodiments, prior to treatment the circulating CEA in the subject is about ≥60 ng/mL. In certain embodiments, prior to treatment the circulating CEA in the subject is about ≥61 ng/mL. In certain embodiments, prior to treatment the circulating CEA in the subject is about ≥62 ng/mL. In certain embodiments, prior to treatment the circulating CEA in the subject is about ≥63 ng/mL. In certain embodiments, prior to treatment the circulating CEA in the subject is about ≥64 ng/mL. In certain embodiments, prior to treatment the circulating CEA in the subject is about ≥65 ng/mL. In certain embodiments, prior to treatment the circulating CEA in the subject is about ≥66 ng/mL. In certain embodiments, prior to treatment the circulating CEA in the subject is about ≥67 ng/mL. In certain embodiments, prior to treatment the circulating CEA in the subject is about ≥68 ng/mL. In certain embodiments, prior to treatment the circulating CEA in the subject is about ≥69 ng/mL. In certain embodiments, prior to treatment the circulating CEA in the subject is about ≥70 ng/mL. In certain embodiments, prior to treatment the circulating CEA in the subject is about ≥71 ng/mL. In certain embodiments, prior to treatment the circulating CEA in the subject is about ≥72 ng/mL. In certain embodiments, prior to treatment the circulating CEA in the subject is about ≥73 ng/mL. In certain embodiments, prior to treatment the circulating CEA in the subject is about ≥74 ng/mL. In certain embodiments, prior to treatment the circulating CEA in the subject is about ≥75 ng/mL. In certain embodiments, prior to treatment the circulating CEA in the subject is about ≥76 ng/mL. In certain embodiments, prior to treatment the circulating CEA in the subject is about ≥77 ng/mL. In certain embodiments, prior to treatment the circulating CEA in the subject is about ≥78 ng/mL. In certain embodiments, prior to treatment the circulating CEA in the subject is about ≥79 ng/mL. In certain embodiments, prior to treatment the circulating CEA in the subject is about ≥80 ng/mL. In certain embodiments, prior to treatment the circulating CEA in the subject is about ≥85 ng/mL. In certain embodiments, prior to treatment the circulating CEA in the subject is about ≥90 ng/mL. In certain embodiments, prior to treatment the circulating CEA in the subject is about ≥95 ng/mL. In certain embodiments, prior to treatment the circulating CEA is about ≥100 ng/mL.

In certain embodiments, the ADC is tusamitamab ravtansine.

In certain embodiments, the tusamitamab ravtansine is administered in a dose of about 60 mg/m² to about ≥190 mg/m². In certain embodiments, the tusamitamab ravtansine is administered in a dose of 60 mg/m² to 190 mg/m²

In certain embodiments, the tusamitamab ravtansine is administered in a dose of about 60 mg/m². In certain embodiments, the tusamitamab ravtansine is administered in a dose of about 70 mg/m². In certain embodiments, the tusamitamab ravtansine is administered in a dose of about 80 mg/m². In certain embodiments, the tusamitamab ravtansine is administered in a dose of about 90 mg/m². In certain embodiments, the tusamitamab ravtansine is administered in a dose of about 100 mg/m². In certain embodiments, the tusamitamab ravtansine is administered in a dose of about 110 mg/m². In certain embodiments, the tusamitamab ravtansine is administered in a dose of about 120 mg/m². In certain embodiments, the tusamitamab ravtansine is administered in a dose of about 130 mg/m². In certain embodiments, the tusamitamab ravtansine is administered in a dose of about 140 mg/m². In certain embodiments, the tusamitamab ravtansine is administered in a dose of about 150 mg/m². In certain embodiments, the tusamitamab ravtansine is administered in a dose of about 160 mg/m². In certain embodiments, the tusamitamab ravtansine is administered in a dose of about 170 mg/m². In certain embodiments, the tusamitamab ravtansine is administered in a dose of about 180 mg/m². In certain embodiments, the tusamitamab ravtansine is administered in a dose of about 190 mg/m². In certain embodiments, the tusamitamab ravtansine is administered in a dose of about 200 mg/m²

In certain embodiments, the tusamitamab ravtansine is administered in a dose of 90 mg/m². In certain embodiments, the tusamitamab ravtansine is administered in a dose of 100 mg/m². In certain embodiments, the tusamitamab ravtansine is administered in a dose of 110 mg/m². In certain embodiments, the tusamitamab ravtansine is administered in a dose of 120 mg/m². In certain embodiments, the tusamitamab ravtansine is administered in a dose of 130 mg/m². In certain embodiments, the tusamitamab ravtansine is administered in a dose of 140 mg/m². In certain embodiments, the tusamitamab ravtansine is administered in a dose of 150 mg/m². In certain embodiments, the tusamitamab ravtansine is administered in a dose of 160 mg/m². In certain embodiments, the tusamitamab ravtansine is administered in a dose of 170 mg/m². In certain embodiments, the tusamitamab ravtansine is administered in a dose of 180 mg/m². In certain embodiments, the tusamitamab ravtansine is administered in a dose of 190 mg/m². In certain embodiments, the tusamitamab ravtansine is administered in a dose of 200 mg/m².

In certain embodiments, the tusamitamab ravtansine is administered about once every two weeks. In certain embodiments, the tusamitamab ravtansine is administered about once every three weeks. In certain embodiments, the tusamitamab ravtansine is administered about once every four weeks. In certain embodiments, the tusamitamab ravtansine is administered about once every five weeks. In certain embodiments, the tusamitamab ravtansine is administered about once every six weeks.

In certain embodiments, the tusamitamab ravtansine is administered once every two weeks. In certain embodiments, the tusamitamab ravtansine is administered once every three weeks. In certain embodiments, the tusamitamab ravtansine is administered once every four weeks. In certain embodiments, the tusamitamab ravtansine is administered once every five weeks. In certain embodiments, the tusamitamab ravtansine is administered once every six weeks.

In certain embodiments, the tusamitamab ravtansine is administered in a dose of about 100-200 mg/m² about once every two weeks. In certain embodiments, the tusamitamab ravtansine is administered in a dose of about ≥100-200 mg/m² once every two weeks.

In certain embodiments, the tusamitamab ravtansine is administered in a dose of 100-200 mg/m² about once every two weeks. In certain embodiments, the tusamitamab ravtansine is administered in a dose of 100-200 mg/m² once every two weeks.

In certain embodiments, the tusamitamab ravtansine is administered in a dose of about 100 mg/m² about once every two weeks. In certain embodiments, the tusamitamab ravtansine is administered in a dose of about ≥100 mg/m² once every two weeks.

In certain embodiments, the tusamitamab ravtansine is administered in a dose of 100 mg/m² about once every two weeks. In certain embodiments, the tusamitamab ravtansine is administered in a dose of 100 mg/m² once every two weeks.

In certain embodiments, the tusamitamab ravtansine is administered in a dose of about 100-200 mg/m² about once every three weeks. In certain embodiments, the tusamitamab ravtansine is administered in a dose of about ≥100-200 mg/m² once every three weeks.

In certain embodiments, the tusamitamab ravtansine is administered in a dose of 100-200 mg/m² about once every three weeks. In certain embodiments, the tusamitamab ravtansine is administered in a dose of 100-200 mg/m² once every three weeks.

In certain embodiments, the tusamitamab ravtansine is administered in a dose of about 100 mg/m² about once every three weeks. In certain embodiments, the tusamitamab ravtansine is administered in a dose of about ≥100 mg/m² once every three weeks.

In certain embodiments, the tusamitamab ravtansine is administered in a dose of 100 mg/m² about once every three weeks. In certain embodiments, the tusamitamab ravtansine is administered in a dose of 100 mg/m² once every three weeks.

In certain embodiments, the tusamitamab ravtansine is administered in a dose of about 100-200 mg/m² about once every six weeks. In certain embodiments, the tusamitamab ravtansine is administered in a dose of about ≥100-200 mg/m² once every six weeks.

In certain embodiments, the tusamitamab ravtansine is administered in a dose of 100-200 mg/m² about once every six weeks. In certain embodiments, the tusamitamab ravtansine is administered in a dose of 100-200 mg/m² once every six weeks.

In certain embodiments, the tusamitamab ravtansine is administered in a dose of about 100 mg/m² about once every six weeks. In certain embodiments, the tusamitamab ravtansine is administered in a dose of about ≥100 mg/m² once every six weeks.

In certain embodiments, the tusamitamab ravtansine is administered in a dose of 100 mg/m² about once every six weeks. In certain embodiments, the tusamitamab ravtansine is administered in a dose of 100 mg/m² once every six weeks.

In certain embodiments, the tusamitamab ravtansine is administered in a dose of 100 mg/m² about once every three weeks. In certain embodiments, the tusamitamab ravtansine is administered in a dose of 100 mg/m² once every three weeks.

In certain embodiments, the tusamitamab ravtansine is administered in a dose of 100 mg/m² about once every six weeks. In certain embodiments, the tusamitamab ravtansine is administered in a dose of 100 mg/m² once every six weeks.

In certain embodiments, the method further comprises administering to the subject an effective amount of at least one additional agent effective to treat the cancer.

In certain embodiments, the additional agent is selected from the group consisting of an immune checkpoint inhibitor (ICI), a platinum-based chemotherapy (e.g., cisplatin or carboplatin), pemetrexed, anti-VEGFR2, FOLFOX, FOLFIRI, TAS-102, anti-EGFR, and any combination thereof.

In certain embodiments, the ICI is an anti-PD-1 antibody.

In certain embodiments, the anti-PD-1 antibody is selected from the group consisting of pembrolizumab, nivolumab, cemiplimab, sintilimab, dostarlimab, and tislelizumab.

In certain embodiments, the anti-PD-1 antibody is pembrolizumab.

In certain embodiments, the ICI is an anti-PD-L1 antibody.

In certain embodiments, the anti-PD-L1 antibody is selected from the group consisting of atezolizumab, avelumab, durvalumab, envafolimab, BMS-936559, CK-301, CS-1001, SHR-1316 (HTI-1088), CBT-502 (TQB-2450), and any combination thereof.

In certain embodiments, the anti-PD-L1 antibody is selected from the group consisting of atezolizumab, avelumab, and durvalumab.

In certain embodiments, the method comprises administering to the subject an effective amount of tusamitamab ravtansine and pembrolizumab.

In certain embodiments, the method further comprises administering to the subject an effective amount of a platinum-based chemotherapy.

In certain embodiments, the platinum-based chemotherapy is selected from cisplatin and carboplatin.

In certain embodiments, the method further comprises administering to the subject an effective amount of pemetrexed.

In certain embodiments, the method comprises administering to the subject an effective amount of tusamitamab ravtansine, pembrolizumab, and cisplatin.

In certain embodiments, the method comprises administering to the subject an effective amount of tusamitamab ravtansine, pembrolizumab, cisplatin, and pemetrexed.

In certain embodiments, the method comprises administering to the subject an effective amount of tusamitamab ravtansine, pembrolizumab, and carboplatin.

In certain embodiments, the method comprises administering to the subject an effective amount of tusamitamab ravtansine, pembrolizumab, carboplatin, and pemetrexed.

In certain embodiments, the additional agent is an anti-VEGFR2. In an embodiment, the anti-VEGFR2 is ramucirumab (CYRAMZA®).

In certain embodiments, the additional agent is FOLFOX. FOLFOX is a chemotherapy regimen comprising folinic acid (leucovorin), fluorouracil (5-FU), and oxaliplatin.

In certain embodiments, the additional agent is FOLFIRI. FOLFIRI is a chemotherapy regimen comprising folinic acid (leucovorin), fluorouracil (5-FU), and irinotecan (ONIVYDE®).

In certain embodiments, the additional agent is TAS-102. TAS-102 is a combination of trifluridine and tipiracil hydrochloride.

In certain embodiments, the additional agent is an anti-EGFR. In certain embodiments, the anti-EGFR is selected from cetuximab (ERBITUX®) and necitumumab (PORTRAZZA®).

In certain embodiments, the subject has moderate CEACAM5 expression on tumor cells (≥2+ intensity in ≥1% and <50% of cells) as measured by immunohistochemistry, and circulating CEA about ≥100 ng/mL, and the ADC is tusamitamab ravtansine.

In certain embodiments, the subject has advanced or metastatic cancer, moderate CEACAM5 expression on tumor cells (≥2+ intensity in ≥1% and <50% of cells) as measured by immunohistochemistry, and circulating CEA about ≥100 ng/mL, and the ADC is tusamitamab ravtansine.

In certain embodiments, the subject has negative or low CEACAM5 expression on tumor cells (≥2+ intensity in <1% of cells) as measured by immunohistochemistry, and circulating CEA about ≥100 ng/mL, and the ADC is tusamitamab ravtansine.

In certain embodiments, the subject has advanced or metastatic cancer, negative or low CEACAM5 expression on tumor cells (≥2+ intensity in <1% of cells) as measured by immunohistochemistry, and circulating CEA about ≥100 ng/mL, and the ADC is tusamitamab ravtansine.

In certain embodiments, the subject has NSQ NSCLC, moderate CEACAM5 expression on tumor cells (≥2+ intensity in ≥1% and <50% of cells) as measured by immunohistochemistry, and circulating CEA about ≥100 ng/mL, and the ADC is tusamitamab ravtansine.

In certain embodiments, the subject has advanced or metastatic NSQ NSCLC, moderate CEACAM5 expression on tumor cells (≥2+ intensity in ≥1% and <50% of cells) as measured by immunohistochemistry, and circulating CEA about ≥100 ng/mL, and the ADC is tusamitamab ravtansine.

In certain embodiments, the subject has NSQ NSCLC, negative or low CEACAM5 expression on tumor cells (≥2+ intensity in <1% of cells) as measured by immunohistochemistry, and circulating CEA about ≥100 ng/mL, and the ADC is tusamitamab ravtansine.

In certain embodiments, the subject has advanced or metastatic NSQ NSCLC, negative or negative or low CEACAM5 expression on tumor cells (≥2+ intensity in <1% of cells) as measured by immunohistochemistry, and circulating CEA about ≥100 ng/mL, and the ADC is tusamitamab ravtansine.

In certain embodiments, the subject has advanced or metastatic NSQ NSCLC, moderate CEACAM5 expression on tumor cells (≥2+ intensity in ≥1% and <50% of cells) as measured by immunohistochemistry, and circulating CEA about ≥100 ng/mL, and the ADC is tusamitamab ravtansine.

An aspect of the disclosure is a method of treating a cancer in a subject, comprising
measuring a first circulating carcinoembryonic antigen (CEA) in a subject in need of treatment of cancer;
administering to the subject a first effective amount of an antibody-drug conjugate (ADC) comprising an anti-CEACAM5 antibody if the first measured circulating CEA in the subject prior to the administering is about ≥5 ng/mL;
measuring a second circulating CEA in the subject after the administering step; and
administering to the subject a second effective amount of the ADC if the second measured circulating CEA in the subject is less than the first measured circulating CEA,

thereby treating the cancer.

Another aspect of the disclosure is a circulating carcinoembryonic antigen (CEA) for use as a biomarker in a method of treating a cancer in a subject, the method comprising

measuring a first circulating carcinoembryonic antigen (CEA) in a subject in need of treatment of cancer;

administering to the subject a first effective amount of an antibody-drug conjugate (ADC) comprising an anti-CEACAM5 antibody if the first measured circulating CEA in the subject prior to the administering is about ≥5 ng/mL;

measuring a second circulating CEA in the subject after the administering step; and

administering to the subject a second effective amount of the ADC if the second measured circulating CEA in the subject is less than the first measured circulating CEA,

thereby treating the cancer.

In an embodiment, the anti-CEACAM5 antibody is tusamitamab.

Circulating CEA can be measured using any suitable method, e.g., enzyme-linked immunoassay (ELISA), in a sample selected from serum, plasma, and whole blood. In certain embodiments, circulating CEA is measured using ELISA in a serum sample. In certain embodiments, circulating CEA is measured using ELISA in a plasma sample.

The circulating CEA can be measured at any time before, during, or after treatment. In certain embodiments, the circulating CEA is measured after performing immunohistochemistry analysis of CEACAM5 expression on tumor cells of the subject. In certain embodiments, the circulating CEA is first measured after performing immunohistochemistry analysis of CEACAM5 expression on tumor cells of the subject.

In certain embodiments, the cancer is selected from the group consisting of colorectal cancer, gastric cancer, gastroesophageal junction cancer, esophageal cancer, lung cancer, uterine cervix cancer, pancreatic cancer, ovarian cancer, thyroid cancer, bladder cancer, endometrial cancer, breast cancer, liver cancer, biliary tract cancer (e.g., cholangiocarcinoma), prostate cancer, and skin cancer.

In certain embodiments, the cancer is advanced or metastatic.

In certain embodiments, the cancer is gastric cancer.

In certain embodiments, the cancer is lung cancer.

In certain embodiments, the cancer is small cell lung cancer (SCLC).

In certain embodiments, the cancer is non-small cell lung cancer (NSCLC).

In certain embodiments, the cancer is non-squamous non-small cell lung cancer (NSQ NSCLC).

In certain embodiments, the NSQ NSCLC is advanced or metastatic.

In certain embodiments, the subject has negative CEACAM5 expression on tumor cells (≥2+ intensity in <1% of cells) as measured by immunohistochemistry.

In certain embodiments, the subject has moderate CEACAM5 expression on tumor cells (≥2+ intensity in ≥1% and <50% of cells) as measured by immunohistochemistry.

In certain embodiments, the subject has high CEACAM5 expression on tumor cells (2+ intensity in 50% of cells) as measured by immunohistochemistry.

In certain embodiments, prior to treatment the circulating CEA in the subject is about 5 ng/mL. In certain embodiments, prior to treatment the circulating CEA in the subject is about ≥10 ng/mL. In certain embodiments, prior to treatment the circulating CEA in the subject is about ≥15 ng/mL. In certain embodiments, prior to treatment the circulating CEA in the subject is about ≥20 ng/mL. In certain embodiments, prior to treatment the circulating CEA in the subject is about ≥25 ng/mL. In certain embodiments, prior to treatment the circulating CEA in the subject is about ≥30 ng/mL. In certain embodiments, prior to treatment the circulating CEA in the subject is about ≥35 ng/mL. In certain embodiments, prior to treatment the circulating CEA in the subject is about ≥40 ng/mL. In certain embodiments, prior to treatment the circulating CEA in the subject is about ≥45 ng/mL. In certain embodiments, prior to treatment the circulating CEA in the subject is about ≥50 ng/mL. In certain embodiments, prior to treatment the circulating CEA in the subject is about ≥55 ng/mL. In certain embodiments, prior to treatment the circulating CEA in the subject is about ≥60 ng/mL. In certain embodiments, prior to treatment the circulating CEA in the subject is about ≥61 ng/mL. In certain embodiments, prior to treatment the circulating CEA in the subject is about ≥62 ng/mL. In certain embodiments, prior to treatment the circulating CEA in the subject is about ≥63 ng/mL. In certain embodiments, prior to treatment the circulating CEA in the subject is about ≥64 ng/mL. In certain embodiments, prior to treatment the circulating CEA in the subject is about ≥65 ng/mL. In certain embodiments, prior to treatment the circulating CEA in the subject is about ≥66 ng/mL. In certain embodiments, prior to treatment the circulating CEA in the subject is about ≥67 ng/mL. In certain embodiments, prior to treatment the circulating CEA in the subject is about ≥68 ng/mL. In certain embodiments, prior to treatment the circulating CEA in the subject is about ≥69 ng/mL. In certain embodiments, prior to treatment the circulating CEA in the subject is about ≥70 ng/mL. In certain embodiments, prior to treatment the circulating CEA in the subject is about ≥71 ng/mL. In certain embodiments, prior to treatment the circulating CEA in the subject is about ≥72 ng/mL. In certain embodiments, prior to treatment the circulating CEA in the subject is about ≥73 ng/mL. In certain embodiments, prior to treatment the circulating CEA in the subject is about ≥74 ng/mL. In certain embodiments, prior to treatment the circulating CEA in the subject is about ≥75 ng/mL. In certain embodiments, prior to treatment the circulating CEA in the subject is about ≥76 ng/mL. In certain embodiments, prior to treatment the circulating CEA in the subject is about ≥77 ng/mL. In certain embodiments, prior to treatment the circulating CEA in the subject is about ≥78 ng/mL. In certain embodiments, prior to treatment the circulating CEA in the subject is about ≥79 ng/mL. In certain embodiments, prior to treatment the circulating CEA in the subject is about ≥80 ng/mL. In certain embodiments, prior to treatment the circulating CEA in the subject is about ≥85 ng/mL. In certain embodiments, prior to treatment the circulating CEA in the subject is about ≥90 ng/mL. In certain embodiments, prior to treatment the circulating CEA in the subject is about ≥95 ng/mL. In certain embodiments, prior to treatment the circulating CEA is about ≥100 ng/mL.

In an embodiment, the anti-CEACAM5 antibody is tusamitamab.

In certain embodiments, the ADC is tusamitamab ravtansine.

In certain embodiments, the tusamitamab ravtansine is administered in a dose of about 60 mg/m² to about ≥190 mg/m². In certain embodiments, the tusamitamab ravtansine is administered in a dose of 60 mg/m² to 190 mg/m².

In certain embodiments, the tusamitamab ravtansine is administered in a dose of about 60 mg/m². In certain embodiments, the tusamitamab ravtansine is administered in a dose of about ≥70 mg/m². In certain embodiments, the tusamitamab ravtansine is administered in a dose of about ≥80 mg/m². In certain embodiments, the tusamitamab ravtansine is administered in a dose of about ≥90 mg/m². In certain embodiments, the tusamitamab ravtansine is administered in a dose of about ≥100 mg/m². In certain embodiments, the tusamitamab ravtansine is administered in a dose of about ≥110 mg/m². In certain embodiments, the tusamitamab ravtansine is administered in a dose of about ≥120 mg/m². In certain embodiments, the tusamitamab ravtansine is administered in a dose of about ≥130 mg/m². In certain embodiments, the tusamitamab ravtansine is administered in a dose of about ≥140 mg/m². In certain embodiments, the tusamitamab ravtansine is administered in a dose of about ≥150 mg/m². In certain embodiments, the tusamitamab ravtansine is administered in a dose of about ≥160 mg/m². In certain embodiments, the tusamitamab ravtansine is administered in a dose of about ≥170 mg/m². In certain embodiments, the tusamitamab ravtansine is administered in a dose of about 180 mg/m². In certain embodiments, the tusamitamab ravtansine is administered in a dose of about ≥190 mg/m². In certain embodiments, the tusamitamab ravtansine is administered in a dose of about ≥200 mg/m².

In certain embodiments, the tusamitamab ravtansine is administered in a dose of 90 mg/m². In certain embodiments, the tusamitamab ravtansine is administered in a dose of 100 mg/m². In certain embodiments, the tusamitamab ravtansine is administered in a dose of 110 mg/m². In certain embodiments, the tusamitamab ravtansine is administered in a dose of 120 mg/m². In certain embodiments, the tusamitamab ravtansine is administered in a dose of 130 mg/m². In certain embodiments, the tusamitamab ravtansine is administered in a dose of 140 mg/m². In certain embodiments, the tusamitamab ravtansine is administered in a dose of 150 mg/m². In certain embodiments, the tusamitamab ravtansine is administered in a dose of 160 mg/m². In certain embodiments, the tusamitamab ravtansine is administered in a dose of 170 mg/m². In certain embodiments, the tusamitamab ravtansine is administered in a dose of 180 mg/m². In certain embodiments, the tusamitamab ravtansine is administered in a dose of 190 mg/m². In certain embodiments, the tusamitamab ravtansine is administered in a dose of 200 mg/m².

In certain embodiments, the tusamitamab ravtansine is administered about once every two weeks. In certain embodiments, the tusamitamab ravtansine is administered about once every three weeks. In certain embodiments, the tusamitamab ravtansine is administered about once every four weeks. In certain embodiments, the tusamitamab ravtansine is administered about once every five weeks. In certain embodiments, the tusamitamab ravtansine is administered about once every six weeks.

In certain embodiments, the tusamitamab ravtansine is administered once every two weeks. In certain embodiments, the tusamitamab ravtansine is administered once every three weeks. In certain embodiments, the tusamitamab ravtansine is administered once every four weeks. In certain embodiments, the tusamitamab ravtansine is administered once every five weeks. In certain embodiments, the tusamitamab ravtansine is administered once every six weeks.

In certain embodiments, the tusamitamab ravtansine is administered in a dose of about 100-200 mg/m² about once every two weeks. In certain embodiments, the tusamitamab ravtansine is administered in a dose of about 100-200 mg/m² once every two weeks.

In certain embodiments, the tusamitamab ravtansine is administered in a dose of 100-200 mg/m² about once every two weeks. In certain embodiments, the tusamitamab ravtansine is administered in a dose of 100-200 mg/m² once every two weeks.

In certain embodiments, the tusamitamab ravtansine is administered in a dose of about 100 mg/m² about once every two weeks. In certain embodiments, the tusamitamab ravtansine is administered in a dose of about 100 mg/m² once every two weeks.

In certain embodiments, the tusamitamab ravtansine is administered in a dose of 100 mg/m² about once every two weeks. In certain embodiments, the tusamitamab ravtansine is administered in a dose of 100 mg/m² once every two weeks.

In certain embodiments, the tusamitamab ravtansine is administered in a dose of about 100-200 mg/m² about once every three weeks. In certain embodiments, the tusamitamab ravtansine is administered in a dose of about ≥100-200 mg/m² once every three weeks.

In certain embodiments, the tusamitamab ravtansine is administered in a dose of 100-200 mg/m² about once every three weeks. In certain embodiments, the tusamitamab ravtansine is administered in a dose of 100-200 mg/m² once every three weeks.

In certain embodiments, the tusamitamab ravtansine is administered in a dose of about 100 mg/m² about once every three weeks. In certain embodiments, the tusamitamab ravtansine is administered in a dose of about ≥100 mg/m² once every three weeks.

In certain embodiments, the tusamitamab ravtansine is administered in a dose of 100 mg/m² about once every three weeks. In certain embodiments, the tusamitamab ravtansine is administered in a dose of 100 mg/m² once every three weeks.

In certain embodiments, the tusamitamab ravtansine is administered in a dose of about 100-200 mg/m² about once every six weeks. In certain embodiments, the tusamitamab ravtansine is administered in a dose of about ≥100-200 mg/m² once every six weeks.

In certain embodiments, the tusamitamab ravtansine is administered in a dose of 100-200 mg/m² about once every six weeks. In certain embodiments, the tusamitamab ravtansine is administered in a dose of 100-200 mg/m² once every six weeks.

In certain embodiments, the tusamitamab ravtansine is administered in a dose of about 100 mg/m² about once every six weeks. In certain embodiments, the tusamitamab ravtansine is administered in a dose of about ≥100 mg/m² once every six weeks.

In certain embodiments, the tusamitamab ravtansine is administered in a dose of 100 mg/m² about once every six weeks. In certain embodiments, the tusamitamab ravtansine is administered in a dose of 100 mg/m² once every six weeks.

In certain embodiments, the method further comprises administering to the subject an effective amount of at least one additional agent effective to treat the cancer.

In certain embodiments, the additional agent is selected from the group consisting of an immune checkpoint inhibitor (ICI), a platinum-based chemotherapy (e.g., cisplatin or carboplatin), pemetrexed, anti-VEGFR2, FOLFOX, FOLFIRI, TAS-102, anti-EGFR, and any combination thereof.

In certain embodiments, the ICI is an anti-PD-1 antibody.

In certain embodiments, the anti-PD-1 antibody is selected from the group consisting of pembrolizumab, nivolumab, cemiplimab, sintilimab, dostarlimab, and tislelizumab.

In certain embodiments, the anti-PD-1 antibody is pembrolizumab.

In certain embodiments, the ICI is an anti-PD-L1 antibody.

In certain embodiments, the anti-PD-L1 antibody is selected from the group consisting of atezolizumab, avelumab, durvalumab, envafolimab, BMS-936559, CK-301, CS-1001, SHR-1316 (HTI-1088), CBT-502 (TQB-2450), and any combination thereof.

In certain embodiments, the anti-PD-L1 antibody is selected from the group consisting of atezolizumab, avelumab, and durvalumab.

In certain embodiments, the method comprises administering to the subject an effective amount of tusamitamab ravtansine and pembrolizumab.

In certain embodiments, the method further comprises administering to the subject an effective amount of a platinum-based chemotherapy.

In certain embodiments, the platinum-based chemotherapy is selected from cisplatin and carboplatin.

In certain embodiments, the method further comprises administering to the subject an effective amount of pemetrexed.

In certain embodiments, the method comprises administering to the subject an effective amount of tusamitamab ravtansine, pembrolizumab, and cisplatin.

In certain embodiments, the method comprises administering to the subject an effective amount of tusamitamab ravtansine, pembrolizumab, cisplatin, and pemetrexed.

In certain embodiments, the method comprises administering to the subject an effective amount of tusamitamab ravtansine, pembrolizumab, and carboplatin.

In certain embodiments, the method comprises administering to the subject an effective amount of tusamitamab ravtansine, pembrolizumab, carboplatin, and pemetrexed.

In certain embodiments, the additional agent is an anti-VEGFR2. In an embodiment, the anti-VEGFR2 is ramucirumab (CYRAMZA®).

In certain embodiments, the additional agent is FOLFOX. FOLFOX is a chemotherapy regimen comprising folinic acid (leucovorin), fluorouracil (5-FU), and oxaliplatin.

In certain embodiments, the additional agent is FOLFIRI. FOLFIRI is a chemotherapy regimen comprising folinic acid (leucovorin), fluorouracil (5-FU), and irinotecan (ONIVYDE®).

In certain embodiments, the additional agent is TAS-102. TAS-102 is a combination of trifluridine and tipiracil hydrochloride.

In certain embodiments, the additional agent is an anti-EGFR. In certain embodiments, the anti-EGFR is selected from cetuximab (ERBITUX®) and necitumumab (PORTRAZZA®).

In certain embodiments, the subject has moderate CEACAM5 expression on tumor cells (≥2+ intensity in ≥1% and <50% of cells) as measured by immunohistochemistry, and circulating CEA about ≥100 ng/mL, and the ADC is tusamitamab ravtansine.

In certain embodiments, the subject has advanced or metastatic cancer, moderate CEACAM5 expression on tumor cells (≥2+ intensity in ≥1% and <50% of cells) as measured by immunohistochemistry, and circulating CEA about ≥100 ng/mL, and the ADC is tusamitamab ravtansine.

In certain embodiments, the subject has negative or low CEACAM5 expression on tumor cells (≥2+ intensity in <1% of cells) as measured by immunohistochemistry, and circulating CEA about ≥100 ng/mL, and the ADC is tusamitamab ravtansine.

In certain embodiments, the subject has advanced or metastatic cancer, negative or low CEACAM5 expression on tumor cells (≥2+ intensity in <1% of cells) as measured by immunohistochemistry, and circulating CEA about ≥100 ng/mL, and the ADC is tusamitamab ravtansine.

In certain embodiments, the subject has NSQ NSCLC, moderate CEACAM5 expression on tumor cells (≥2+ intensity in ≥1% and <50% of cells) as measured by immunohistochemistry, and circulating CEA about ≥100 ng/mL, and the ADC is tusamitamab ravtansine.

In certain embodiments, the subject has advanced or metastatic NSQ NSCLC, moderate CEACAM5 expression on tumor cells (≥2+ intensity in ≥1% and <50% of cells) as measured by immunohistochemistry, and circulating CEA about ≥100 ng/mL, and the ADC is tusamitamab ravtansine.

In certain embodiments, the subject has NSQ NSCLC, negative or low CEACAM5 expression on tumor cells (≥2+ intensity in <1% of cells) as measured by immunohistochemistry, and circulating CEA about ≥100 ng/mL, and the ADC is tusamitamab ravtansine.

In certain embodiments, the subject has advanced or metastatic NSQ NSCLC, negative or low CEACAM5 expression on tumor cells (≥2+ intensity in <1% of cells) as measured by immunohistochemistry, and circulating CEA about ≥100 ng/mL, and the ADC is tusamitamab ravtansine.

### VARIOUS EMBODIMENTS OF THE DISCLOSURE

In embodiment one, the disclosure relates to a circulating carcinoembryonic antigen (CEA) for use as a biomarker in a method of treating a cancer in a subject, the method comprising

measuring circulating carcinoembryonic antigen (CEA) in a subject in need of treatment of cancer; and

administering to the subject an effective amount of an antibody-drug conjugate (ADC) comprising an anti-CEACAM5 antibody if the circulating CEA in the subject is about ≥5 ng/mL,

thereby treating the cancer.

In embodiment 2, the disclosure relates to a circulating carcinoembryonic antigen (CEA) for use as a biomarker in a method of treating a cancer in a subject, the method comprising

selecting a subject having cancer with a circulating carcinoembryonic antigen (CEA) about ≥5 ng/mL; and

administering to the subject an effective amount of an antibody-drug conjugate (ADC) comprising an anti-CEACAM5 antibody,

thereby treating the cancer.

In embodiment 3, the disclosure relates to a circulating carcinoembryonic antigen (CEA) for use as a biomarker in a method of treating a cancer in a subject, the method comprising

measuring a first circulating carcinoembryonic antigen (CEA) in a subject in need of treatment of cancer;

administering to the subject a first effective amount of an antibody-drug conjugate (ADC) comprising an anti-CEACAM5 antibody if the first measured circulating CEA in the subject prior to the administering is about ≥5 ng/mL;

measuring a second circulating CEA in the subject after the administering step; and

administering to the subject a second effective amount of the ADC if the second measured circulating CEA in the subject is less than the first measured circulating CEA,

thereby treating the cancer.

In embodiment 4, the disclosure relates to a circulating carcinoembryonic antigen (CEA) of any one of embodiments 1-3, wherein the anti-CEACAM5 antibody comprises a HCDR1 having the amino acid sequence of SEQ ID NO: 1, a HCDR2 having the amino acid sequence of SEQ ID NO: 2, a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5.

In embodiment 5, the disclosure relates to a circulating carcinoembryonic antigen (CEA) of any one of embodiments 1-4, wherein the cytotoxic agent being maytansinoid or maytansinoid analog.

In embodiment 6, the disclosure relates to an antibody-drug conjugate (ADC) comprising an anti-CEACAM5 antibody conjugated to a cytotoxic agent, for use in a method of treating a cancer in a subject in need thereof,

the anti-CEACAM5 antibody comprising a HCDR1 having the amino acid sequence of SEQ ID NO: 1, a HCDR2 having the amino acid sequence of SEQ ID NO: 2, a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5,

the cytotoxic agent being maytansinoid or maytansinoid analog, and

the method comprising measuring circulating carcinoembryonic antigen (CEA) in said subject and administering to said subject an effective amount of said ADC if the circulating CEA in said subject is about ≥5 ng/mL, thereby treating the cancer.

In embodiment 7, the disclosure relates to a use of an antibody-drug conjugate (ADC) comprising an anti-CEACAM5 antibody conjugated to a cytotoxic agent, for manufacturing a medicament for use in a method of treating a cancer in a subject in need thereof,

the anti-CEACAM5 antibody comprising a HCDR1 having the amino acid sequence of SEQ ID NO: 1, a HCDR2 having the amino acid sequence of SEQ ID NO: 2, a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5,

the cytotoxic agent being maytansinoid or maytansinoid analog, and

the method comprising measuring circulating carcinoembryonic antigen (CEA) in said subject and administering to said subject an effective amount of said medicament if the circulating CEA in said subject is about ≥5 ng/mL, thereby treating the cancer.

In embodiment 8, the disclosure relates to a method of treating a cancer in a subject in need thereof, the method comprising the steps of:

measuring circulating carcinoembryonic antigen (CEA) in said subject, and

administering to said subject an effective amount of an antibody-drug conjugate ADC if the circulating CEA in said subject is about ≥5 ng/mL, thereby treating the cancer,

wherein the antibody-drug conjugate (ADC) comprises an anti-CEACAM5 antibody conjugated to a cytotoxic agent,

wherein the anti-CEACAM5 antibody comprises a HCDR1 having the amino acid sequence of SEQ ID NO: 1, a HCDR2 having the amino acid sequence of SEQ ID NO: 2, a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5, and

wherein the cytotoxic agent is maytansinoid or a maytansinoid analog.

In embodiment 9, the disclosure relates to any one of embodiments 4-8 wherein the method comprises:

selecting a subject having cancer with a circulating carcinoembryonic antigen (CEA) about ≥5 ng/mL; and

administering to the subject an effective amount of an antibody-drug conjugate (ADC) comprising an anti-CEACAM5 antibody,

thereby treating the cancer.

In embodiment 10, the disclosure relates to the disclosure relates to any one of embodiments 4-8 wherein the method comprises:

measuring a first circulating carcinoembryonic antigen (CEA) in a subject in need of treatment of cancer;

administering to the subject a first effective amount of an antibody-drug conjugate (ADC) comprising an anti-CEACAM5 antibody if the first measured circulating CEA in the subject prior to the administering is about ≥5 ng/mL;

measuring a second circulating CEA in the subject after the administering step; and

administering to the subject a second effective amount of the ADC if the second measured circulating CEA in the subject is less than the first measured circulating CEA,

thereby treating the cancer.

In embodiment 11, the disclosure relates to any one of embodiments 1-10, wherein the cancer is selected from the group consisting of colorectal cancer, gastric cancer, gastroesophageal junction cancer, esophageal cancer, lung cancer, uterine cervix cancer, pancreatic cancer, ovarian cancer, thyroid cancer, bladder cancer, endometrial cancer, breast cancer, liver cancer, biliary tract cancer (e.g., cholangiocarcinoma), prostate cancer, and skin cancer.

In embodiment 12, the disclosure relates to any one of embodiments 1-11, wherein the cancer is gastric cancer.

In embodiment 13, the disclosure relates to any one of embodiments 1-12, wherein the cancer is lung cancer.

In embodiment 14, the disclosure relates to any one of embodiments 1-13, wherein the circulating CEA is measured after performing immunohistochemistry analysis of CEACAM5 expression on tumor cells of the subject.

In embodiment 15, the disclosure relates to any one of embodiments 1-14, wherein the subject has negative or low CEACAM5 expression on tumor cells (≥2+ intensity in <1% of cells) as measured by immunohistochemistry, or
the subject has moderate CEACAM5 expression on tumor cells (≥2+ intensity in ≥1% and <50% of cells) as measured by immunohistochemistry, or
the subject has high CEACAM5 expression on tumor cells (≥2+ intensity in ≥50% of cells) as measured by immunohistochemistry.

In embodiment 16, the disclosure relates to any one of embodiments 1-15, wherein prior to treatment the circulating CEA is about ≥20 ng/mL, or
prior to treatment the circulating CEA is about ≥50 ng/mL, or
prior to treatment the circulating CEA is about ≥80 ng/mL, or
prior to treatment the circulating CEA is about ≥100 ng/mL.

In embodiment 17, the disclosure relates to any one of embodiments 1-16, wherein the anti-CEACAM5 antibody is tusamitamab.

In embodiment 18, the disclosure relates to any one of embodiments 1-17, wherein the ADC is tusamitamab ravtansine.

In embodiment 19, the disclosure relates to embodiment 18, wherein the tusamitamab ravtansine is administered in a dose of about ≥100 mg/m² about once every two weeks, or the tusamitamab ravtansine is administered in a dose of about ≥100 mg/m² about once every three weeks.

In embodiment 20, the disclosure relates to any one of embodiments 1-19, further comprising administering to the subject an effective amount of at least one additional agent effective to treat the cancer.

In embodiment 21, the disclosure relates to embodiment 20, wherein the additional agent is selected from the group consisting of an immune checkpoint inhibitor (ICI), a platinum-based chemotherapy, pemetrexed, anti-VEGFR2, FOLFOX, FOLFIRI, TAS-102, anti-EGFR, and any combination thereof.

In embodiment 22, the disclosure relates to embodiment 21, wherein the ICI is an anti-PD-1 antibody or an anti-PD-L1 antibody.

In embodiment 23, the disclosure relates to embodiment 22, wherein the anti-PD-1 antibody is selected from the group consisting of pembrolizumab, nivolumab, cemiplimab, sintilimab, dostarlimab, and tislelizumab.

In embodiment 24, the disclosure relates to embodiment 22, wherein the anti-PD-L1 antibody is selected from the group consisting of atezolizumab, avelumab, and durvalumab.

In embodiment 25, the disclosure relates to any one of embodiments 1-23, comprising administering to the subject an effective amount of tusamitamab ravtansine and pembrolizumab.

In embodiment 26, the disclosure relates to embodiment 25, further comprising administering to the subject an effective amount of a platinum-based chemotherapy.

In embodiment 27, the disclosure relates to embodiment 26, wherein the platinum-based chemotherapy is selected from cisplatin and carboplatin.

In embodiment 28, the disclosure relates to embodiment 27, further comprising administering to the subject an effective amount of pemetrexed.

### EXAMPLES

### Example 1. Background and Study Rationale

In the first-in-human (FIH) TED13751 study, a cohort of 64 participants with heavily pre-treated NSQ NSCLC with high CEACAM5 expression using the IHC method applied at prescreening on archived tumor sample (defined as ≥2+ in intensity in at least 50% of the tumor cells) were treated with tusamitamab ravtansine at 100 mg/m² Q2W. Tusamitamab ravtansine has shown an encouraging anti-tumor activity. This anti-tumor activity was associated with an overall response rate of 20.3% (13 participants who had PR) per Response Evaluation Criteria in Solid Tumours (RECIST) 1.1 (95% CI: 12.27% to 31.71%), warranting further development of tusamitamab ravtansine to treat this patient population. In this cohort of NSQ NSCLC participants with high CEACAM5 expression on tumor (≥2+ in intensity in ≥50%) expansion cohort NSQ NSCLC, 10 out of 13 responding participants had high baseline circulating CEA (≥100 ng/mL). Potential responses to tusamitamab ravtansine have also been evaluated in a cohort of NSQ NSCLC participants with moderate CEACAM5 expression on tumor (≥2+ in intensity in ≥1% and <50% of tumor cells) but only a limited number of participants (7 out of 28 participants) presented high circulating CEA levels at baseline.

The CEACAM5 expression status may have differed between the initial tumor at diagnosis and the tumor at the time of the enrollment in the clinical trial. Even though a correlation is expected between circulating CEA and CEACAM5 expression status on tumor, there are no available data on concomitant CEACAM5 expression on fresh tumor biopsy and circulating CEA. Biomarkers in oncology are most accurate and best correlated with clinical outcomes when they are assessed immediately prior to therapy.

Participants are prescreened in EFC15858 study in second or third-line therapy based on the expression of CEACAM5 that is assessed on archival biopsy at the time of diagnosis, prior to first-line therapy, which can be months or years before enrollment. This is potentially important for participants who are prescreened in EFC15858 and whose diagnostic biopsies are negative, but who could have high CEACAM5 expression at the time of a fresh biopsy, either due to heterogeneous expression of CEACAM5 or upregulation of CEACAM5 expression after first-line therapy.

In the instant study, a fresh biopsy will be proposed (optional) at screening to verify the correlation of CEACAM5 expression at diagnosis and at the time of enrollment as well as the correlation of high circulating CEA and high CEACAM5 expression at the time of enrollment. The fresh biopsy will be assessed retrospectively as due to the heterogenicity of tumors, participants with negative CEACAM5 expression may have high expression on other tumor locations.

The aim of this study is to evaluate if participants with high circulating CEA level at baseline could benefit from tusamitamab ravtansine treatment despite CEACAM5 negative-moderate expression on archival biopsy, assuming that a high circulating CEA level reflects a high CEACAM5 expression on tumor at the time of the enrollment. To this end, the anti-tumor activity is assessed in participants with NSQ NSCLC with high circulating CEA levels despite negative-moderate CEACAM5 expression on tumor by IHC testing in third- or fourth-line treatment.

### Example 2. Assessing CEACAM5 Expression on Tumor Using CEACAM5 IHC Test and Circulating CEA and CEACAM5 Assays

Expression of CEACAM5 on tumor was assessed using CEACAM5 IHC test and circulating CEA and CEACAM5 assays for patients enrolled in clinical trial TED13751 (NCT02187848).

### Methods

### A. Tumor CEACAM5 Immunohistochemistry (IHC) Testing:

CEACAM5 tumor expression was assessed at prescreening on the most recent available archival tumor sample (i.e., archive tumor tissue at diagnosis, archive tumor tissue at surgery, or tumor sample before inclusion in the study and not under anticancer treatment). The level and pattern of CEACAM5 expression in tumor tissues were determined using IHC either run locally at the clinical site (prospective testing) and/or centrally in a laboratory (retrospective or prospective testing). For local assessment, assays were done using anti-CEACAM5 clone 769 antibody provided by Sanofi. Anti-CEACAM5 clone 769 is a murine monoclonal antibody with the same specificity as tusamitamab ravtansine to the CEACAM5 target. For central assessment, samples were first analyzed with a validated assay using Sanofi anti-CEACAM5 clone 769 antibody run on Techmate platform. Then, in a second analysis a validated CEACAM5 IHC assay was run on Dako/Agilent Autostainer Link 48 IHC platform using the same anti-CEACAM5 antibody at the same concentration. For both assays, interpretation of CEACAM5 reactivity was performed by a board-certified pathologist using semi-quantitative Percent Scores (calculated by summing the percentages of intensities ≥2+) or H-score for CEACAM5 plasma membrane staining (whole or polarized) in tumor cells. Cytoplasmic staining was also evaluated.

### B. Circulating CEA Assay:

Circulating CEA was determined locally using the assay which was of routine use in the institution. In vitro diagnostic CEA assays measure CEA not claiming specificity for CEACAM5. Samples for circulating CEA were collected at baseline (at any time during screening and before first administration of tusamitamab ravtansine), and then, following the same schedule as tumor assessment during treatment, and at disease progression.

### C. Circulating CEACAM5 Assay:

Circulating CEACAM5 was determined centrally at prescreening, baseline (either within 7 days of administration or at Cycle 1, Day 1 (CID1) and at the same timepoints of PK sampling during cycle 1 and before each administration of tusamitamab ravtansine. Central testing specific to circulating CEACAM5 levels was done at a sponsor laboratory. The assay was a quantitative sandwich enzyme immunoassay ELISA method using a rat monoclonal anti-human CEACAM5 antibody (clone GFR44) for capture and a sheep polyclonal anti-human CEACAM5 antibody conjugated to biotin for the detection. In vitro evaluation of interference with tusamitamab ravtansine at concentration ranging from 0.100 ng/mL to 500 μg/mL showed no impact on the quantitation of CEACAM5 in human plasma. The quantitation range was from 120 μg/mL to 750,000,000 μg/mL (750 μg/mL).

### Results

### A. Circulating CEA/Circulating CEACAM5 Correlation:

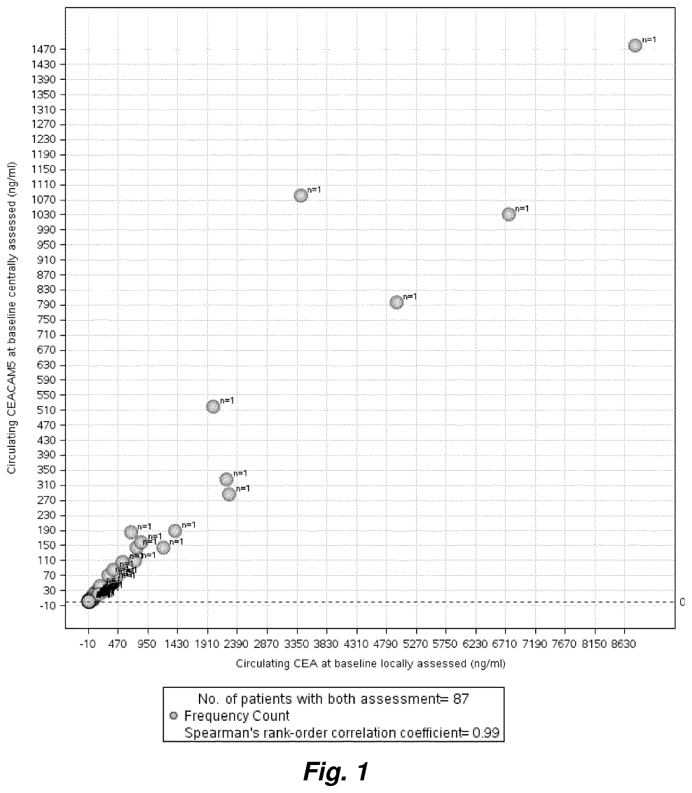

Circulating CEA and circulating CEACAM5 levels at baseline for high and moderate NSQ NSCLC cohorts are shown in FIG. 1. The correlation between baseline circulating CEA and circulating CEACAM5 level was very strong (Spearman's rank order correlation coefficient of 0.99) indicating that the entity measured as circulating CEA in this patient population is closely related to circulating CEACAM5.

### B. Baseline Circulating CEA Levels and Responses:

Overall response rate (ORR) by circulating CEA levels is presented in Table 1. Forty percent of the high expressers presented a high circulating CEA level at baseline (≥100 μg/L). Ten out of the 13 responders (77%) had baseline circulating CEA level ≥100 μg/L, indicating that high circulating CEA levels may be predictive of response.

TABLE 1
Summary of Overall Response Rate with 95% CI
by circulating CEA levels (all treated population -
NSQ NSCLC High expressers cohort)

| | Tusamitamab ravtansine | | |
|---|---|---|---|
| | 100 mg/m² Q2W | | |
| | (N = 64) | | |
| | | Responses | |
| | Number | (n[%]) | 95% CI^{a} |
| Circulating CEA locally assessed | | | |
| at baseline | | | |
| Number | 62 | | |
| ≤3 μg/L | 4 | 0 | |
| >3 μg/L | 58 | 13 (22.4%) | (13.59% to |
| | | | 34.66%) |
| Circulating CEA locally assessed | | | |
| at baseline | | | |
| Number | 62 | | |
| <5 μg/L | 9 | 2 (22.2%) | (6.32% to |
| | | | 54.74%) |
| ≥5 μg/L | 53 | 11 (20.8%) | (12.00% to |
| | | | 33.46%) |
| Circulating CEA locally assessed | | | |
| at baseline | | | |
| Number | 62 | | |
| <100 μg/L | 37 | 3 (8.1%) | (2.80% to |
| | | | 21.30%) |
| ≥100 μg/L | 25 | 10 (40.0%) | (23.40% to |
| | | | 59.26%) |

^{a}Estimated by Wilson score interval

### C. CEACAM5 Tumor Expression/Circulating CEA Correlation:

Correlation between circulating CEA level at baseline and CEACAM5 expression by immunohistochemistry for high and moderate NSQ NSCLC cohorts is presented in Table 2.

TABLE 2
Correlation between circulating CEA at baseline and
percent of positive cells of CEACAM5 at intensity ≥2+,
overall membrane (biomarker evaluable population -
high and moderate NSQ NSCLC cohorts)

| | tusamitamab |
|---|---|
| | ravtansine |
| | 100 mg/m² |
| | Q2W (N = 64) |
| High expresser cohort | |
| Circulating CEA at baseline and percent of positive cells | |
| at intensity ≥2+, archival biopsy, overall membrane | |
| Number | 62 |
| Spearman's rank-order correlation coefficient | 0.402 |
| Moderate expresser cohort | |
| Circulating CEA at baseline and percent of positive cells | |
| at intensity ≥2+, archival biopsy, overall membrane | |
| Number | 28 |
| Spearman's rank-order correlation coefficient | 0.343 |

Correlation between circulating CEA and CEACAM5 expression in tumor tissue was weak for the two cohorts (Spearman's rank-order correlation coefficient of 0.402 for high expresser cohort and 0.343 for moderate expresser cohort). This difference may be due to the different timepoint of collection between the three samples (archive samples for IHC that can have been sampled well before study start) and because CEACAM5 expression may be heterogeneous in the tumor or may differ between primary tumor and metastases. Circulating CEA as a systemic evaluation may reflect total CEACAM5 originating from several lesions (e.g., primary or metastases).

### Example 3. Study Design

This is an open-label, non-randomized, dose escalation, safety, pharmacokinetic and antitumor activity evaluation of tusamitamab ravtansine administered as a single agent by IV infusion, q2w or q3w (1 cycle=3 weeks), in adults with advanced solid tumors. Escalation phase has 3 different cohorts as below: a Main dose escalation cohort with every 2 weekly administration of tusamitamab ravtansine, an Escalation bis cohort with every 2 weekly administration of tusamitamab ravtansine with a loading dose at Cycle 1 only, and a Dose escalation q3w cohort with every 3 weekly administration of tusamitamab ravtansine. The study will be divided in three parts: The Escalation Phase and the Expansion Phase.

During the Escalation Phase, the population to be treated will be enriched, but not restricted, for patients with tumor types known to express CEACAM5; confirmation of the CEACAM5 expression will be done retrospectively using IHC on the most recent archival tissue samples and in a central laboratory. Expression of circulating CEACAM5 may be used for enrichment.

The expansion phase will be conducted with every 2 weekly dosing schedule. During the Expansion Phase, the population to be treated will be restricted to patients with advanced diseases in colorectal cancer (CRC), NSQ NSCLC, small cell lung cancer (SCLC), and gastric adenocarcinoma (including signet-ring cell carcinoma subtype as well as esophago-gastric junction (EGJ) adenocarcinomas of the Siewert Types II and III). CEACAM5 expression of ≥2+ in intensity involving ≥50% of the tumor cell population will be documented during pre-screening on the most recent archival tissue sample and using local IHC evaluation for patients potentially eligible for study treatment in the gastric adenocarcinoma cohort. There are 2 independent NSQ NSCLC expansion phase cohorts: The first one (Lung cohort) includes patients with high CEACAM5 expression (≥2+ in intensity involving at least 50% of the tumor cell population). The second cohort (Lung bis cohort) will include patients that pre-screened positive at moderate intensity (≥2+ between ≥1% to <50% of the tumor cell population). The SCLC cohort includes patients with CEACAM5 expression of ≥2+ in intensity involving ≥1% of the tumor cell population. For investigational centers that do not have the CEACAM5 assay-based on tusamitamab ravtansine monoclonal antibody—implemented on their local IHC platform a pre-screening assessment of tumor CEACAM5 expression will be conducted centrally. Full screening will only be done for archival cases meeting the above definition. No CEACAM5 pre-screening is required for CRC patients potentially eligible for the CRC expansion cohort. The level of CEACAM5 expression will be documented essentially retrospectively and centrally on both archival and fresh (baseline sample) tumor tissues. Confirmation of CEACAM5 tumor expression will be done retrospectively in a central laboratory on fresh tumor tissue collected at baseline (mandatory biopsy in the Expansion Phase only, on all patients with CRC and gastric cancer, and on NSQ NSCLC as well as SCLC patients with a lesion amenable to biopsy). If sufficient archived tumor material is available, a central evaluation will also be done retrospectively to gain knowledge on the variability in evaluation of the expression. The results of the retrospective analyses will have no impact on patients' treatment. It will be used for the better interpretation of the overall response, and as the baseline for comparison with CEACAM5 expression upon progression (exploration for loss of CEACAM5 as mechanism of acquired resistance).

Based on this purpose, circulating CEACAM5 will be collected in all pre-screened NSCLC and SCLC patients to capture data also in pre-screened failed patients (i.e., CEACAM5 negative or expression <2+ intensity).

Approximately 3 mL of blood will be withdrawn for this sample and tested at central laboratory during the pre-screening visit. As this is prospective descriptive diagnostic analyses, there is no statistical power for potential patients to be included and all new pre-screened patients with NSQ NSCLC and SCLC will be candidates for this pre-screening CEACAM5 assessment part. Roughly 562 patients (334 patients with NSQ NSCLC and 228 patients with SCLC) will be pre-screenedbased on potential targeted patients to be enrolled to NSQ NSCLC and SCLC cohorts.

The dose escalation decision will be based on the dose limiting toxicities (DLTs) observed during Cycle 1+Cycle 2 in q2w cycle and during Cycle 1 in q3w cycle, whether related or not to the investigational medicinal product (IMP) in the absence of clear evidence to the contrary after validation by the Study Committee, and if not related to the progression of disease. Before escalating the tusamitamab ravtansine dose to the next level, the safety data, and especially DLTs, ocular and cardiac adverse events (AEs) will be reviewed by the Study Committee. Cumulative toxicities observed on subsequent administrations will also be considered for the dose escalation process and the dose selection decision. Dose escalation or dose decrease decisions will be based on the assessment of DLTs, in agreement between at least the Sponsor and the Principal Investigators.

The safety of a fixed dose of tusamitamab ravtansine administered once q2w (Day 1 of each 14-day cycle) will be assessed in a Main Dose Escalation process using an accelerated dose escalation for the first 3 dose levels (DLs), followed by a Bayesian Escalation with Overdose Control (EWOC).

For the accelerated dose escalation phase, as well as for the Bayesian EWOC, a minimum of three cycles duration (˜4 weeks) will be mandatory between the last patient who has received Day 1 of Cycle 1 at DL n, and the first patient who will receive Day 1 of Cycle 1 at DL n+1. The dose determined to be the maximum tolerated dose (MTD) will be tested further in the Expansion cohorts.

The safety of a loading dose of tusamitamab ravtansine on Day 1 of Cycle 1 followed by administration of 100 mg/m² (determined as being the MTD in the Main Escalation) at all subsequent cycles will be assessed in a Dose Escalation bis, which will be using a Bayesian EWOC process, and will be carried out in parallel to the Expansion Phase. The first loading dose tested will be 120 mg/m²; if this first dose is safe, then dose escalation will proceed to test next 135 mg/m², and 150 mg/m². If no DLT occur at 150 mg/m², Study Committee may decide to evaluate higher DLs of 170, 190, and 210 mg/m², if the Bayesian rules allow it. If no DLT occur at 210 mg/m², the safety of this loading dose (MTD or safe maximum administered dose (MAD)) will be taken into consideration for the recommendation of the Phase 2 dose at the end of the study. The determined MTD or safe MAD will be used for CRC-L cohort.

The safety of tusamitamab ravtansine administered once q3w (Day 1 of each 21-day cycle) will be assessed in a Dose Escalation q3w process using the Bayesian EWOC design used as the Main Dose Escalation. The DLs tested in the model will be 120, 150, 170, 190, and 210 mg/m². The DLs 190 and 210 mg/m² are optional DLs and escalation to these DLs will be determined by Study Committee based on DLT during Cycle 1 as well as cumulative safety data and PK data and Bayesian model.

### Inclusion Criteria

Locally advanced or metastatic solid malignant tumor disease for which, in the judgement of the Investigator, no standard alternative therapy is available and meeting the following inclusion criteria.

At least 6×5 μm slides plus an additional number of slides that can be either 3×10 μm (best), or 6×5 μm, or equivalent to keep the same total amount of material needed, from formalin-fixed paraffin-embedded (FFPE) archival tissue should be available for local testing at the site and/or shipment to the Sponsor, or laboratory designated by the Sponsor, evaluation of tumor CEACAM5 expression (retrospectively in the Escalation Phase and prospectively in the Expansion Phase) and exploration of other predictive biomarkers of response. If less material is available, patient could still be eligible after discussion with the Sponsor who will assess and confirm that there is sufficient relevant material for key evaluations.

For participants to the Escalation Phase cohorts (Main, bis and q3w): Inclusion is enriched for (although not restricted to) tumors expressing or likely to be expressing CEACAM5 which include:
Malignant diseases with high prevalence of CEACAM5 expression, i.e., CRC, NSQ NSCLC of the adenocarcinoma or large cell subtype, gastric adenocarcinoma including signet cell carcinoma and EGJ adenocarcinomas of the Siewert Types II and III, or
Malignant diseases with various degree of prevalence and intensity of CEACAM5 expression including: cervix squamous cell carcinoma, pancreas adenocarcinoma, bladder transitional cell carcinoma, cholangiocarcinoma, endometrial adenocarcinoma, and epithelial ovarian cancer, or
Circulating CEA levels >5 ng/mL, as demonstrated by local testing.

For participants to the Expansion Phase cohorts: Inclusion is restricted to:
Patients with either NSCLC of the non-squamous sub-types or gastric adenocarcinoma including the signet-ring cell carcinoma subtype as well as esophagogastric junction (EGJ; equivalently, gastroesophageal junction (GEJ)) adenocarcinomas of the Siewert Types II and III. For the gastric adenocarcinoma cohort, CEACAM5 expression in the most recent FFPE archival tumor tissue sample is ≥2+ in intensity in at least 50% of the tumor cell population, as demonstrated prospectively by local IHC evaluation. There are three independent cohorts in NSQ NSCLC expansion phase, based on local or central CEACAM5 expression assessment on archival tumor tissue: The first one (Lung) includes patients with CEACAM5 expression at ≥2+ in intensity involving at least 50% of the tumor cell population. The second independent cohort (Lung bis) includes patients that pre-screened positive at the intensity of ≥2+ in ≥1% to <50% of the tumor cell population.
Patients with SCLC with CEACAM5 expression in the most recent FFPE archival tumor tissue sample, is ≥2+ in intensity involving ≥1% of the tumor cell population, as demonstrated prospectively by local or central IHC evaluation.
Or to all corners CRC patients (including patients who pre-screened CEACAM5 positive, regardless of the level of expression). Once the Expansion Phase has started, then CRC patients must be included in the Expansion CRC cohort in priority, and not in the Dose Escalation bis that will recruit in parallel, unless non-eligible (no target lesion or no lesion amenable to biopsy).
At least one measurable lesion by Response Evaluation Criteria in Solid Tumors version 1.1 (RECIST v1.1) in the Expansion Phase only.
At least one lesion amenable to biopsy (Expansion cohort only—CRC and gastric cancer only). Patient must consent to a baseline biopsy for retrospective confirmation of tumor CEACAM5 expression before treatment initiation, except if NSCLC or SCLC without lesion amenable to biopsy).

### Exclusion Criteria

### Life Expectancy <12 Weeks.

Known or symptomatic brain metastasis (other than totally resected or previously irradiated and non-progressive/relapsing) or lepto-meningeal carcinomatosis.

Concurrent treatment with any other anticancer therapy.

Prior maytansinoid treatments (DM1 or DM4 antibody drug conjugates).

### Treatments

Tusamitamab ravtansine is supplied as a 25 mL extractable volume of concentrate for solution for infusion of 125 mg (5 mg/mL) contained in a 30 mL type I glass vial.

The initial starting DL is 5 mg/m² to be administered q2w. In the Dose Escalation q3w cohort, the starting DL is 120 mg/m². The patient's body surface area (BSA) will be calculated using their height and actual body weight. For patients with a BSA >2.2 m², the dose will be calculated on the basis of 2.2 m² BSA.

Pre-medication with Histamine H1 antagonist (diphenylhydramine 50 mg PO or equivalent [e.g., dexchlorpheniramine] given approximately 1 hour before tusamitamab ravtansine administration) is required for all patients. Using an infusion-controlled pump, tusamitamab ravtansine will be administered by IV infusion at a rate of 2.5 mg/min for the first 30 minutes and then increased to 5 mg/min in the absence of hypersensitivity reactions.

### Efficacy

### Pharmacodynamic Biomarker Evaluation

Tusamitamab ravtansine is a cytotoxic agent with a selective delivery system, and as such, induction of objective responses in the clinic is expected. A single administration of tusamitamab ravtansine induces regressions in relevant experimental model.

However, specificities linked to the CEACAM5 target would potentially allow a more accurate follow-up of treatment impact on tumor growth by the monitoring CEA levels in the blood. Indeed, measurement of circulating CEA levels is of some use for CRC treatment monitoring. Usually, CEA returns to normal within 1-2 months following surgery and if it does not return, it is indicative of disease recurrence. Similarly, modulation of circulating CEA may be a useful PDy marker of response to tusamitamab ravtansine. Internal preclinical studies assessing circulating CEA in mice engrafted orthotopically with a patient-derived CRC xenograft expressing CEACAM5 showed a good correlation between the level of CEA and tumor burden, and treatment with a single active dose of tusamitamab ravtansine prevented a rise in circulating CEA occurring with tumor growth. The usefulness of monitoring circulating CEA will be explored in this clinical study.

Plasma samples of 2 mL or 3 mL will be collected at baseline and under treatment, for all patients participating in an Expansion cohort for the determination of the modulation of CEA levels under tusamitamab ravtansine treatment. In Cycle 1, samples collection will be matched with PK collection after tusamitamab ravtansine starting from the 6 h time point post infusion. Samples for circulating CEA will be collected once before each subsequent administration, at end of treatment and at follow-up if required. The determination of circulating CEACAM5 level for PDy will be done in a central laboratory designated by the Sponsor, and using a commercial ELISA kit. In order to assess intra-patient variability, one plasma sample will be collected within the 7 days of registration (screening period) and then predose on Day 1 of Cycle 1.

### Criteria for Efficacy Assessment

In the Escalation cohort, the objective response information will be obtained if patients have disease which can readily be measured and re-assessed.

In the Expansion cohorts, the assessment of response to tusamitamab ravtansine is a primary objective. All patients treated in the Expansion Phase must have at least one measurable lesion for inclusion. Tumor assessment will be made at least every 4 cycles. Decision to pursue treatment will be based on the response evaluation made by the Investigator, however, measures of lesions will be collected in the electronic case report forms for a determination of response by Sponsor. A partial or complete response must be confirmed on a second examination done at least 4 weeks apart, in order to be documented as a confirmed response to therapy.

The RECIST v1.1 criteria will be followed for assessment of tumor response.

1-19. (canceled)

20. A method of treating cancer in a subject in need thereof comprising:
measuring circulating carcinoembryonic antigen (CEA) in the subject; and
administering to the subject an effective amount of an antibody-drug conjugate (ADC) if the circulating CEA in the subject is ≥5 ng/mL, thereby treating the cancer,
wherein the ADC comprises an anti-CEACAM5 antibody,
wherein the anti-CEACAM5 antibody comprises a HCDR1 having the amino acid sequence of SEQ ID NO: 1, a HCDR2 having the amino acid sequence of SEQ ID NO: 2, a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5.

21. The method according to claim 20, wherein the antibody is conjugated to an inhibitory growth agent.

22. The method according to claim 20, wherein the antibody is conjugated to a cytotoxic agent.

23. The method according to claim 22, wherein the cytotoxic agent is selected from the group consisting of taxoid, vincas, maytansinoid or maytansinoid analog, tomaymycin or pyrrolobenzodiazepine derivative, cryptophycin derivative, leptomycin derivative, auristatin or dolastatin analog, topoisomerase II inhibitors, DNA alkylating agent, anti-tubulin agent, and CC-1065 or CC-1065 analog.

24. The method according to claim 23, wherein the cytotoxic agent maytansinoid analog is N2′-deacetyl-N2′-(3-mercapto-1-oxopropyl)-maytansine (DM1) or N2′-deacetyl-N-2′(4-methyl-4-mercapto-1-oxopentyl)-maytansine (DM4).

25. The method according to claim 20, wherein the cancer is selected from the group consisting of colorectal cancer, gastric cancer, gastroesophageal junction cancer, esophageal cancer, lung cancer, uterine cervix cancer, pancreatic cancer, ovarian cancer, thyroid cancer, bladder cancer, endometrial cancer, breast cancer, liver cancer, biliary tract cancer (e.g., cholangiocarcinoma), prostate cancer, and skin cancer.

26. The method according to claim 20, wherein the cancer is gastric cancer.

27. The method according to claim 20, wherein the cancer is lung cancer.

28. The method according to claim 20, wherein the cancer is colorectal cancer.

29. The method according claim 20, wherein the circulating CEA is measured after performing immunohistochemistry analysis of CEACAM5 expression on tumor cells of the subject.

30. The method according to claim 20, wherein the subject has negative or low CEACAM5 expression on tumor cells (≥2+ intensity in <1% of cells) as measured by immunohistochemistry, or
the subject has moderate CEACAM5 expression on tumor cells (≥2+ intensity in ≥1% and <50% of cells) as measured by immunohistochemistry, or
the subject has high CEACAM5 expression on tumor cells (≥2+ intensity in ≥50% of cells) as measured by immunohistochemistry.

31. The method according to claim 20, wherein prior to treatment the circulating CEA is ≥20 ng/mL, or
prior to treatment the circulating CEA is ≥50 ng/mL, or
prior to treatment the circulating CEA is ≥80 ng/mL, or
prior to treatment the circulating CEA is ≥100 ng/mL.

32. The method according to claim 20, wherein the anti-CEACAM5 antibody is tusamitamab.

33. The method according to claim 20, wherein the ADC is tusamitamab ravtansine.

34. The method according to claim 33, wherein the tusamitamab ravtansine is administered in a dose of ≥100 mg/m² about once every two weeks, or the tusamitamab ravtansine is administered in a dose of ≥100 mg/m² about once every three weeks.

35. The method according to claim 20, further comprising administering to the subject an effective amount of at least one additional agent effective to treat the cancer.

36. The method according to claim 35, wherein the additional agent is selected from the group consisting of an immune checkpoint inhibitor (ICI), a platinum-based chemotherapy, pemetrexed, anti-VEGFR2, FOLFOX, FOLFIRI, TAS-102, anti-EGFR, and any combination thereof.

37. The method according to claim 36, wherein the ICI is an anti-PD-1 antibody or an anti-PD-L1 antibody.

38. The method according to claim 37, wherein the anti-PD-1 antibody is selected from the group consisting of pembrolizumab, nivolumab, cemiplimab, sintilimab, dostarlimab, and tislelizumab.

39. The method according to claim 37, wherein the anti-PD-L1 antibody is selected from the group consisting of atezolizumab, avelumab, and durvalumab.

40. The method according to claim 20, comprising administering to the subject an effective amount of tusamitamab ravtansine and pembrolizumab.

41. The method according to claim 40, further comprising administering to the subject an effective amount of a platinum-based chemotherapy.

42. The method according to claim 41, wherein the platinum-based chemotherapy is selected from cisplatin and carboplatin.

43. The method according to claim 42, further comprising administering to the subject an effective amount of pemetrexed.

44. A method of treating cancer in a subject in need thereof comprising:
measuring circulating carcinoembryonic antigen (CEA) in the subject; and
administering to the subject an effective amount of an antibody-drug conjugate (ADC) if the circulating CEA in the subject is ≥5 ng/mL, thereby treating the cancer,
wherein the ADC comprises an anti-CEACAM5 antibody conjugated to a cytotoxic agent,
wherein the anti-CEACAM5 antibody comprising a HCDR1 having the amino acid sequence of SEQ ID NO: 1, a HCDR2 having the amino acid sequence of SEQ ID NO: 2, a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5, and the cytotoxic agent being maytansinoid or maytansinoid analog.

45. A method of treating cancer in a subject in need thereof comprising:
measuring circulating carcinoembryonic antigen (CEA) in the subject; and
administering to the subject an effective amount of an antibody-drug conjugate (ADC) if the circulating CEA in the subject is ≥30 ng/mL, thereby treating the cancer,
wherein the ADC comprises an anti-CEACAM5 antibody conjugated to a cytotoxic agent,
wherein the anti-CEACAM5 antibody comprising a HCDR1 having the amino acid sequence of SEQ ID NO: 1, a HCDR2 having the amino acid sequence of SEQ ID NO: 2, a HCDR3 having the amino acid sequence of SEQ ID NO: 3, a LCDR1 having the amino acid sequence of SEQ ID NO: 4, a LCDR2 having the amino acid sequence NTR, and a LCDR3 having the amino acid sequence of SEQ ID NO: 5.


SEQUENCE LISTING (9 sequences)

SEQ ID NO: 1 - protein, 8 aa, synthetic construct
  REGION 1..8: note = Synthetic polypeptide
GFVFSSYD

SEQ ID NO: 2 - protein, 8 aa, synthetic construct
  REGION 1..8: note = Synthetic polypeptide
ISSGGGIT

SEQ ID NO: 3 - protein, 13 aa, synthetic construct
  REGION 1..13: note = Synthetic polypeptide
AAHYFGSSGPFAY

SEQ ID NO: 4 - protein, 6 aa, synthetic construct
  REGION 1..6: note = Synthetic polypeptide
ENIFSY

SEQ ID NO: 5 - protein, 9 aa, synthetic construct
  REGION 1..9: note = Synthetic polypeptide
QHHYGTPFT

SEQ ID NO: 6 - protein, 121 aa, synthetic construct
  REGION 1..121: note = Synthetic polypeptide
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYVVGQGTLVTVSS

SEQ ID NO: 7 - protein, 106 aa, synthetic construct
  REGION 1..106: note = Synthetic polypeptide
DIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEI

SEQ ID NO: 8 - protein, 448 aa, synthetic construct
  REGION 1..448: note = Synthetic polypeptide
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNYNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRWSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 213 aa, synthetic construct
  REGION 1..213: note = Synthetic polypeptide
DIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC